SEQUENCE LISTING (128 sequences)

SEQ ID NO: 1 - DNA, 1116 bp, Homo sapiens
  CDS (7)..(1107)
cgtgggatgttagcggtgggggcaatggagggcacccggcagagcgcattcctgctcagcagccctcccctggccgccctgcacagcatggccgagatgaagaccccgctgtaccctgccgcgtatcccccgctgcctgccggccccccctcctcctcgtcctcgtcgtcgtcctcctcgtcgccctccccgcctctgggcacccacaacccaggcggcctgaagcccccggccacgggggggctctcatccctcggcagccccccgcagcagctctcggccgccaccccacacggcatcaacgatatcctgagccggccctccatgcccgtggcctcgggggccgccctgccctccgcctcgccctccggttcctcctcctcctcttcctcgtccgcctctgcctcctccgcctctgccgccgccgcggctgctgccgcggccgcagccgccgcctcatccccggcggggctgctggccggactgccacgctttagcagcctgagcccgccgccgccgccgcccgggctctacttcagccccagcgccgcggccgtggccgccgtgggccggtaccccaagccgctggctgagctgcctggccggacgcccatcttctggcccggagtgatgcagagcccgccctggagggacgcacgcctggcctgtacccctcatcaaggatccattttgttggacaaagacgggaagagaaaacacacgagacccactttttccggacagcagatcttcgccctggagaagactttcgaacaaacaaaatacttggcggggcccgagagggctcgtttggcctattcgttggggatgacagagagtcaggtcaaggtctggttccagaaccgccggaccaagtggaggaagaagcacgctgccgagatggccacggccaagaagaagcaggactcggagacagagcgcctcaagggggcctcggagaacgaggaagaggacgacgactacaataagcctctggatcccaactcggacgacgagaaaatcacgcagctgttgaagaagcacaagtccagcagcggcggcggcggcggcctcctactgcacgcgtccgagccggagagctcatcctgaacgccg

SEQ ID NO: 2 - protein, 367 aa, Homo sapiens
MLAVGAMEGTRQSAFLLSSPPLAALHSMAEMKTPLYPAAYPPLPAGPPSSSSSSSSSSSPSPPLGTHNPGGLKPPATGGLSSLGSPPQQLSAATPHGINDILSRPSMPVASGAALPSASPSGSSSSSSSSASASSASAAAAAAAAAAAAASSPAGLLAGLPRFSSLSPPPPPPGLYFSPSAAAVAAVGRYPKPLAELPGRTPIFWPGVMQSPPWRDARLACTPHQGSILLDKDGKRKHTRPTFSGQQIFALEKTFEQTKYLAGPERARLAYSLGMTESQVKVWFQNRRTKWRKKHAAEMATAKKKQDSETERLKGASENEEEDDDYNKPLDPNSDDEKITQLLKKHKSSSGGGGGLLLHASEPESSS

SEQ ID NO: 3 - DNA, 19 bp, Rattus norvegicus
gagcacgcttggcctattc

SEQ ID NO: 4 - DNA, 19 bp, Rattus norvegicus
ggtattcacaccacttggc

SEQ ID NO: 5 - DNA, 19 bp, Rattus norvegicus
gtacttggcaggaccagag

SEQ ID NO: 6 - DNA, 19 bp, Rattus norvegicus
agcacaaatccagcggagg

SEQ ID NO: 7 - RNA, 47 nt, Artificial
  siRNA hairpin sequence
gagcacgcuuggccuauucuucaagagagaauaggccaagcgugcuc

SEQ ID NO: 8 - DNA, 21 bp, Artificial
  siRNA oligonucleotide sequence
gagcacgcuuggccuauuctt

SEQ ID NO: 9 - DNA, 21 bp, Artificial
  siRNA oligonucleotide sequence
gaauaggccaagcgugcuctt

SEQ ID NO: 10 - DNA, 47 bp, Artificial
  siRNA hairpin sequence
ggtattcacaccacttggcttcaagagagccaagtggtgtgaatacc

SEQ ID NO: 11 - DNA, 21 bp, Artificial
  siRNA oligonucleotide sequence
gguauucacaccacuuggctt

SEQ ID NO: 12 - DNA, 21 bp, Artificial
  siRNA oligonucleotide sequence
gguauucacaccacuuggctt

SEQ ID NO: 13 - DNA, 46 bp, Artificial
  siRNA hairpin sequence
agcacaaatccagcggaggttcaagagacctccgctggatttgtgc

SEQ ID NO: 14 - DNA, 21 bp, Artificial
  siRNA oligonucleotide sequence
agcacaaauccagcggaggtt

SEQ ID NO: 15 - DNA, 21 bp, Artificial
  siRNA oligonucleotide sequence
ccuccgcuggauuugugcutt

SEQ ID NO: 16 - DNA, 47 bp, Artificial
  siRNA hairpin sequence
gagcacgcttggcctattcttcaagagagaataggccaagcgtgctc

SEQ ID NO: 17 - DNA, 47 bp, Artificial
  siRNA hairpin sequence
gtacttggcaggaccagagttcaagagactctggtcctgccaagtac

SEQ ID NO: 18 - DNA, 21 bp, Artificial
  siRNA oligoncucleotide sequence
guacuuggcaggaccagagtt

SEQ ID NO: 19 - DNA, 21 bp, Artificial
  siRNA oligonucleotide sequence
cucugguccugccaaguactt

SEQ ID NO: 20 - DNA, 1989 bp, Homo sapiens
catcatctgacagataaatattcctattaaacggattcagttttcagcgaattgagtaacccataaatgataatgaacgcggtgggaagcgacgggcgggggggaactcgggaatgaaaaaaaaaataaagtggaggagaaagaacagaaaaggaaagcaggaggtggaaagatggaagaggacgatcctttggcctacaaggggattaaggacatctataaggcttaaggagcaacaaattaatttacacaattctgggagagcccagatggcctttaattaatcccttcaaaagaaggagccaggccagggctgcgccggctgcctgctccattagctccattttacaagggaccagacttggttcgaggtgaggcgccctccagagctggtgggggaaggggataggatgacgcgagcgggctagtggggaagcaagggaagaatatgaactgcttttccataaaagggctgagttttcattattcctctctttaaaaagtaataccctcttcgtctctgcttccccctcccctttctcattttatttagcacaattaattgaggcggccactggccccagcgcggaaccgcaccactcaccagctcccgcccctcctggccccgcccacaggagaaagaagtagggagcgggaggggactaggcgggcgcggccctacgcctggcccgcctcagccaatcagagggtgcggcgcccccgagtgggcgagccccaggggcgacgcaaggatcgaggcggcgagctattggacacggtggttacgcccccggcctgcgcccggctcgccggcccccgcagcctcggagtgacgtccctcaaagttctcattttggtcccccacttccccctccctttcgtcccccagctaaagaggggtagggagtgatgcaaatgttttattacctttgagagcttcatctgaactgtcaggcccggagggagagaggaaaggagagaaagagcggaggagccgcggagagggtcagtttggccagaggacaggacttagaagaccgaagcctgggaagccgcgaagaaaatgccagagaggagagtcaaaggctgagagtgagagggagagagggagcgggggtggggcgggggtcgccgggcccgtttgcagaagtggactgacgagcggcgccgaaacccagccgtcagacttttcacacttagtcttttgtttttctgtgtttttcctccccctttttcttttcaatattgcaactccagtgccccgtgggccagagggcaaggcgggtggaggtgaggacctgggagccgcggggatccgtggcactgccctttctggcgcagcagcccggggcagcgtgggcggaggaagcccgcacagaggctagatctcccgcgggctggatgcgctttctccccgggcacagtgagcgtcgaatgcgaatcagccgcgcgaccgaaagagcagagcatcccagtaagatcagaggagcgccacgggctgcacaaggcgtcctttgaacctccccaaagaaagcaagccacccccaccctccaacttcaaagtggagattcggcaactaactttgctacaaactctccggagccagcctgggttttgttttgcttatttcccgggggcagaagatgagaagtagcgcactttgaacagctaggaaaagtgaggaagagagaatagccagggatcgaatctaggactcgcggaacgaaaggactgcctagcccgccgggacgcctgcttttctcggcgagctgccgcctcccgcgtggagggtttggacatctctgctgcgcagctaggcgagcaactcccggcagcggcatttttggttcagttggcagctcgcctccgggcgcgccgagtgcctctccgctcgcgccctcggcgcttccggctcctctgagccccgcggggggcaccagccagcgccctcgctgcaaggctacggtctccggcgtggccgtgggatg

SEQ ID NO: 21 - DNA, 2460 bp, Mus musculus
ctgagcgaatgaaaggagcaggcgatgattaacccctaggctttactctcacacgtcaaactcaacctcaagttgacaacactcaagctttggagaagtaaggggaccgaccagtttaaggcctcctgcttgacattcagagtcaaactccctggcgctgctcagttttaattcctgggtcatttatcgcctatttctgttttctgaaccttaaatttggactcaataatatgatgcaaatctctgctgtgacacttcccgccccccccccccccactggtgtatcaactgcccgatttctcaagatcgaccaaagaggttttttccttggttttggtcaaccctgagcagaccttaaagatcggccagagggagcaaagcccttttgaccatcgctcccaatgtcagcctagaagtcggtcgtctctagtttactcaactacccgagttgagagcttgaccagactttccaacagttacctgtcttcccccgaggtattcctctatctaaagttgtcctgtgaattttagtgatcctgcctcataaatccaactaataataatagagggaggattttaaaaaaataattatctcatttctgttaggtttagacaccacgcaggagataaatattctcattaagctgatttcatccccagagtactgagccccctcataagtgataatgatctagggagtgggagagcgaagacaagaacggagaaagaacagaaaagagcaggagacggaaagatggtgaagggtgacccttaggcctgcgaggggtttaaaaacatctacgggcttaaggaacaacaaatcaatttacacgattctggaagagcccagagggcctttaattaatcccttcaaaagaaggaagtcggcctgggatatgcctcctgcctgctccattagctcccttttcgcaagggtccagacaccgttggaggtgggcgccgcgcgcgagctgggggaggatgacgcgagctggcgtgggcggaagagccgcacttaaactgcttttccatagaagggctggattttcattattcctctctttaaaaagtaatgccctcttcgtccctgctccctccttctcctttccattttattttgcacgattagttgagccggccgctggctctagactggaaccactcttttcgccaggcccctcccctcttggctccgcccaagtgaagctggggcggggactaggagggcgcgtccttatggctccctagtctcagccaatcaaaaggtgtggcgctcccaggtgggcgtgttctaggagcgacgccttgcccaagctgagcgctattggaggcggtgtttacgcccaggacccgggccccgctcctcagtcccgccccgccgagccgccccggaatgacgtcctcgaaagttctcattttggccccccacctcccctcccttgcgtcccccagctaaagagaggcagggaggggtgcaaatattttattacctttgagagcttcatccgaactgtcaggcccagagggagagaggagagaagggaagagccgccgagagggtcagtttggccagaggacagggcttggaagagccaagcctggaaagccaaggagaaatgccagagaggcaagagaaaggcggagagtgagaggaagagagggcgccagggggtgggacgggggggggtctcccggccggcttgcagaagaggacggacgatcggaaccggcccgtcagactttttccatacttaggtttattttcttttatccttttcgatccggctagtccggcgccgcgtggctcggaggggaagtaggctcgtgggcgcaccaagtccccagccgccggagcggtgtcactgactgccctttctggcgcagtaacgccgggcccgaggcagtggcgggcgcgatacccgcgccggccgtttgtgctctgtcccccgggcgagcggtgagtgcgactcagcagcgcggccgccgggagagcggagcgtccgagcgagatcagaggcgcgcaccgggcggaacgccgcccgctttgaagctcccccaggcgagcgagccggcccccgccctcctacatcaaagcgaacgctccgcgcctcccaaccttgttgcaaactctctgggtcggctgcggggtacgtcttgctgatttcccgcgggggtggagaagatgagaagcagagcgctctgagccgggaacgagggaccagcgcctgggatcgaatccgggactcccgaagccgaggaagcgctgagcccgcccgcgcccccgcagccctcgcccctgccgcctcccgcggggcgtttggacatttttgctgcgcagctcccggagcccgcggccgatccacacttcgcttgcgcgcgcccccggcacctcgggttctcccgagccccggcggggccaccgacctgcgtggctgcgggttcgggtctggctgtgggatg

SEQ ID NO: 22 - DNA, 2780 bp, Rattus norvegicus
gcctatgaagcggctccatgaaattggccgcctgctcttctctggtttaagctgcagaagcctttcctggcttggaagtgcagacagaacccactccagctctctcccgagcctagaggctggcaggccccttagcgtatctcccattcattcagaaccccacctctcctagggtcagatcctaaattctaccgggttgatagcattaaccaagtcttgaggctaataaaggtgggagagctggaggaagacagatatcatcttcccccacccccgacccgactccgagtgaaagaaaggggctaacgatgattaacccctagactttccgctcacacctctaatccaacctcaggttgacacccgtgaaagacccaagctttggcggaggtaagaggacaggatcaaggcctcctactggaagcaccttgacattcagagtcaaactccccggggcgctgctcggtttttattcttgggtcatttattgcatctatttctgtttcctgaaccttaaatttggactcaatgatatgatgtaaatctctactgtgacacttcccgcccccacactggtgtatcaaccgcctgatttctcaggatcgaccaaagaggtttcttttttctcctcagtttaggtcaaccctgaccagaccttaaagatcaagcagagggagcaaagccattttgaccatcgctcccaatcctagcccagaagtcagtagtctcttgctcactcaaatccccagagttgtgggcttgaccggactttccagcagcttcctgtctcccccgaggtattcctctgtctaaagttgtcgcgtgaattttagtgatcctgcctcgtgtgatccaactaataataataggaggaattttaaaaaatcatctcatttttcttagggttagacaccacgcaggagatacatagtctcgttaagttgatttcatccccggagtagtgagccccctcgtgagtgataatgatctagggggagggagagtgaaaacaagaacggaggaagaatagaaaagagcaggagatggaaagatgggcgaggatggcccttaggcctgcgaggggatttaaaaacatctatgggcttaaggagcaacaaatcaatttacacaattctggaagagcccagatggcctttaattaatcccttcaaaagaaggaaccctgcctgggacgtgcctcctgcctgctccattagctccctttccgcaagggtccagacaccagtcgaggtaaggtgctgcgagctagtgggggaaatgtgggaggacggaagagatgaacttaaactgcttttccataaaagggcggtcttttcattattcctctctttaaaaagtaatgccctcttcgtccctgctccctccttctcttttccattttattttgcacaattaattgagccggccgctgtccctaggctggaaccactcttttggccaggcccctcccctcttggctccgcccaaagaaagctggggcggggattaggcgggcgcgcccttgcggctccctagtctcagccaatcaaaaggcatggcgctccccggtgggcgtgtcccaggagcgacgcctcgctgaggctgagcgctattggatgcggtggctacgcccaggctctgcactccgaccctcagtcccgcctctcggagccgcccctgagtgacgtcctcgaaagttctcattttggccccccgcctcccctccctcgcgtcccccagctaaagagcgacagggaggggtgcaaatgttttattacctttgagagcttcatccgaactgtcaggcccggagggagagaggagagaagggaagagccgccgagagggtcagtttggccagaggacagggctcggaagagccaagcctggaaagccaaggagaaaggccagagaggcgagagaaaggcggagagtgagaggaagcgagggcgccgggggggtggggcgggggactcggggccggcttgcacaagcggacggacgatcggaaccggcccgtcagacttttcccacttaggtttattttctctttcttcctttttcggtcccgctactccggcgccgcgtggcccggaggggaagcaggcgcgtgggcgcgccaagtgcccagccgccggagcggtgccgccactgccctttctggcgcagtaacgccggtcccacggcggtagcgggcgcgatctccgcgagagccgtttgcgctccatcccctgggcgagcggcgagtgcgactcagctgcgcggccgccgggagcgcggagcgtccgagcgagaacagaggcgcgcaccgggcggaacgcggcccgctttgaagctcccctaggcgggcgagccggcccccaccctcttacatcaaagcgatcgctccgcgcctcccaaccttgttgcaaactctctgggccagccccgggtacgtcttcctgatttcccgcgggggtggagagatgagaagcagagctctctgagtcgggagcgagggaccagcgcccgggatcgaatcccggactcccggagtcgagaaagcgccaagcgggcccgcgcccctgccgccctcgcccgccgccgcctcccgcggggcatttggacattttctgctgcgcagctcgcaccccagctctcggagcccgccgcagagtcacacttcgcttgcgcgcgagcgcttccccagcgcctccggctctcctgcgcctcgggctctcctgagccccggcggggccaccggcctgcttggctgcgggttcgggtctggctgtgggatg

SEQ ID NO: 23 - DNA, 19 bp, Rattus norvegicus
gaaagaggaagataagaaa

SEQ ID NO: 24 - DNA, 19 bp, Artificial
  Random siRNA sequence
gagaccctatccgtgatta

SEQ ID NO: 25 - DNA, 30 bp, Artificial
  EMSA oligonucleotide
gaacaaaaccccattatttacagatgagaa

SEQ ID NO: 26 - DNA, 23 bp, Artificial
  PCR primer
ggatccttcagagagctgaatag

SEQ ID NO: 27 - DNA, 24 bp, Artificial
  PCR primer
ggcaagcttcaccagggtgctgtg

SEQ ID NO: 28 - DNA, 21 bp, Artificial
  PCR primer
ccacttcgtccttggctcaac

SEQ ID NO: 29 - DNA, 22 bp, Artificial
  PCR primer
gggataccaggcacagcatagg

SEQ ID NO: 30 - DNA, 23 bp, Artificial
  PCR primer
gctctgccatttatcatcactgg

SEQ ID NO: 31 - DNA, 20 bp, Artificial
  PCR primer
tgactgccaagcaaggaagc

SEQ ID NO: 32 - DNA, 24 bp, Artificial
  PCR primer
caggctttctgtagcagtgaggtg

SEQ ID NO: 33 - DNA, 23 bp, Artificial
  PCR primer
ggtttctggtgtgtgtagcgaag

SEQ ID NO: 34 - DNA, 22 bp, Artificial
  PCR primer
tctcctgcccctaccgttttac

SEQ ID NO: 35 - DNA, 24 bp, Artificial
  PCR primer
aacagatagcacccagaccctctc

SEQ ID NO: 36 - DNA, 21 bp, Artificial
  PCR primer
ccacttcgtccttggctcaac

SEQ ID NO: 37 - DNA, 22 bp, Artificial
  PCR primer
gggataccaggcacagcatagg

SEQ ID NO: 38 - DNA, 27 bp, Artificial
  PCR primer
caactcgaggctgagctaagaatccag

SEQ ID NO: 39 - DNA, 27 bp, Artificial
  PCR primer
gcagaattcctgcttgctgatggtcta

SEQ ID NO: 40 - DNA, 21 bp, Artificial
  PCR primer
gagttcatgcgcttcaaggtg

SEQ ID NO: 41 - DNA, 21 bp, Artificial
  PCR primer
ttggagtccacgtagtagtag

SEQ ID NO: 42 - DNA, 21 bp, Artificial
  PCR primer
gatggtgatgtcctcgttgtg

SEQ ID NO: 43 - DNA, 21 bp, Artificial
  PCR primer
gcgtgagtgtatctccatcca

SEQ ID NO: 44 - DNA, 21 bp, Artificial
  PCR primer
ggtaggtgccagtgcgaactt

SEQ ID NO: 45 - DNA, 22 bp, Artificial
  PCR primer
gacctgcagagctccaatcaac

SEQ ID NO: 46 - DNA, 22 bp, Artificial
  PCR primer
cacgaccctcagtaccaaaggg

SEQ ID NO: 47 - DNA, 21 bp, Artificial
  PCR primer
caaactgaacaagccactgag

SEQ ID NO: 48 - DNA, 20 bp, Artificial
  PCR primer
tctgctgtcgtcagactcac

SEQ ID NO: 49 - DNA, 23 bp, Artificial
  PCR primer
gacattttgtgaataaaccgaag

SEQ ID NO: 50 - DNA, 19 bp, Artificial
  PCR primer
gtggatggcagtgagggag

SEQ ID NO: 51 - DNA, 20 bp, Artificial
  PCR primer
atcattcccagcttcccaga

SEQ ID NO: 52 - DNA, 20 bp, Artificial
  PCR primer
cggttcctcttggtgttcat

SEQ ID NO: 53 - DNA, 21 bp, Artificial
  PCR primer
tcctgcccctgctggccctgc

SEQ ID NO: 54 - DNA, 20 bp, Artificial
  PCR primer
agttgcagtaggttctccag

SEQ ID NO: 55 - DNA, 24 bp, Artificial
  PCR primer
ggggagcgtggattcttctacaca

SEQ ID NO: 56 - DNA, 21 bp, Artificial
  PCR primer
tgcccacagaaatgtgccccc

SEQ ID NO: 57 - DNA, 24 bp, Artificial
  PCR primer
gcacctttgtggttctcacttggt

SEQ ID NO: 58 - DNA, 24 bp, Artificial
  PCR primer
cgggacatgggtgtgtagaagaat

SEQ ID NO: 59 - DNA, 20 bp, Artificial
  PCR primer
tctctgctgcctgcggacct

SEQ ID NO: 60 - DNA, 23 bp, Artificial
  PCR primer
gccaagaagtacttggccagttc

SEQ ID NO: 61 - DNA, 20 bp, Artificial
  PCR primer
cctcttccccatctggttcc

SEQ ID NO: 62 - DNA, 21 bp, Artificial
  PCR primer
tcacgtagtatacacctgctg

SEQ ID NO: 63 - DNA, 21 bp, Artificial
  PCR primer
cagatcctggcagagttccag

SEQ ID NO: 64 - DNA, 21 bp, Artificial
  PCR primer
acttctgagccttctggggtg

SEQ ID NO: 65 - DNA, 22 bp, Artificial
  PCR primer
gacaccccactcatagtcacac

SEQ ID NO: 66 - DNA, 21 bp, Artificial
  PCR primer
cagcaatgatgagagcatgtg

SEQ ID NO: 67 - DNA, 19 bp, Artificial
  PCR primer
tgtacgccatgcggctctc

SEQ ID NO: 68 - DNA, 19 bp, Artificial
  PCR primer
ctcgtggttcacctgcacc

SEQ ID NO: 69 - DNA, 22 bp, Artificial
  PCR primer
ggaacaaacttccgggtcctgc

SEQ ID NO: 70 - DNA, 19 bp, Artificial
  PCR primer
gtctaggctgttctgctgg

SEQ ID NO: 71 - DNA, 19 bp, Artificial
  PCR primer
tggaggtgctaaagcaggc

SEQ ID NO: 72 - DNA, 19 bp, Artificial
  PCR primer
atgcctccttggctgatgg

SEQ ID NO: 73 - DNA, 20 bp, Artificial
  PCR primer
tcacgcccaccaagcaggtc

SEQ ID NO: 74 - DNA, 19 bp, Artificial
  PCR primer
gttggatggcagcaggtgg

SEQ ID NO: 75 - DNA, 20 bp, Artificial
  PCR primer
gctcaccgtgcactttaacc

SEQ ID NO: 76 - DNA, 18 bp, Artificial
  PCR primer
gcaggcaaaccggtggtc

SEQ ID NO: 77 - DNA, 22 bp, Artificial
  PCR primer
cttggccaagaactacatctgg

SEQ ID NO: 78 - DNA, 19 bp, Artificial
  PCR primer
ctggtaggagtagggatgc

SEQ ID NO: 79 - DNA, 18 bp, Artificial
  PCR primer
gcctggggtgatgcagag

SEQ ID NO: 80 - DNA, 20 bp, Artificial
  PCR primer
cttctttctccacttggtcc

SEQ ID NO: 81 - DNA, 19 bp, Artificial
  PCR primer
caccagacccacattctcc

SEQ ID NO: 82 - DNA, 23 bp, Artificial
  PCR primer
ctcggctgcgtgcgtgcttcttt

SEQ ID NO: 83 - DNA, 19 bp, Artificial
  PCR primer
cacgagacccacgttctct

SEQ ID NO: 84 - DNA, 18 bp, Artificial
  PCR primer
tcagccgcgtgcttcttc

SEQ ID NO: 85 - DNA, 21 bp, Artificial
  PCR primer
aactatgtttgtgtgagtagc

SEQ ID NO: 86 - DNA, 20 bp, Artificial
  PCR primer
cagaacgtcatttggcaacg

SEQ ID NO: 87 - DNA, 18 bp, Artificial
  PCR primer
ccgtgcgcattccagagg

SEQ ID NO: 88 - DNA, 20 bp, Artificial
  PCR primer
cttcgtatggggagatgtcc

SEQ ID NO: 89 - DNA, 19 bp, Artificial
  PCR primer
ccagtgtgaagcatgcagc

SEQ ID NO: 90 - DNA, 19 bp, Artificial
  PCR primer
gtggccagacgtggtttgc

SEQ ID NO: 91 - DNA, 17 bp, Artificial
  PCR primer
gcctggtcagcggaagc

SEQ ID NO: 92 - DNA, 19 bp, Artificial
  PCR primer
tctcctcccgcttgatctc

SEQ ID NO: 93 - DNA, 20 bp, Artificial
  PCR primer
cttctgatgagttggaacag

SEQ ID NO: 94 - DNA, 20 bp, Artificial
  PCR primer
gatcttgtcaatgctggtcg

SEQ ID NO: 95 - DNA, 24 bp, Artificial
  PCR primer
tcgttgagaagaactcagccagca

SEQ ID NO: 96 - DNA, 24 bp, Artificial
  PCR primer
agcgtaaccagacgtctcaacact

SEQ ID NO: 97 - DNA, 24 bp, Artificial
  PCRR primer
tcaaaggcggctacaaggagttct

SEQ ID NO: 98 - DNA, 24 bp, Artificial
  PCR primer
agaactccttgtagccgcctttga

SEQ ID NO: 99 - DNA, 24 bp, Artificial
  PCR primer
agcttgcccgagttctactacaga

SEQ ID NO: 100 - DNA, 24 bp, Artificial
  PCR primer
tttgcctgagacccaattgaaggc

SEQ ID NO: 101 - DNA, 24 bp, Artificial
  PCR primer
ttagagaacttccagggcaagcga

SEQ ID NO: 102 - DNA, 24 bp, Artificial
  PCR primer
ttccacttggatagagcccacctt

SEQ ID NO: 103 - DNA, 24 bp, Artificial
  PCR primer
tcgagtccaagtgttccatgggtt

SEQ ID NO: 104 - DNA, 24 bp, Artificial
  PCR primer
acggagacaggtcaatcacaccaa

SEQ ID NO: 105 - DNA, 24 bp, Artificial
  PCR primer
agagttacgccaagggaagcagaa

SEQ ID NO: 106 - DNA, 24 bp, Artificial
  PCR primer
aaatcggtgaggcagagaagcaga

SEQ ID NO: 107 - DNA, 24 bp, Artificial
  PCR primer
tacttggctggtggctctttgact

SEQ ID NO: 108 - DNA, 24 bp, Artificial
  PCR primer
atccaaagcttggaggcactctct

SEQ ID NO: 109 - DNA, 24 bp, Artificial
  PCR primer
ttgtggaggaggcagagaatggaa

SEQ ID NO: 110 - DNA, 24 bp, Artificial
  PCR primer
taggagcctcagcttcctcatctt

SEQ ID NO: 111 - DNA, 24 bp, Artificial
  PCR primer
agaatggtctgggagcaaaggtca

SEQ ID NO: 112 - DNA, 24 bp, Artificial
  PCR primer
acacacccggatgttcccataagt

SEQ ID NO: 113 - DNA, 24 bp, Artificial
  PCR primer
aaccacgcgagctgtcaggataat

SEQ ID NO: 114 - DNA, 24 bp, Artificial
  PCR primer
tcaaggcagagcatcttcaggtca

SEQ ID NO: 115 - DNA, 24 bp, Artificial
  PCR primer
ttaacgagctgaacctgcccaaga

SEQ ID NO: 116 - DNA, 24 bp, Artificial
  PCR primer
accctgtcccaccttaaagctgaa

SEQ ID NO: 117 - DNA, 24 bp, Artificial
  PCR primer
actgggtcgtgaagtcactggaaa

SEQ ID NO: 118 - DNA, 24 bp, Artificial
  PCR primer
aacatggcagttacaccaaccagc

SEQ ID NO: 119 - DNA, 24 bp, Artificial
  PCR primer
tgtgtcaggctttctccgatgtga

SEQ ID NO: 120 - DNA, 24 bp, Artificial
  PCR primer
tttccagtgacttcacgacccagt

SEQ ID NO: 121 - DNA, 24 bp, Artificial
  PCR primer
gtcagtgaacaactgcaggccaaa

SEQ ID NO: 122 - DNA, 24 bp, Artificial
  PCR primer
ttaacaggctcaggttctggctca

SEQ ID NO: 123 - DNA, 17 bp, Rattus norvegicus
ttggcatttttgccttg

SEQ ID NO: 124 - DNA, 17 bp, Rattus norvegicus
tttttattttctaagta

SEQ ID NO: 125 - DNA, 17 bp, Rattus norvegicus
gaaggattttccccagg

SEQ ID NO: 126 - DNA, 17 bp, Artificial
  PCR primer
ggaggattttgtgtggt

SEQ ID NO: 127 - DNA, 17 bp, Artificial
  PCR primer
gttgtatttttcttaag

SEQ ID NO: 128 - DNA, 17 bp, Rattus norvegicus
ttctcattttggccccc

(19) United States
(12) Patent Application Publication
(10) Pub. No.: US 2014/0315984 A1
(43) Pub. Date: Oct. 23, 2014

(54) Methods and Compositions for Treatment of Diabetes and Related Disorders

(71) Applicant: Duke University, Durham, NC (US)

(72) Inventors: Christopher B. Newgard, Chapel Hill, NC (US); Jonathan C. Schisler, Morrisville, NC (US)

(73) Assignee: Duke University, Durham, NC (US)

(21) Appl. No.: 14/164,865

(22) Filed: Jan. 27, 2014

Related U.S. Application Data
(62) Division of application No. 11/709,577, filed on Feb. 22, 2007, now abandoned.
(60) Provisional application No. 60/776,034, filed on Feb. 23, 2006.

(51) Int. Cl. C07K 14/47

(52) U.S. Cl. CPC C07K 14/47
USPC 514/44 R; 435/455; 435/456

(57) ABSTRACT

The present invention provides a method of stimulating growth of a pancreatic islet beta cell and/or enhancing glucose stimulated insulin secretion of a pancreatic islet beta cell, comprising delivering to the cell an exogenous nucleotide sequence encoding Nkx6.1 transcription factor.

### PRIORITY STATEMENT

This application claims the benefit, under 35 U.S.C. §119(e), of U.S. Provisional Application No. 60/776,034, filed Feb. 23, 2006, the entire contents of which are incorporated by reference herein.

### GOVERNMENT SUPPORT

Aspects of this invention were supported by government funds provided by the National Institutes of Health Grant Nos. U01-DK-56047 and RO1-DK-58398. The U.S. Government has certain rights in this invention.

### FIELD OF THE INVENTION

The present invention relates to compositions and methods of their use in stimulating growth of pancreatic islet beta cells and enhancing glucose stimulated insulin secretion, having application, for example in the treatment of diabetes.

### BACKGROUND OF THE INVENTION

The islets of Langerhans are comprised of at least four cell types that produce specific hormones, including insulin-producing beta cells and glucagon-producing alpha cells that play a critical role in maintenance of metabolic fuel homeostasis. Type 1 diabetes (insulin-dependent diabetes mellitus (IDDM)) results from autoimmune destruction of the beta cells, whereas type 2 diabetes (non-insulin dependent diabetes mellitus (NDDM)), involves loss of glucose-stimulated insulin secretion (GSIS) and a gradual diminution of beta cell mass. Insulin injection therapy has been the standard treatment for type 1 diabetes since the discovery of the hormone more than 80 years ago. Islet transplantation has been intensively investigated as an alternative approach to insulin replacement in patients with type 1 diabetes but a major obstacle to broad application of this therapeutic approach continues to be an inadequate supply of fully differentiated human islets. Pharmacotherapy of type 2 diabetes includes administration of agents that enhance insulin secretion, but these drugs often lose efficacy over time and result in complications such as hypoglycemia. Thus, there is a need in the art for improved therapeutic agents for the treatment of diabetes.

The present invention overcomes previous shortcomings in the art by providing compositions and methods for their use in treating disorders associated with pancreatic islet beta cells and insulin secretion.

### SUMMARY OF THE INVENTION

The present invention provides a method of stimulating growth of a cell of this invention (e.g., a pancreatic islet beta cell), comprising delivering to the cell an exogenous nucleotide sequence encoding Nkx6.1 or an active fragment thereof.

Also provided herein is a composition comprising a cell or a population of cells comprising an exogenous nucleotide sequence encoding Nkx6.1 or an active fragment thereof and a pharmaceutically acceptable carrier.

Additionally provided herein is a method of treating diabetes in a subject, comprising delivering to the subject a cell of this invention (e.g. a pancreatic islet beta cell) comprising an exogenous nucleotide sequence encoding Nkx6.1 or an active fragment thereof.

Furthermore, the present invention provides a method of treating diabetes in a subject, comprising delivering to the subject an exogenous nucleotide sequence encoding Nkx6.1 or an active fragment thereof.

In additional embodiments, the present invention provides a method of increasing pancreatic islet beta cell mass and/or of increasing glucose-stimulated insulin secretion from pancreatic islet beta cells in a subject, comprising delivering an exogenous nucleotide sequence encoding Nkx6.1 transcription factor to pancreatic islet beta cells of the subject and/or delivering to the subject a cell of this invention comprising an exogenous nucleotide sequence encoding Nkx6.1 or an active fragment thereof.

Also provided herein is a method of suppressing growth of a cell of this invention, comprising delivering to the cell a nucleic acid comprising a nucleotide sequence that suppresses expression of a Nkx6.1 gene and/or of a Nkx6.1 activity.

In yet further embodiments, a method is provided of treating a disorder associated with hyperproliferation of pancreatic islet beta cells or hypersecretion of insulin in a subject, comprising delivering to the subject a nucleic acid comprising a nucleotide sequence that suppresses expression of a Nkx6.1 gene.

The present invention additionally provides a method of decreasing pancreatic islet beta cell mass in a subject, comprising delivering to the subject a nucleic acid comprising a nucleotide sequence that suppresses expression of a Nkx6.1 gene.

Further provided is a method of decreasing glucose stimulated insulin secretion from pancreatic islet beta cells in a subject, comprising delivering to the subject a nucleic acid comprising a nucleotide sequence that suppresses expression of a Nkx6.1 gene.

In additional embodiments of this invention, provided herein is a method of identifying a substance having the ability to enhance Nkx6.1 activity in a cell or a population of cells, wherein the cell or the cells of the population comprise an exogenous nucleotide sequence encoding Nkx6.1, comprising:

a) measuring the amount of cell proliferation in the cell or the population of cells in the absence of the substance;

b) measuring the amount of cell proliferation in the cell or the population of cells in the presence of the substance; and

c) comparing the amount of cell proliferation of (a) with the amount of cell proliferation of (b), whereby an increase in the amount of cell proliferation of (b) identifies a substance having the ability to enhance Nkx6.1 activity in a cell or a population of cells.

Further provided herein is a method of identifying a substance having the ability to suppress Nkx6.1 activity in a cell or a population of cells, wherein the cell or cells of the population comprise an exogenous nucleotide sequence encoding Nkx6.1, comprising:

a) measuring the amount of cell proliferation in the cell or the population of cells in the absence of the substance;

b) measuring the amount of cell proliferation in the population of cells in the presence of the substance; and

c) comparing the amount of cell proliferation of (a) with the amount of cell proliferation of (b), whereby a decrease in the amount of cell proliferation of (b) identifies a substance having the ability to suppress Nkx6.1 activity in a cell or a population of cells.

In addition, the present invention provides a method of identifying a substance having the ability to enhance Nkx6.1 gene expression and/or Nkx6.1 activity in a cell comprising a nucleotide sequence encoding a promoter region of a Nkx6.1 gene operably linked to a nucleotide sequence encoding a reporter protein, comprising:

a) measuring the amount of the reporter protein produced and/or the amount of report protein activity in the cell in the absence of the substance;

b) measuring the amount of the reporter protein produced and/or the amount of reporter protein activity in the cell in the presence of the substance; and

c) comparing the amount of reporter protein produced and/or reporter protein activity of (a) with the amount of reporter protein produced and/or reporter protein activity of (b), whereby an increase in the amount of reporter protein produced and/or reporter protein activity of (b) identifies a substance having the ability to enhance Nkx6.1 gene expression and/or Nkx6.1 activity in the cell.

Furthermore, the present invention provides a method of identifying a substance having the ability to suppress or reduce Nkx6.1 gene expression and/or Nkx6.1 activity in a cell comprising a nucleotide sequence encoding a promoter region of a Nkx6.1 gene operably linked to a nucleotide sequence encoding a reporter protein, comprising:

a) measuring the amount of the reporter protein produced and/or the amount of report protein activity in the cell in the absence of the substance;

b) measuring the amount of the reporter protein produced and/or the amount of reporter protein activity in the cell in the presence of the substance; and

c) comparing the amount of reporter protein produced and/or reporter protein activity of (a) with the amount of reporter protein produced and/or reporter protein activity of (b), whereby a decrease in the amount of reporter protein produced and/or reporter protein activity of (b) identifies a substance having the ability to reduce or suppress Nkx6.1 gene expression and/or Nkx6.1 activity in the cell.

These and other aspects of the present invention will be discussed in more detail in the description of the invention set forth below.

### DETAILED DESCRIPTION OF THE INVENTION

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

Except as otherwise indicated, standard methods can be used for the production of viral and non-viral vectors, manipulation of nucleic acid sequences, production of transformed cells, and the like according to the present invention. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

### I. DEFINITIONS

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The term “modulate,” “modulates” or “modulation” refers to enhancement (e.g., an increase) or inhibition (e.g., a reduction) in the specified activity.

The term “overexpress,” “overexpresses” or “overexpression” as used herein in connection with an isolated nucleic acid encoding a polypeptide refers to expression that results in higher levels of polypeptide production than exist in the cell in its native (control) state.

The term “overexpress,” “overexpresses” or “overexpression” as used herein in connection with isolated nucleic acids encoding a Nkx6.1 transgene refers to expression that results in higher levels of NKX6.1 polypeptide than exist in the cell in its native (control) state. Overexpression of NKX6.1 can result in levels that are 25%, 50%, 100%, 200%, 500%, 1000%, 2000% or higher in the cell. Further, nucleic acid encoding Nkx6.1 can be introduced into a cell that does not produce the specified form of Nkx6.1 (e.g., an isoform) encoded by the transgene or does so only at negligible levels.

As used herein, the term “diabetes” is used interchangeably with the term “diabetes mellitus.” The terms “diabetes” and “diabetes mellitus” are intended to encompass both insulin dependent and non-insulin dependent (Type 1 and Type 2, respectively) diabetes mellitus, unless one condition or the other is specifically indicated.

The term “insulin secretion” (IS) as used herein refers to secretion of insulin from a cell, e.g., into the systemic circulation or cell culture medium, and will typically refer to secretion of insulin from pancreatic islet beta-cells, although it can also refer to secretion from cells which have been engineered to express a recombinant insulin (for example, artificial beta-cells). Insulin secretion can be assessed directly, for example, by measuring media or plasma insulin concentrations using art-known methods such as radioimmunoassay. Alternatively, insulin secretion can be indirectly evaluated by measuring, for example, changes in plasma glucose concentrations (or glucose concentrations in cell culture medium).

“Glucose-stimulated insulin secretion” refers to the compensatory secretion of insulin in response to an elevation in serum glucose (e.g., following a meal or a glucose challenge) or, in the case of cultured cells, to an elevation of glucose concentration in the cell culture medium. In particular embodiments, glucose-stimulated insulin secretion results in an increase of at least about 2-fold, 3-fold, 4-fold, 5-fold, 6-fold, 8-fold, 10-fold, twelve-fold, or even fifteen-fold or more in insulin secretion over baseline levels in the presence of an increased concentration of glucose. In other words, the level of glucose-stimulated insulin secretion can be dependent on the glucose concentration, but the maximal elevation in insulin secretion is at least about 2-fold, 3-fold, 4-fold, 5-fold, 6-fold, 8-fold, 10-fold, twelve-fold, or even fifteen-fold or more over baseline levels. Alternatively, glucose-stimulated insulin secretion can be commenced in a cell or subject that did not previously have any detectable glucose-stimulated insulin secretion (or only negligible levels).

The term “enhance,” “enhances,” “enhancing” or “enhancement” with respect to insulin secretion refers to an increase in insulin secretion (e.g., at least about a 1.5-fold, 2-fold, 3-fold, 4-fold, 5-fold, 6-fold, 8-fold, 10-fold, twelve-fold, or even fifteen-fold or more increase), for example, in response to elevated glucose concentrations. Alternatively, these terms can refer to commencing insulin secretion in a cell or subject that did not previously have any detectable insulin secretion. In particular embodiments, fuel-stimulated IS is enhanced in a cell or subject comprising an isolated nucleic acid encoding NKX6.1 according to the invention as compared with the level of fuel-stimulated IS in a comparable cell in the absence of the isolated nucleic acid overexpressing NKX6.1.

By “providing fuel-simulated [or glucose-stimulated] insulin secreting capability” to a subject, it is meant that fuel-stimulated (or glucose-stimulated) insulin secreting capability is enhanced as described above. Thus, fuel-stimulated (or glucose-stimulated) insulin secretion can be commenced in a subject that did not previously have detectable fuel-stimulated (or glucose-stimulated) insulin secretion or can be increased above previous levels.

The term “glucose tolerance” refers to a state in which there is proper functioning of the homeostatic mechanisms by which insulin is secreted in response to an elevation in serum glucose concentrations. Impairment in this system results in transient hyperglycemia as the organism is unable to maintain normoglycemia following a glucose load (for example, a carbohydrate containing meal) because of insufficient secretion of insulin from the islet beta-cells or because of insensitivity of target tissues to circulating insulin.

“An improvement in glucose tolerance” is a level of amelioration in glucose tolerance that provides some clinical benefit to the subject. Glucose tolerance can be assessed by methods known in the art, such as for example, the oral glucose tolerance test, which monitors serum glucose concentrations following an oral glucose challenge. In particular embodiments, an “improvement in glucose tolerance” can result in normalization of fasting or baseline serum glucose concentrations, a reduction in maximal serum glucose concentrations, and/or an improved temporal response to a glucose challenge.

A “transgenic” non-human animal is a non-human animal that comprises a foreign nucleic acid incorporated into the genetic makeup of the animal, such as for example, by stable integration into the genome or by stable maintenance of an episome (e.g., derived from EBV).

A “therapeutically effective” or “effective” amount as used herein is an amount of a composition of this invention that provides some improvement or benefit to the subject. Alternatively stated, a “therapeutically-effective” or “effective” amount is an amount that provides some alleviation, mitigation, or decrease in at least one clinical symptom of glucose intolerance or diabetes in the subject (e.g., improved glucose tolerance, enhanced glucose-stimulated insulin secretion, and the like) or in at least one clinical symptom of a disorder associated with hypersecretion of insulin or hyperproliferation of pancreatic islet beta cells (e.g., more normalized insulin levels, etc.) as is well-known in the art. Those skilled in the art will appreciate that the therapeutic effects need not be complete or curative, as long as some benefit is provided to the subject.

By the terms “treat,” “treating” or “treatment of,” it is intended that the severity of the patient's condition is reduced or at least partially improved or modified and that some alleviation, mitigation or decrease in at least one clinical symptom is achieved.

As used herein, a “vector” or “delivery vector” can be a viral or non-viral vector that is used to deliver a nucleic acid to a cell, tissue or subject.

A “recombinant” vector or delivery vector refers to a viral or non-viral vector that comprises one or more heterologous nucleotide sequences (i.e., transgenes), e.g., two, three, four, five or more heterologous nucleotide sequences. Generally, the recombinant vectors and delivery vectors of the invention comprise a nucleotide sequence that encodes Nkx6.1, but can also comprise one or more additional heterologous sequences.

As used herein, the term “viral vector” or “viral delivery vector” can refer to a virus particle that functions as a nucleic acid delivery vehicle, and which comprises the vector genome packaged within a virion. Alternatively, these terms can be used to refer to the vector genome when used as a nucleic acid delivery vehicle in the absence of the virion.

A viral “vector genome” refers to the viral genomic DNA or RNA, in either its naturally occurring or modified form. A “recombinant vector genome” is a viral genome (e.g., vDNA) that comprises one or more heterologous nucleotide sequence(s).

A “heterologous nucleotide sequence” or “exogenous nucleotide sequence” will typically be a sequence that is not naturally-occurring in the vector. Alternatively, a heterologous nucleotide sequence or exogenous nucleotide sequence can refer to a sequence that is placed into a non-naturally occurring environment or is present in a cell or other environment in an amount that is greater than an endogenous amount (e.g., by association with a promoter with which it is not naturally associated; in a cell that does not contain an endogenous form of the heterologous nucleotide sequence or exogenous nucleotide sequence and/or under the direction of a promoter and/or other regulatory elements with which it is not normally associate; and/or in a cell that does contain an endogenous form of the heterologous nucleotide sequence or exogenous nucleotide sequence and presence of the heterologous nucleotide sequence or exogenous nucleotide sequence results in an increase that is greater than the endogenous amount of the nucleotide sequence). The terms “heterologous” and “exogenous” are interchangeable terms as used herein.

By “infectious,” as used herein, it is meant that a virus can enter or be introduced into a cell by natural transduction mechanisms and express viral genes and/or nucleic acids (including transgenes). Alternatively, an “infectious” virus is one that can enter the cell by other mechanisms and express the coding sequences carried within the viral genome therein. As one illustrative example, the vector can enter a target cell by having a ligand or binding protein on the virus particle that is specific for a cell-surface molecule of receptor and/or by using an antibody directed against a molecule on the cell surface of the virus particle, resulting in complex formation between the antibody and virus particle, followed by internalization of the complex.

As used herein, the term “polypeptide” encompasses both peptides and proteins, unless indicated otherwise.

A “fusion polypeptide” is a polypeptide produced when two heterologous nucleotide sequences or fragments thereof coding for two (or more) different polypeptides not found fused together in nature are fused together in the correct translational reading frame. Illustrative fusion polypeptides include fusions of NKX6.1 (or a portion thereof) to all or a portion of glutathione-S-transferase, maltose-binding protein, or a reporter protein (e.g., Green Fluorescent Protein, β-glucuronidase, β-galactosidase, luciferase, etc.).

As used herein, a “functional” polypeptide is one that retains at least one biological activity normally associated with that polypeptide. Preferably, a “functional” polypeptide retains all of the activities possessed by the unmodified peptide. By “retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits essentially no detectable biological activity normally associated with the polypeptide (e.g., at most, only an insignificant amount, e.g., less than about 10% or even 5%).

An “active fragment” of a Nkx6.1 protein of this invention is an amino acid sequence having fewer than all of the amino acids of the full or complete amino acid sequence of the Nkx6.1 and that retains one or more activity associated with the Nkx6.1 protein. Activities of the Nkx6.1 protein include, but are not limited to repressor activity (e.g. activity of the repressor domain at amino acids 91-268), DNA recognition (e.g., activity of the homeodomain at amino acids 229-305, activation (e.g., activity of the activation domain at amino acids 306-322, and binding interference (e.g., activity of the binding interference domain at amino acids 306-364.

A “biologically active fragment” or “active fragment” as used herein includes a polypeptide of this invention that comprises a sufficient number of amino acids to have one or more of the biological activities of the polypeptides of this invention. Such biological activities can include, but are not limited to, in any combination, binding activity, binding interference activity, repressor activity, activation activity, as well as any other activity now known or later identified for the polypeptides and/or fragments of this invention. A fragment of a polypeptide of this invention can be produced by methods well known and routine in the art. Fragments of this invention can be produced, for example, by enzymatic or other cleavage of naturally occurring peptides or polypeptides or by synthetic protocols that are well known. Such fragments can be tested for one or more of the biological activities of this invention according to the methods described herein, which are routine methods for testing activities of polypeptides, and/or according to any art-known and routine methods for identifying such activities. Such production and testing to identify biologically active fragments of the polypeptides described herein would be well within the scope of one of ordinary skill in the art and would be routine.

Fragments of the polypeptides of this invention are preferably at least about ten amino acids in length and retain one or more of the biological activities of the Nkx6.1 protein. Examples of the fragments of this invention include, but are not intended to be limited to, the following fragments identified by the amino acid number as shown in the Sequence Listing for SEQ ID NO:2: Amino acids 1-10, 10-20, 20-30, 30-40, 40-50, 50-60, 60-70, 70-80, 80-90, 90-100, 110-120, 120-130, 130-140, 140-150, 150-160, 160-170, 170-180, 180-190, 190-200, 200-210, 210-220, 220-230, 230-240, 240-250, 1-25, 1-50, 1-67, 1-75, 1-100, 1-125, 1-135, 1-145, 1-150, 1-160, 1-170, 1-180, 1-190, 1-200, 1-250, 68-180, 183-223, 50-100, 100-200, 200-300, 300-350, 300-364, etc.

It is understood that this list is exemplary only and that a fragment of this invention can be any amino acid sequence containing any combination of contiguous amino acids that are numbered in the Sequence Listing as amino acids 1 through 364 even if that combination is not specifically recited as an example herein. It is also understood that these fragments can be combined in any order or amount. For example, fragment 1-10 can be combined with fragment 10-20 to produce a fragment of amino acids 1-20. As another example, fragment 1-20 can be combined with fragment 50-60 to produce a single fragment of this invention having 31 amino acids (AA 10-20 and AA 50-60). Also fragments can be present in multiple numbers and in any combination in a fragment of this invention. Thus, for example, fragment 1-150 can be combined with a second fragment 1-150 and/or combined with fragment 400-500 to produce a fragment of this invention.

An active fragment of a Nkx6.1 protein of this invention can also be, for example, the repressor domain (aa 91-268), the homeodomain (aa 229-305), the activation domain (aa 306-322), the binding interference domain (aa 306-364), a fragment made up of 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, or 360 (including any number of amino acids between 5 and 360 not specifically recited herein (e.g., 32, 84, 239, etc.)) contiguous amino acids of a NKx6.1 protein of this invention that has Nkx6.1 activity.

The present invention further provides a nucleotide sequence encoding a fragment of a Nkx6.1 protein of this invention and the complement of such nucleotide sequences, as well as fragments of the nucleotide sequence encoding a NKx6.1 protein or active fragment thereof and complements thereof, for use as a probe and/or primer, for example to identify and/or amplify a Nkx6.1 protein or active fragment thereof. An example of such a fragment includes a nucleotide sequence made up of at least 5, 10, 20, 30, 40, 50, 60, 70, 80, 90, 100, 150, 200, 250, 300, 350, 400, 450, 500, 600, 700, 800, 900, or 1000 (including any number between 5 and 1000 not specifically recited herein (e.g., 43, 98, 321, etc.) contiguous nucleotides of the coding sequence of Nkx6.1. Such nucleotide sequences and/or complements thereof can also be used in the methods of this invention to suppress Nkx6.1 expression (e.g., as antisense or interfering RNA, etc.).

A “recombinant” nucleic acid is one that has been created using genetic engineering techniques that are well know in the art and as described herein.

A “recombinant polypeptide” is one that is produced from a recombinant nucleic acid.

As used herein, an “isolated” nucleic acid (e.g., an “isolated DNA” or an “isolated vector genome”) means a nucleic acid separated or substantially free from at least some of the other components of the naturally occurring organism or virus, such as for example, the cell or viral structural components or other polypeptides or nucleic acids commonly found associated with the nucleic acid.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. As used herein, the “isolated” polypeptide is at least about 25%, 50%, 60%, 70%, 75%, 80%, 85%, 90%, 95%, 97%, 98%, 99% or more pure (w/w).

As used herein an “isolated” cell is a cell that is free or substantially free from at least some of the other components of the naturally occurring organism. An “isolated” cell can be a cultured cell. Alternatively, an “isolated” cell can be a cell in a pharmaceutical composition, positioned in a selectively permeable membrane and/or in an implantable device as described herein. Further, an “isolated” cell can be a cell that has been implanted into a recipient host, e.g., in a pharmaceutical composition, a selectively permeable membrane and/or an implantable device. According to this embodiment, the cell can be derived from the host subject or can be foreign to the subject.

By the term “express,” “expresses” or “expression” of a nucleic acid coding sequence, in particular a NKX6.1 coding sequence, it is meant that the sequence is transcribed, and optionally, translated. Typically, according to the present invention, transcription and translation of the coding sequence will result in production of a NKX6.1 polypeptide or active fragment thereof.

It will be appreciated by those skilled in the art that there can be variability in the nucleic acids that encode the nkx6.1 protein of the present invention due to the degeneracy of the genetic code. The degeneracy of the genetic code, which allows different nucleic acid sequences to code for the same polypeptide, is well known in the literature (see Table 1).

TABLE 1

| Amino Acids | | | Codons |
|---|---|---|---|
| Alanine | Ala | A | GCA GCC GCG GCT |
| Cysteine | Cys | C | TGC TGT |
| Aspartic acid | Asp | D | GAC GAT |
| Glutamic acid | Glu | E | GAA GAG |
| Phenylalanine | Phe | F | TTC TTT |
| Glycine | Gly | G | GGA GGC GGG GGT |
| Histidine | His | H | CAC CAT |
| Isoleucine | Ile | I | ATA ATC ATT |
| Lysine | Lys | K | AAA AAG |
| Leucine | Leu | L | TTA TTG CTA CTC CTG CTT |
| Methionine | Met | M | ATG |
| Asparagine | Asn | N | AAC AAT |
| Proline | Pro | P | CCA CCC CCG CCT |
| Glutamine | Gln | Q | CAA CAG |
| Arginine | Arg | R | AGA AGG CGA CGC CGG CGT |
| Serine | Ser | S | AGC ACT TCA TCC TCG TCT |
| Threonine | Thr | T | ACA ACC ACG ACT |
| Valine | Val | V | GTA GTC GTG GTT |
| Tryptophan | Trp | W | TGG |
| Tyrosine | Tyr | Y | TAC TAT |

Further variation in the nucleic acid sequence can be introduced by the presence (or absence) of non-translated sequences, such as intronic sequences and 5′ and 3′ untranslated sequences.

Moreover, the isolated nucleic acids of the invention encompass those nucleic acids encoding Nkx6.1 polypeptides that have at least about 60%, 65%, 70%, 75%, 80%, 85%, 90%, 95%, 97%, 98%, 99% or 10) % amino acid sequence similarity or homology with the polypeptide sequences specifically disclosed herein (or fragments thereof) and further encode a functional Nkx6.1 or active fragment as defined herein.

As is known in the art, a number of different programs can be used to identify whether a nucleic acid or polypeptide has sequence identity, homology, or similarity to a known sequence. Sequence identity, homology and/or similarity can be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp, CABIOS 5:151-153 (1989).

Another example of a useful algorithm is the BLAST algorithm, described in Altschul et al., J. Mol. Biol. 215:403-410, (1990) and Karlin et al., Proc. Natl. Acad. Sci. USA 90:5873-5787 (1993). A particularly useful BLAST program is the WU-BLAST-2 program that was obtained from Altschul et al., Methods in Enzymology, 266:460-480 (1996). WU-BLAST-2 uses several search parameters, which are preferably set to the default values. The parameters are dynamic values and are established by the program itself depending upon the composition of the particular sequence and composition of the particular database against which the sequence of interest is being searched; however, the values can be adjusted to increase sensitivity.

An additional useful algorithm is gapped BLAST as reported by Altschul et al., (1997) Nucleic Acids Res. 25, 3389-3402.

A percentage amino acid sequence identity value can be determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The alignment can include the introduction of gaps in the sequences to be aligned. In addition, for sequences which contain either more or fewer amino acids than the polypeptides specifically disclosed herein, it is understood that in one embodiment, the percentage of sequence identity will be determined based on the number of identical amino acids in relation to the total number of amino acids. Thus, for example, sequence identity of sequences shorter than a sequence specifically disclosed herein, will be determined using the number of amino acids in the shorter sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

To modify the Nkx6.1 amino acid sequences disclosed herein or otherwise known in the art, amino acid substitutions can be based on any characteristic known in the art, including the relative similarity or differences of the amino acid side-chain substituents, for example, their hydrophobicity, hydrophilicity, charge, size, and the like. In particular embodiments, conservative substitutions (i.e., substitution with an amino acid residue having similar properties) are made in the amino acid sequence encoding Nkx6.1.

In making amino acid substitutions, the hydropathic index of amino acids can be considered. The importance of the hydropathic amino acid index in conferring interactive biologic function on a protein is generally understood in the art (see, Kyte and Doolittle, (1982) J. Mol. Biol. 157:105; incorporated herein by reference in its entirety). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, Id.), and these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is also understood in the art that the substitution of amino acids can be made on the basis of hydrophilicity. U.S. Pat. No. 4,554,101 (incorporated herein by reference in its entirety) states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±I); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

Isolated nucleic acids of this invention include RNA, DNA (including cDNAs) and chimeras thereof. The isolated nucleic acids can further comprise modified nucleotides or nucleotide analogs.

The isolated nucleic acids encoding Nkx6.1 can be associated with appropriate expression control sequences, e.g., transcription/translation control signals and polyadenylation signals.

It will be appreciated that a variety of promoter/enhancer elements can be used depending on the level and tissue-specific expression desired. The promoter can be constitutive or inducible (e.g., the metalothionein promoter or a hormone inducible promoter), depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest. In embodiments of the invention, the promoter functions in pancreatic islet beta cells. The promoter can further be “specific” for these cells and tissues (i.e., only show significant activity in the specific cell or tissue type), for example, the insulin promoter for islet beta cells; the prolactin or growth hormone promoters for anterior pituitary cells.

To illustrate, a Nkx6.1 coding sequence can be operatively associated with, for example, a cytomegalovirus (CMV) major immediate-early promoter, an albumin promoter, human insulin promoter, rat insulin promoter, mouse insulin promoter, an Elongation Factor 1-α (EF1-α) promoter, a PγK promoter, a MFG promoter, a Rous sarcoma virus (RSV) promoter, and/or a glyceraldehyde-3-phosphate promoter.

Moreover, specific initiation signals are generally required for efficient translation of inserted protein coding sequences. These translational control sequences, which can include the ATG initiation codon and adjacent sequences, can be of a variety of origins, both natural and synthetic.

In embodiments wherein the isolated nucleic acid encoding Nkx6.1 comprises an additional sequence to be transcribed, the transcriptional units can be operatively associated with separate promoters or with a single upstream promoter and one or more downstream internal ribosome entry site (IRES) sequences (e.g., the picornavirus EMC IRES sequence).

The isolated nucleic acids encoding Nkx6.1 can be incorporated into a vector, e.g., for the purposes of cloning or other laboratory manipulations, recombinant protein production, or gene delivery. Exemplary vectors include bacterial artificial chromosomes, cosmids, yeast artificial chromosomes, phage, plasmids, lipid vectors and viral vectors (described in more detail below).

In particular embodiments, the isolated nucleic acid is incorporated into an expression vector. Expression vectors compatible with various host cells are well known in the art and contain suitable elements for transcription and translation of nucleic acids. Typically, an expression vector contains an “expression cassette,” which includes, in the 5′ to 3′ direction, a promoter, a coding sequence encoding a Nkx6.1 protein or active fragment thereof operatively associated with the promoter, and, optionally, a termination sequence including a stop signal for RNA polymerase and a polyadenylation signal for polyadenylase.

Expression vectors can be designed for expression of polypeptides in prokaryotic or eukaryotic cells. For example, polypeptides can be expressed in bacterial cells such as E. coli, insect cells (e.g., in the baculovirus expression system), yeast cells or mammalian cells. Some suitable host cells are discussed further in Goeddel, Gene Expression Technology: Methods in Enzymology 185, Academic Press, San Diego, Calif. (1990). Examples of vectors for expression in yeast S. cerevisiae include pYepSecl (Baldari et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Baculovirus vectors available for expression of nucleic acids to produce proteins in cultured insect cells (e.g., Sf 9 cells) include the pAc series (Smith et al., (1983) Mol. Cell. Biol. 3:2156-2165) and the pVL series (Lucklow, V. A., and Summers, M. d. (1989) Virology 170:31-39).

Examples of mammalian expression vectors include pCDM8 (Seed, (1987) Nature 329:840) and pMT2PC (Kaufman et al. (1987), EMBO J. 6:187-195). Examples of adenovirus vectors include those described in Becker et al. (“Use of recombinant adenovirus for metabolic engineering of mammalian cells” Methods Cell Biol 43 Pt A: 161-89 (1994), the entire contents of which are incorporated herein for teachings on production and use of adenovirus vectors).

When used in mammalian cells, the expression vector's control functions are often provided by viral regulatory elements. For example, commonly used promoters are derived from polyoma, adenovirus 2, cytomegalovirus and Simian Virus 40.

In addition to the regulatory control sequences discussed above, the recombinant expression vector can contain additional nucleotide sequences. For example, the recombinant expression vector can encode a selectable marker gene to identify host cells that have incorporated the vector and/or may comprise another heterologous sequence of interest.

Vectors can be introduced into prokaryotic or eukaryotic cells via conventional transformation or transfection techniques. As used herein, the terms “transformation” and “transfection” refer to a variety of art-recognized techniques for introducing foreign nucleic acids (e.g., DNA) into a host cell, including calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, and viral-mediated transfection. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

The nucleic acids of this invention can be expressed transiently in the cell and/or can be integrated (e.g., stably integrated) into the genome of the cell. Often only a small fraction of cells (in particular, mammalian cells) integrate the foreign nucleic acid into their genome. In order to identify and select these integrants, a nucleic acid that encodes a selectable marker (e.g., resistance to antibiotics) can be introduced into the host cells along with the nucleic acid of interest. Preferred selectable markers include those that confer resistance to drugs, such as G418, hygromycin and methotrexate. Nucleic acids encoding a selectable marker can be introduced into a host cell on the same vector as that comprising the nucleic acid of interest or can be introduced on a separate vector. Cells stably transfected with the introduced nucleic acid can be identified by drug selection (e.g., cells that have incorporated the selectable marker gene will survive, while the other cells die).

### II. NUCLEIC ACID AND CELL BASED THERAPIES

The present invention is based on the unexpected discovery that an increase in Nkx6.1 activity can result in stimulation of growth of pancreatic islet beta cells and enhancement of glucose stimulated insulin secretion (GSIS). The present invention is based on the further unexpected discovery that this growth stimulation and GSIS enhancement can occur simultaneously in cells with increased Nkx6.1 activity.

Thus, the present invention provides a method of stimulating growth of a cell of this invention, e.g., pancreatic islet beta cell, comprising delivering to the cell an exogenous nucleotide sequence encoding Nkx6.1 or an active fragment thereof and/or an exogenous nucleotide sequence that increases Nkx6.1 activity in the cell.

Also provided herein is a method of enhancing glucose stimulated insulin secretion of a cell of this invention, e.g., a pancreatic islet beta cell, comprising delivering to the cell an exogenous nucleotide sequence encoding Nkx6.1 or an active fragment thereof and/or an exogenous nucleotide sequence that increases Nkx6.1 activity in the cell.

Further provided is a method of enhancing glucose stimulated insulin secretion and stimulating growth of a cell of this invention, e.g., a pancreatic islet beta cell, comprising delivering to the cell an exogenous nucleotide sequence encoding Nkx6.1 or an active fragment thereof and/or an exogenous nucleotide sequence that increases Nkx6.1 activity in the cell.

An Nkx6.1 protein of this invention can be any Nkx6.1 protein now known or later identified, including but not limited to human Nkx6.1, having the amino acid sequence (SEQ ID NO:2) and nucleotide sequence (SEQ ID NO:1) as set forth under GenBank Accession No. NM_{—}006168; rat Nkx6.1, having the amino acid and nucleotide sequence as set forth under GenBank Accession No. NM_{—}031737 and under GenBank Accession No. AF004431; mouse Nkx6.1, having the amino acid and nucleotide sequence as set forth under GenBank Accession No. NM_{—}144955; golden hamster Nkx6.1, having the amino acid and nucleotide sequence as set forth under GenBank Accession No. X81409 and zebrafish Nkx6.1, having the amino acid and nucleotide sequence as set forth under GenBank Accession No. AY437556. The sequences and information provided under these accession numbers in the GenBank database are incorporated herein by reference in their entireties.

The exogenous nucleotide sequence encoding Nkx6.1 or an active fragment thereof can be a) the nucleotide sequence of SEQ ID NO:1; b) a nucleotide sequence encoding a polypeptide comprising the amino acid sequence of SEQ ID NO:2 or an active fragment thereof; c) a nucleotide sequence that hybridizes under stringent conditions to the nucleotide sequence of (a) or (b) above and has Nkx6.1 activity; d) a nucleotide sequence having at least 65%, 70%, 75%, 80%, 85%, 90%, 95, 98%, 99% or 100% homology with a nucleotide sequence of (a), (b) or (c) above and has Nkx6.1; and e) a nucleotide sequence that differs from (a), (b), (c) or (d) above due to the degeneracy of the genetic code and encodes a polypeptide that has Nkx6.1 activity.

The term “NKx6.1 activity” as used herein refers to the activity of the wild type protein, which comprises all four of the activities of the Nkx6.1 protein, (repressor domain at aa 91-268; homeodomain at aa 229-305; activation domain at aa 306-322; and binding interference domain at aa 306-364), and/or any individual activity or combination of activities described herein or otherwise identified for the Nkx6.1 protein.

The term “stringent” as used herein refers to hybridization conditions that are commonly understood in the art to define conditions for hybridization and washing under which nucleotide sequences at least 60% (65%, 70%, 75%, 80%, 85%, 90%, 95, 97%, 98%, 99% or 100%) identical to each other typically remain hybridized to each other. Such stringent conditions are known to those skilled in the art and can be found, for example, in Current Protocols in Molecular Biology, John Wiley & Sons, N.Y. (1989), 6.3.1-6.3.6. In certain embodiments, an isolated nucleic acid molecule of the invention that hybridizes under stringent conditions to the nucleotide sequences as set forth in the Sequence Listing, or a complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

Stringency conditions can be low, high or medium, as those terms are commonly know in the art and well recognized by one of ordinary skill. A non-limiting example of stringent (i.e., high stringency) hybridization conditions are hybridization in 6×sodium chloride/sodium citrate (SSC) at about 45° C., followed by one or more washes in 0.2×SSC, 0.1% SDS at 50-65° C. Other nonlimiting examples of stringent conditions can include highly stringent (i.e., high stringency) conditions (e.g., hybridization in 0.5M NaHPO₄, 7% sodium dodecyl sulfate (SDS), 1 mM EDTA at 65° C., and washing in 0.1×SSC/0.1% SDS at 65° to 70° C.), and/or moderately stringent (i.e., medium stringency) conditions (e.g., washing in 0.2×SSC/0.1% SDS at 42° C.).

The nucleic acid of this invention can be included in a vector, which can be a viral vector. Examples of viruses that can be used as vectors of this invention include but are not limited to, adenoviruses, adeno-associated viruses, lentiviruses (e.g., human; feline), retroviruses, alphaviruses, vaccine viruses and any other virus suitable for delivering nucleic acid to a cell.

In methods of this invention wherein glucose stimulated insulin secretion is enhanced, such enhancement is relative to the amount of glucose stimulated insulin secretion in a cell lacking the exogenous Nkx6.1 nucleotide sequence and/or increased Nkx6.1 activity of this invention. Also, in methods of this invention wherein cell growth or proliferation is stimulated or enhanced, such stimulation or enhancement of growth or proliferation is relative to the amount of growth or proliferation of a cell lacking the exogenous Nkx6.1 nucleotide sequence and/or increased Nkx6.1 activity of this invention.

Thus, in some embodiments, the enhancement of GSIS in a cell and/or subject of this invention can an increase of GSIS of at least about 1%, 2%, 3%, 4%, 5%, 6%, 7%. 8%, 9%, 10%, 12%, 15%, 18%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 300%, etc., relative to the amount of GSIS in a cell lacking the exogenous Nkx6.1 nucleotide sequence and/or increased Nkx6.1 activity of this invention.

In addition, the enhancement of GSIS in a cell and/or subject of this invention can be increased by at least about 0.1 fold, 0.2 fold, 0.5 fold, 1.0 fold, 1.5 fold, 2.0 fold, 2.5 fold, 3.0 fold, 3.5 fold, 4.0 fold, 4.5 fold, 5.0 fold, 5.5 fold, 6.0 fold, 6.5 fold, 7.0 fold, 7.5 fold, 8.0 fold, 8.5 fold, 9.0 fold, 9.5 fold, 10 fold, 20 fold, 30 fold, 40 fold, 50 fold, 60 fold, 70 fold, 80 fold, 90 fold, 100 fold, etc., relative to the amount of GSIS in a cell lacking the exogenous Nkx6.1 nucleotide sequence and/or increased nkx6.1 activity of this invention.

Furthermore, the stimulation of growth or proliferation of a cell and/or a population of cells of this invention can be an increase in cell growth or proliferation (e.g. of a population of cells) of at least about 1%, 2%, 3%, 4%, 5%, 6%, 7%. 8%, 9%, 10%, 12%, 15%, 18%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 300%, etc., relative to the amount of cell growth or proliferation in a cell and/or a population of cells lacking the exogenous Nkx6.1 nucleotide sequence and/or increased Nkx6.1 activity of this invention.

In addition, the stimulation or enhancement of cell growth or proliferation of a cell and/or a population of cells of this invention can be an increase of at least about 0.1 fold, 0.2 fold, 0.5 fold, 1.0 fold, 1.5 fold, 2.0 fold, 2.5 fold, 3.0 fold, 3.5 fold, 4.0 fold, 4.5 fold, 5.0 fold, 5.5 fold, 6.0 fold, 6.5 fold, 7.0 fold, 7.5 fold, 8.0 fold, 8.5 fold, 9.0 fold, 9.5 fold, 10 fold, 20 fold, 30 fold, 40 fold, 50 fold, 60 fold, 70 fold, 80 fold, 90 fold, 100 fold, etc., relative to the amount of cell growth or proliferation in a cell and/or a population of cells lacking the exogenous Nkx6.1 nucleotide sequence and/or increased Nkx6.1 activity of this invention.

In other embodiments provided herein, the present invention includes a method of suppressing or reducing growth of a pancreatic islet beta cell, comprising delivering to the cell a nucleic acid encoding an exogenous nucleotide sequence that suppresses expression of a Nkx6.1 gene and/or suppresses Nkx6.1 activity in the cell.

Further provided is a method of suppressing glucose stimulated insulin secretion of a cell of this invention, e.g., a pancreatic islet beta cell, comprising delivering to the cell a nucleic acid encoding an exogenous nucleotide sequence that suppresses expression of a Nkx6.1 gene and/or suppresses Nkx6.1 activity in the cell.

Also provided herein is a method of suppressing growth of a cell of this invention, e.g., a pancreatic beta islet beta cell and suppressing glucose stimulated insulin secretion, comprising delivering to the cell a nucleic acid encoding an exogenous nucleotide sequence that suppresses expression of a Nkx6.1 gene and/or suppresses Nkx6.1 activity in the cell.

In methods of this invention wherein glucose stimulated insulin secretion is suppressed or reduced, such suppression or reduction is relative to the amount of glucose stimulated insulin secretion in a cell lacking the exogenous Nkx6.1 nucleotide sequence and/or increased Nkx6.1 activity of this invention. Also, in methods of this invention wherein cell growth is suppressed or reduced, such suppression or reduction of growth is relative to the amount of growth of a cell lacking the exogenous Nkx6.1 nucleotide sequence and/or increased Nkx6.1 activity of this invention.

Thus, in some embodiments, the suppression or reduction of GSIS in a cell and/or subject of this invention can be a decrease in GSIS of at least about 1%, 2%, 3%, 4%, 5%, 6%, 7%. 8%, 9%, 10%, 12%, 15%, 18%, 20%, 25%, 30%, 35%, 40%, 45%, 50%, 55%, 60%, 65%, 70%, 75%, 80%, 85%, 90%, 95%, 100%, etc., relative to the amount of GSIS in a cell lacking the exogenous Nkx6.1 nucleotide sequence and/or increased Nkx6.1 activity of this invention.

In addition, the suppression or reduction of GSIS in a cell and/or subject of this invention can be a decrease of at least about 0.1 fold, 0.2 fold, 0.5 fold, 1.0 fold, 1.5 fold, 2.0 fold, 2.5 fold, 3.0 fold, 3.5 fold, 4.0 fold, 4.5 fold, 5.0 fold, 5.5 fold, 6.0 fold, 6.5 fold, 7.0 fold, 7.5 fold, 8.0 fold, 8.5 fold, 9.0 fold, 9.5 fold, 10 fold, 20 fold, 30 fold, 40 fold, 50 fold, 60 fold, 70 fold, 80 fold, 90 fold, 100 fold, etc., relative to the amount of GSIS in a cell lacking the exogenous Nkx6.1 nucleotide sequence and/or increased nkx6.1 activity of this invention.

Furthermore, the suppression or reduction of growth of a cell and/or a population of cells of this invention can be a decrease in cell growth (e.g., of a population of cells) of at least about 1%, 2%, 3%, 4%, 5%, 6%, 7%. 8%, 9%, 10%, 12%, 15%, 18%, 20%, 25%, 30%, 35%, 40%, 45%, 50%, 55%, 60%, 65%, 70%, 75%, 80%, 85%, 90%, 95%, 100%, etc., relative to the amount of cell growth in a cell and/or a population of cells lacking the exogenous Nkx6.1 nucleotide sequence and/or increased Nkx6.1 activity of this invention.

In addition, the suppression or reduction of cell growth of a cell and/or a population of cells of this invention can be a decrease of at least about 0.1 fold, 0.2 fold, 0.5 fold, 1.0 fold, 1.5 fold, 2.0 fold, 2.5 fold, 3.0 fold, 3.5 fold, 4.0 fold, 4.5 fold, 5.0 fold, 5.5 fold, 6.0 fold, 6.5 fold, 7.0 fold, 7.5 fold, 8.0 fold, 8.5 fold, 9.0 fold, 9.5 fold, 10 fold, 20 fold, 30 fold, 40 fold, 50 fold, 60 fold, 70 fold, 80 fold, 90 fold, 100 fold, etc., relative to the amount of cell growth in a cell and/or a population of cells lacking the exogenous Nkx6.1 nucleotide sequence and/or increased Nkx6.1 activity of this invention.

A variety of nucleic acids encoding an exogenous nucleotide sequence that suppresses or reduces Nkx6.1 gene expression and/or Nkx6.1 activity in a cell or subject of this invention can be employed in the methods of this invention. Such nucleic acids include, but are not limited to antisense nucleic acids, interfering RNAs (e.g., small interfering RNAs or siRNAs), ribozymes, and any other nucleic acid that acts to suppress Nkx6.1 gene expression and/or Nkx6.1 activity in a cell or subject of this invention. Nonlimiting example of siRNAs of this invention include nucleic acids that target the following Nkx6.1 mRNA nucleotide sequences: GAGCACGCTTGGCCTATTC (bp 800-818 relative to the start codon) (SEQ ID NO:3); GGTATTCACACCACTTGGC (bp 1677-1695 relative to start codon) (SEQ ID NO:4); GTACTTGGCAGGACCAGAG (bp 780-818 relative to start codon) (SEQ ID NO:5); and AGCACAAATCCAGCGGAGG (bp 1037-1055 relative to start codon) (SEQ ID NO:6). These are examples of four target sequences that can be used for suppressing Nkx6.1 expression. The “target” sequence in these examples refers to the 19 base pairs of rat Nkx6.1 mRNA sequence that was selected for siRNA targeting with the starting position relative to the first base pair of the start codon in the Nkx6.1 mRNA. Other examples of Nkx6.1 sequences that can be targeted according to the methods of this invention include nucleotide sequences encoding the Nkx6.1 domains described herein.

Delivery of siRNAs can been accomplished through two delivery systems. The first is via recombinant adenovirus using the human H1 RNA polymerase III promoter to drive expression of an inverted repeat target sequence separated by a nine-nucleotide loop sequence (Bain et al. “An adenovirus vector for efficient RNA interference-mediated suppression of target genes in insulinoma cells and pancreatic islets of Langerhans” Diabetes 53:2190-2194 (2004)). As shown below for the examples of target sequences provided herein, the “hairpin” sequence refers to the sequence of the inverted repeat target sequence (N19-Hairpin-N19(inverted)) that is used in the adenovirus delivery system. “Duplex” sequence refers to the second delivery system—via transfection of an RNA duplex, consisting of the 19 base pairs complementary to the Nkx6.1 target sequence with a double thymine overhang and the sense and antisense RNA oligonucleotides used for that construct are shown. “Other species” indicates a 100% identity match between rat and the indicated species. “Probable species” indicates less than 100% identity but still a high probability of successful targeting. For example, Target 800-818, in addition to its use in rat cell systems, has been validated in both mouse cell lines and primary mouse islets. The hairpin (Panel A; SEQ ID NO:7) and duplex (Panel B; (SEQ ID NOS:8 and 9) configurations are shown for target 800-818 to differentiate the two systems.

Target 1677-1695:
(SEQ ID NO: 4)
GGTATTCACACCACTTGGC
Hairpin sequence:
(SEQ ID NO: 10)
GGTATTCACACCACTTGGCTTCAAGAGAGCCAAGTGGTGTGAATACC
Duplex sequence:
(SEQ ID NO: 11)
sense- GGUAUUCACACCACUUGGCtt
(SEQ ID NO: 12)
antisense- GCCAAGUGGUGUGAAUACCtt
Target 1037-1055:
(SEQ ID NO: 6)
AGCACAAATCCAGCGGAGG
Hairpin sequence:
(SEQ ID NO: 13)
AGCACAAATCCAGCGGAGGTTCAAGAGACCTCCGCTGGATTTGTGC
Duplex sequence:
(SEQ ID NO: 14)
sense-AGCACAAAUCCAGCGGAGGtt,
(SEQ ID NO: 15)
antisense-CCUCCGCUGGAUUUGUGCUtt
Probable species: mouse, human
Target 800-818:
(SEQ ID NO: 3)
GAGCACGCTTGGCCTATTC
Hairpin sequence:
(SEQ ID NO:16)
GAGCACGCTTGGCCTATTCTTCAAGAGAGAATAGGCCAAGCGTGCTC
Duplex sequence:
(SEQ ID NO: 8)
sense- GAGCACGCUUGGCCUAUUCtt,
(SEQ ID NO: 9)
antisense-GAAUAGGCCAAGCGUGCUCtt
Other species: mouse
Probable species: human, hamster
Target 780-818:
(SEQ ID NO: 5)
GTACTTGGCAGGACCAGAG
Hairpin sequence:
(SEQ ID NO: 17)
GTACTTGGCAGGACCAGAGTTCAAGAGACTCTGGTCCTGCCAAGTAC
Duplex sequence:
(SEQ ID NO: 18)
sense- GUACUUGGCAGGACCAGAGtt,
(SEQ ID NO: 19)
antisense-CUCUGGUCCUGCCAAGUACtt
Other species: mouse
Probable species: human, hamster

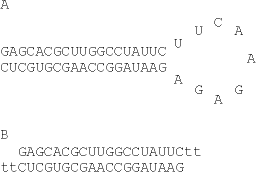

In the methods of this invention, the cell and/or population of cells of this invention can be in vitro, in vivo or ex vivo. Nonlimiting examples of a cell of this invention that can be employed in the methods and compositions of this invention include pancreatic islet beta cells, insulinoma cells (e.g., INS-1 cells including their derivatives such as the highly glucose-responsive line 832/13, HIT-T15 cells, RINr1046-38 cells, MSL-G2 cells, beta-cells expressing T-antigen, typically referred to as TC cells, including bTC-3 and bTC-6 cells, MIN6 cells), AtT-20 cells, GH1 and GH3 cells, CTG-5 cells, CTG-6 cells, primary beta-cells from all species and “artificial beta-cells,” which are non-beta-cells that have been engineered to produce insulin and, optionally, to respond to glucose and other fuels by secreting insulin.

Furthermore the cell can be from any subject that produces insulin, including but not limited to a human, cat, dog, rat, mouse, chimpanzee, pig, monkey, ape, all fish species, guinea pig, hamster, horse, sheep, goat, rabbit, etc.

In further embodiments of this invention, compositions are provided, including, for example, a composition comprising a population of cells comprising an exogenous nucleotide sequence encoding Nkx6.1 transcription factor. This population of cells can be present in a pharmaceutically acceptable carrier, thus the present invention further provides a composition comprising a population of cells comprising an exogenous nucleotide sequence encoding Nkx6.1 and a pharmaceutically acceptable carrier.

The population of cells of this invention can be contained within a biocompatible material, such as those described herein, including, but not limited to, alginate beads and microencapsulation devices.

The nucleic acids, cells, proteins and active fragments thereof can be used in methods of this invention to impart a therapeutic effect. Thus, in one embodiment, the present invention provides a method of treating diabetes in a subject (e.g., a subject in need thereof), comprising delivering to the subject a cell of this invention (e.g., a pancreatic islet beta cell) comprising an exogenous nucleotide sequence encoding Nkx6.1 or an active fragment thereof.

Also provided herein is a method of treating diabetes in a subject, comprising delivering to the subject an exogenous nucleotide sequence encoding Nkx6.1 or an active fragment thereof.

The diabetes that can be treated by the methods of this invention includes type 1 diabetes mellitus and type 2 diabetes mellitus, as well as any other diabetes-like disorder involving glucose intolerance and/or impaired insulin production or utilization.

Further provided herein is a method of increasing pancreatic islet beta cell mass in a subject, comprising delivering an exogenous nucleotide sequence encoding Nkx6.1 or an active fragment thereof to a pancreatic islet beta cell of the subject and/or delivering a cell of this invention comprising an exogenous nucleotide sequence encoding Nkx6.1 or an active fragment thereof to the subject. As used herein, an increase in cell mass includes an increase in total number of cells and/or an increase in cell volume and/or size.

Additionally provided is a method of increasing glucose stimulated insulin secretion from pancreatic islet beta cells in a subject, comprising delivering an exogenous nucleotide sequence encoding Nkx6.1 or an active fragment thereof to a pancreatic islet beta cell of the subject and/or delivering to the subject a cell comprising an exogenous nucleotide sequence encoding Nkx6.1 or an active fragment thereof.

The present invention further provides a method of increasing pancreatic islet beta cell mass and increasing glucose stimulated insulin secretion from pancreatic islet beta cells in a subject, comprising delivering an exogenous nucleotide sequence encoding Nkx6.1 or an active fragment thereof to a pancreatic islet beta cell of the subject and/or delivering to the subject a cell comprising an exogenous nucleotide sequence encoding Nkx6.1 or an active fragment thereof.

In other embodiments provided herein, the present invention includes a method of treating a disorder associated with hyperproliferation of pancreatic islet beta cells and/or hypersecretion of insulin in a subject, comprising delivering to the subject a nucleic acid comprising an exogenous nucleotide sequence that suppresses expression of a Nkx6.1 gene and/or suppresses Nkx6.1 activity. Nonlimiting examples of subjects for which this method would provide a therapeutic benefit include subjects with mutations in their sulfonylurea receptor/ATP dependent potassium channel complex proteins, resulting in constitutive hyperinsulinism and subjects with mutations in their glucokinase gene, resulting in an increase in sensitivity of glucokinase to glucose, thereby increasing the rate of glucose metabolism at low glucose and producing hyperinsulinemia.

Further provided herein is a method of decreasing pancreatic islet beta cell mass in a subject, comprising delivering to the subject a nucleic acid comprising an exogenous nucleotide sequence that suppresses expression of a Nkx6.1 gene and/or suppresses Nkx6.1 activity.

In addition, the present invention provides a method of decreasing glucose stimulated insulin secretion from pancreatic islet beta cells in a subject, comprising delivering to the subject a nucleic acid comprising an exogenous nucleotide sequence that suppresses expression of a Nkx6.1 gene and/or suppresses Nkx6.1 activity.

As noted above, a variety of nucleic acids encoding an exogenous nucleotide sequence that suppresses Nkx6.1 gene expression and/or Nkx6.1 activity in a cell or subject of this invention can be used in the methods described herein. Such nucleic acids include, but are not limited to antisense nucleic acids, interfering RNAs (e.g., small interfering RNAs or siRNAs), ribozymes, and any other nucleic acid that acts to suppress Nkx6.1 gene expression and/or Nkx6.1 activity in a cell or subject of this invention.

One approach involves the introduction of an antisense nucleotide sequence. The term “antisense nucleotide sequence,” as used herein, refers to a nucleotide sequence that is complementary to a specified DNA or RNA sequence. Antisense RNA sequences and nucleic acids that express the same can be made in accordance with conventional techniques. See, e.g., U.S. Pat. No. 5,023,243 to Tullis; U.S. Pat. No. 5,149,797 to Pederson et al.

As one example, an antisense RNA can be expressed by juxtaposition of a coding sequence for Nkx6.1 (or a portion thereof) in a reverse orientation behind a suitable promoter, such that an antisense RNA molecule is produced. This antisense construct is then introduced into the cell and, upon its expression, the production of Nkx6.1 is reduced in the cell. Alternatively, an antisense nucleotide sequence can be directly introduced into the cell by other techniques, such as electroporation. After appropriate selection to obtain cells that have stably incorporated the antisense nucleotide sequence (e.g., by stable incorporation into their genome or by stable maintenance of episomal constructs), expression of the antisense mRNA can be evaluated (for example, by hybridization to labeled sense RNA, e.g., prepared with the pGEM vector system; Promega). One can assess whether the presence of the antisense nucleotide sequence affects the production of Nkx6.1 polypeptide or Nkx6.1 activity using known techniques in the art.

Those skilled in the art will appreciate that it is not necessary that the antisense nucleotide sequence be fully complementary to the target sequence as long as the degree of sequence similarity is sufficient for the antisense nucleotide sequence to hybridize to its target and reduce production of Nkx6.1. As is known in the art, a higher degree of sequence similarity is generally required for short antisense nucleotide sequences, whereas a greater degree of mismatched bases will be tolerated by longer antisense nucleotide sequences. Alternatively stated, antisense nucleotide sequences of the invention have at least about 60%, 70%, 80%, 90%, 95%, 97%, 98% or higher sequence similarity with the Nkx6.1 coding sequence (or portions thereof) and reduce the level of Nkx6.1 polypeptide production (as defined above).

The length of the antisense nucleotide sequence (i.e., the number of nucleotides therein) is not critical as long as it binds selectively to the intended location and reduces transcription and/or translation of the target sequence (e.g., by at least about 40%, 50%, 60%, 70%, 80%, 90%, 95% or more), and can be determined in accordance with routine procedures. In general, the antisense nucleotide sequence will be from about eight, ten or twelve nucleotides in length up to about 20, 30, 50, 60 or 70 nucleotides, or longer, in length.

An antisense nucleotide sequence can be constructed using chemical synthesis and enzymatic ligation reactions by procedures known in the art. For example, an antisense nucleotide sequence can be chemically synthesized using naturally occurring nucleotides or various modified nucleotides designed to increase the biological stability of the molecules or to increase the physical stability of the duplex formed between the antisense and sense nucleotide sequences, e.g., phosphorothioate derivatives and acridine substituted nucleotides can be used. Examples of modified nucleotides which can be used to generate the antisense nucleotide sequence include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleotide sequence can be produced using an expression vector into which a nucleic acid has been cloned in an antisense orientation (i.e., RNA transcribed from the inserted nucleic acid will be of an antisense orientation to a target nucleic acid of interest).

The antisense nucleotide sequences of the invention can further include nucleotide sequences wherein at least one, or all, or the internucleotide bridging phosphate residues are modified phosphates, such as methyl phosphonates, methyl phosphonothioates, phosphoromorpholidates, phosphoropiperazidates and phosphoramidates. For example, every other one of the internucleotide bridging phosphate residues can be modified as described. In another non-limiting example, the antisense nucleotide sequence is a nucleotide sequence in which one, or all, of the nucleotides contain a 2′ loweralkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides can be modified as described. See also, Furdon et al., (1989) Nucleic Acids Res. 17:9193-9204; Agrawal et al., (1990) Proc. Natl. Acad. Sci. USA 87:1401-1405; Baker et al., (1990) Nucleic Acids Res. 18:3537-3543; Sproat et al., (1989) Nucleic Acids Res. 17:3373-3386; Walder and Walder, (1988) Proc. Natl. Acad. Sci. USA 85:5011-5015; incorporated by reference herein in their entireties for their teaching of methods of making antisense molecules, including those containing modified nucleotide bases).

RNA interference (RNAi) provides another approach for reducing Nkx6.1 activity. The interfering RNA can be directed against the Nkx6.1 coding sequence in the cell and/or any other sequence that results in a reduction in or suppression of Nkx6.1 activity.

RNAi is a mechanism of post-transcriptional gene silencing in which double-stranded RNA (dsRNA) corresponding to a coding sequence of interest is introduced into a cell or an organism, resulting in degradation of the corresponding mRNA. The RNAi effect persists for multiple cell divisions before gene expression is regained. RNAi is therefore a powerful method for making targeted knockouts or “knockdowns” at the RNA level. RNAi has proven successful in human cells, including human embryonic kidney and HeLa cells (see, e.g., Elbashir et al., Nature (2001) 411:494-8). In one embodiment, silencing can be induced in mammalian cells by enforcing endogenous expression of RNA hairpins (see Paddison et al., (2002), PNAS USA 99:1443-1448). In another embodiment, transfection of small (e.g., 21-23 nt) dsRNA specifically inhibits nucleic acid expression (reviewed in Caplen, (2002) Trends in Biotechnology 20:49-51).

The mechanism by which RNAi achieves gene silencing has been reviewed in Sharp et al. (2001) Genes Dev 15:485-490; and Hammond et al. (2001) Nature Rev Gen 2:110-119).

RNAi technology utilizes standard molecular biology methods. To illustrate, dsRNA corresponding to all or a part of a target coding sequence to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Silencing effects similar to those produced by RNAi have been reported in mammalian cells with transfection of a mRNA-cDNA hybrid construct (Lin et al., (2001) Biochem Biophys Res Commun 281:639-44), providing yet another strategy for silencing a coding sequence of interest.

In addition, silencing effects have been successfully demonstrated in pancreatic islet beta-cells (Bain et al. (2004) “An adenovirus vector for efficient RNA interference-mediated suppression of target genes in insulinoma cells and pancreatic islets of langerhans” Diabetes 53(9):2190-4; Iype et al. (2005) “Mechanism of insulin gene regulation by the pancreatic transcription factor Pdx-1: application of pre-mRNA analysis and chromatin immunoprecipitation to assess formation of functional transcriptional complexes” J Biol Chem 280(17):16798-807; Schisler et al. (2005). “The Nkx6.1 homeodomain transcription factor suppresses glucagon expression and regulates glucose-stimulated insulin secretion in islet beta cells.” Proc Natl Acad Sci USA 102(20): 7297-302; the entire contents of each of which are incorporated by reference herein).

An alternative approach for the reduction of Nkx6.1 activity is through homologous recombination. Homologous recombination relies, like antisense, on the tendency of nucleic acids to base pair with complementary sequences. In this instance, the base pairing serves to facilitate the interaction of two separate nucleic acid molecules so that strand breakage and repair can take place. In other words, the “homologous” aspect of the method relies on sequence homology to bring two complementary sequences into close proximity, while the “recombination” aspect provides for one complementary sequence to replace the other by virtue of the breaking of certain bonds and the formation of others.

Put into practice, homologous recombination can be used as follows. First, a target coding sequence is selected within the host cell. Sequences homologous to the target nucleic acid are then included in a genetic construct, along with a mutation that will render the target coding sequence inactive (e.g., a stop codon, interruption, etc.). The homologous sequences flanking the inactivating mutation are said to “flank” the mutation. Flanking, in this context, means that target homologous sequences are located both upstream (5′) and downstream (3′) of the mutation. These sequences will generally correspond to some sequences upstream and downstream of the target coding sequence. The construct is then introduced into the cell, thus permitting recombination between the cellular sequences and the construct.

It is common to include within the genetic construct a nucleotide sequence encoding a positive selectable marker to facilitate selection for recombinants. The positive selectable marker permits selection of cells that have integrated the construct into their genomic DNA by conferring resistance to the selection agent, e.g., a biostatic or biocidal drug. In addition, a heterologous nucleotide sequence (e.g., encoding a polypeptide of interest) can advantageously be included within the genetic construct and thereby be stably introduced into the cell.

Thus, using this kind of construct, it is possible, in a single recombination event, to (i) “knock out” an endogenous coding sequence, (ii) provide a selectable marker for identifying such an event, and (iii) introduce a heterologous nucleotide sequence for expression.

Another refinement of the homologous recombination approach involves the use of a “negative” selectable marker. This marker, unlike the positive selectable marker discussed above, causes death of cells that express the marker. Thus, it is used to identify undesirable recombination events. When seeking to select homologous recombinants using a selectable marker, it is sometimes difficult in the initial screening step to identify proper homologous recombinants from recombinants generated from random, non-sequence specific events. By including a negative selectable marker in the construct, but located outside of the flanking regions, one can select against many random recombination events that result in incorporation of the negative selectable marker. Homologous recombination should not introduce the negative selectable marker into the genome, as it is outside of the flanking sequences. In particular embodiments, the genetic construct also contains a nucleotide sequence encoding a positive selectable marker as described above.

Ribozymes provide still another approach for reducing or suppressing Nkx6.1 activity. Ribozymes are RNA-protein complexes that cleave nucleic acids in a site-specific fashion. Ribozymes have specific catalytic domains that possess endonuclease activity (Kim et al. (1987) Proc. Natl. Acad. Sci. USA 84:8788; Gerlach et al. (1987) Nature 328:802; Forster and Symons, (1987) Cell 49:211). For example, a large number of ribozymes accelerate phosphoester transfer reactions with a high degree of specificity, often cleaving only one of several phosphoesters in an oligonucleotide substrate (Michel and Westhof (1990) J. Mol. Biol. 216:585; Reinhold-Hurek and Shub, (1992) Nature 357:173). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, (1989) Nature 338:217). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression can be particularly suited to therapeutic applications (Scanlon et al., (1991) Proc. Natl. Acad. Sci. USA 88:10591; Sarver et al., (1990) Science 247:1222; Sioud et al., (1992) J. Mol. Biol. 223:831).

Genomic site-directed mutagenesis with oligonucleotides is yet another approach for reducing Nkx6.1 activity in the cell. Through analysis of radiation-sensitive mutants of Ustilago maydis, several genes have been characterized that participate in DNA repair (Tsukuda et al., (1989) Gene 85:335; Bauchwitz and Holloman, (1990) Gene 96:285). One such gene, REC2, encodes a protein that catalyzes homologous pairing between complementary nucleic acids and is required for a functional recombinational repair pathway (Kmiec et al., (1994) Mol. Cell. Biol. 14:7163; Rubin et al., (1994) Mol. Cell. Biol. 14:6287). In vitro characterization of the REC2 protein showed that homologous pairing was more efficient between RNA-DNA hybrids than the corresponding DNA duplexes (Kmiec et al, (1994) Mol. Cell. Biol. 14:7163; PCT Publication No. WO 96/22364). However, efficiency in pairing between DNA:DNA duplexes could be enhanced by increasing the length of the DNA oligonucleotides (Kmiec et al., (1994) Mol. Cell. Biol. 14:7163). These observations led investigators to test the use of chimeric RNA-DNA oligonucleotides (RDOs) in the targeted modification of genes in mammalian cell lines (Yoon et al. (1996) Proc. Natl. Acad. Sci. USA 93:2071; Cole-Strauss et al. (1996) Science 273:1386; PCT Publication No. WO95/15972). The RNA-DNA oligonucleotides contained self-annealing sequences such that double-hairpin capped ends are formed. This feature is believed to increase the in vivo half-life of the RDO by decreasing degradation by helicases and exonucleases. Further, the RDOs contained a single base pair that differs from the target sequence and otherwise aligns in perfect register. It is believed that the single mismatch is recognized by the DNA repair enzymes. The RDOs further contained RNA residues modified by 2′-O-methylation of the ribose sugar, making the RDO resistant to degradation by ribonuclease activity (Monia et al., (1993) J. Biol. Chem. 268:14541).

Two separate experimental systems have been used to test the use of RDOs for targeted disruption in mammalian cell lines. In one system, RDOs were used to target and correct an alkaline phosphatase cDNA that was maintained as an episomal DNA construct in Chinese hamster ovary cells. An inactive form of alkaline phosphatase was converted to a wild-type form with an efficiency of about 30% (Yoon et al., (1996) Proc. Natl. Acad. Sci. USA 93:2071). In a second system, a genetic mutation within the chromosomal DNA was targeted and corrected. A lymphoid blast cell line was derived from a patient with sickle cell disease who was homozygous for a point mutation in the beta-globin gene. The overall frequency of gene conversion from the mutant to the wild-type form was relatively high and was found to be dose-dependent on the concentration of the RDOs (Cole-Strauss et al., (1996) Science 273:1386).

As yet another approach, random integration can be used to reduce production of Nkx6.1 in a cell. Although lacking the specificity of homologous recombination, there are situations in which random integration can be used as a method of knocking out a particular endogenous coding sequence. Unlike homologous recombination, the recombination is completely random, i.e., it does not depend on or is not effected by base-pairing of complementary nucleic acid sequences. Random integration is like homologous recombination, however, in that a genetic construct, optionally containing a heterologous nucleotide sequence to be introduced into the cell and a selectable marker, integrates into the target cell genomic DNA via strand breakage and reformation.

Because of the lack of sequence specificity, the chances of any given recombinant integrating into the target coding sequence are greatly reduced. In addition, second site integrants can result in loss of expression of the heterologous coding sequence that is introduced into the cell. For example, the second locus can encode a transcription factor needed for expression of the heterologous sequence of interest, etc. As a result, it may be necessary to screen many (e.g., hundreds of thousands) of drug-resistant recombinants before a desired mutant is found. Screening can be facilitated by examining recombinants for expression of the target coding sequence using immunologic or functional tests.

It is further contemplated in this invention that small molecules and other substances that increase or suppress Nkx6.1 gene expression and/or Nkx6.1 activity can be identified and such identified compounds can be employed in the methods of this invention.

Thus, further provided herein is a method of identifying a substance having the ability to enhance Nkx6.1 activity in a cell or a population of cells, wherein the cell or the cells of the population comprise an exogenous nucleotide sequence encoding Nkx6.1, comprising:

a) measuring the amount of cell proliferation in the cell or the population of cells in the absence of the substance;

b) measuring the amount of cell proliferation in the cell or the population of cells in the presence of the substance; and

c) comparing the amount of cell proliferation of (a) with the amount of cell proliferation of (b), whereby an increase in the amount of cell proliferation of (b) identifies a substance having the ability to enhance Nkx6.1 activity in a cell or a population of cells.

In addition, the present invention provides a method of identifying a substance having the ability to suppress Nkx6.1 activity in a cell or a population of cells, wherein the cell or cells of the population comprise an exogenous nucleotide sequence encoding Nkx6.1, comprising:

a) measuring the amount of cell proliferation in the cell or the population of cells in the absence of the substance;

b) measuring the amount of cell proliferation in the population of cells in the presence of the substance; and

c) comparing the amount of cell proliferation of (a) with the amount of cell proliferation of (b), whereby a decrease in the amount of cell proliferation of (b) identifies a substance having the ability to suppress Nkx6.1 activity in a cell or a population of cells.

Also provided herein is a method of identifying a substance having the ability to enhance Nkx6.1 gene expression and/or Nkx6.1 activity in a cell comprising a nucleotide sequence encoding a promoter region of a Nkx6.1 gene operably linked to a nucleotide sequence encoding a reporter protein, comprising:

a) measuring the amount of the reporter protein produced and/or the amount of report protein activity in the cell in the absence of the substance;

b) measuring the amount of the reporter protein produced and/or the amount of reporter protein activity in the cell in the presence of the substance; and

c) comparing the amount of reporter protein produced and/or reporter protein activity of (a) with the amount of reporter protein produced and/or reporter protein activity of (b), whereby an increase in the amount of reporter protein produced and/or reporter protein activity of (b) identifies a substance having the ability to enhance Nkx6.1 gene expression and/or Nkx6.1 activity in the cell.

In further embodiments, the present invention provides a method of identifying a substance having the ability to suppress or reduce Nkx6.1 gene expression and/or Nkx6.1 activity in a cell comprising a nucleotide sequence encoding a promoter region of a Nkx6.1 gene operably linked to a nucleotide sequence encoding a reporter protein, comprising:

a) measuring the amount of the reporter protein produced and/or the amount of report protein activity in the cell in the absence of the substance;

b) measuring the amount of the reporter protein produced and/or the amount of reporter protein activity in the cell in the presence of the substance; and

c) comparing the amount of reporter protein produced and/or reporter protein activity of (a) with the amount of reporter protein produced and/or reporter protein activity of (b), whereby a decrease in the amount of reporter protein produced and/or reporter protein activity of (b) identifies a substance having the ability to reduce or suppress Nkx6.1 gene expression and/or Nkx6.1 activity in the cell.

An example of a reporter protein that can be used in the methods of this invention includes but is not limited to luciferase, beta-galactosidase, green fluorescence protein (GFP), as well as any other reporter protein now known or later developed.

A promoter region of a Nkx6.1 gene can be from any Nkx6.1 gene now known or later identified. An example of a promoter region from a human Nkx6.1 gene (SEQ ID NO:20), a mouse Nkx6.1 gene (SEQ ID NO:21) and a rat Nkx6.1 gene (SEQ ID NO:22) are included herewith as Table 2.

Substances suitable for screening according to the above methods include small molecules, natural products, peptides, nucleic acids, etc. Sources for compounds include natural product extracts, collections of synthetic compounds, and compound libraries generated by combinatorial chemistry. Libraries of compounds are well known in the art. Small molecule libraries can be obtained from various commercial entities, for example, SPECS and BioSPEC B.V. (Rijswijk, the Netherlands), Chembridge Corporation (San Diego, Calif.), Comgenex USA Inc., (Princeton, N.J.), Maybridge Chemical Ltd. (Cornwall, UK), and Asinex (Moscow, Russia). One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan et al. “Stereoselective Synthesis of Over Two Million Compounds Having Structural Features Both Reminiscent of Natural Products and Compatible with Miniaturized Cell-Based Assays” Am. Chem Soc. 120, 8565-8566, 1998; Floyd et al. Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. In certain embodiments of the invention the methods are performed in a high-throughput format using techniques that are well known in the art, e.g., in multiwell plates, using robotics for sample preparation and dispensing, etc. Representative examples of various screening methods may be found, for example, in U.S. Pat. Nos. 5,985,829, 5,726,025, 5,972,621, and 6,015,692. The skilled practitioner will readily be able to modify and adapt these methods as appropriate.

Nonlimiting examples of substances that can be used in the methods of this invention to regulate Nkx6.1 expression include glucagon-like peptide-1, extendin 4 (a glucagon-like peptide 1 analog), glucagon, glucose, fatty acids, hepatocyte growth factor, insulin, insulin-like growth factor-1, insulin-like growth factor-2, betacellulin, growth hormone and/or prolactin, individually and/or in any combination.

It is further contemplated that the present invention provides an isolated nucleotide sequence comprising, consisting essentially of and/or consisting of a Nkx6.1 promoter region. In some embodiments, the Nkx6.1 promoter region can be operably linked to a heterologous nucleotide sequence. Also provided herein are methods of expressing a heterologous nucleotide sequence in a cell and/or a subject comprising delivering to the cell and/or the subject an isolated nucleotide sequence comprising, consisting essentially of and/or consisting of a Nkx6.1 promoter region operably linked to a heterologous nucleotide sequence. The heterologous nucleotide sequence can encode a therapeutic gene product and thus, the composition comprising a Nkx6.1 promoter region operably linked to a heterologous nucleotide sequence can further comprise a pharmaceutically acceptable carrier.

A further aspect of this invention is the unexpected discovery that Nkx6.1 can act to regulate a variety of genes involved in cell cycle control. These genes include cyclin A2, cyclin B1, cyclin B2, cyclin E1, Cdk1, Cdk2, Cdc6, Cdc25a and PTTG1. Thus this invention further provides methods of modulating pancreatic islet beta-cell growth and/or modulating glucose stimulated insulin secretion in islet beta-cells by increasing or decreasing Nkx6.1 activity in islet beta cells, thereby increasing or decreasing activity of one or more genes involved in the regulation of beta-cell replication as described herein.

As noted above, the present invention is directed to methods of increasing pancreatic islet beta-cell mass and/or enhancing glucose stimulated insulin secretion in a subject, particularly a subject in need thereof as would be identified by one of ordinary skill in the art. In some embodiments, the methods of the invention comprise implanting modified cells (as described above) into such a subject. Techniques presently in use in the art for the implantation or transplantation of cells and islets will be applicable to implantation or transplantation of islets and cells engineered in accordance with the present invention.

Thus, in one embodiment, a method is provided whereby pancreatic islets are isolated from a donor (animal, human cadaver); the cells of the islets are contacted with a Nkx6.1 nucleic acid or vector of this invention and/or a substance that increases Nkx6.1 expression and/or activity in the islet cells, having the net result of expanding the number of islets cells significantly (e.g., 2-fold, 4-fold, etc. as described herein), and then delivering or introducing these the expanded, but still highly functional, islets to a subject to replace insulin, e.g., to treat type 1 or type 2 diabetes. The methods by which such islets and cells can be introduced into the subject are described herein. In particular embodiments of this invention, permanent transfection strategies (e.g., employing lentivirus vectors) can be employed in order to maintain increased Nkx6.1 expression and/or activity in the islet cells.

One representative method involves the encapsulation of cells in a biocompatible coating. In this approach, cells are entrapped in a capsular coating that protects the encapsulated cells from immunological responses, and also serves to prevent uncontrolled proliferation and spread of the cells. An exemplary encapsulation technique involves encapsulation with alginate-polylysine-alginate. In particular embodiments, capsules made by employing this technique generally contain several hundred cells and have a diameter of approximately 1 mm.

Engineered cells can be implanted using the alginate-polylysine encapsulation technique of O'Shea and Sun (1986), Diabetes 35:943, with modifications as described by Fritschy et al. (1991) Diabetes 40:37. According to this method, the engineered cells are suspended in 1.3% sodium alginate and encapsulated by extrusion of drops of the cell/alginate suspension through a syringe into CaCl₂. After several washing steps, the droplets are suspended in polylysine and rewashed. The alginate within the capsules is then reliquified by suspension in 1 ml EGTA and then rewashed with Krebs balanced salt buffer. Each capsule should contain several hundred cells and have a diameter of approximately one mm.

Implantation of encapsulated islets into animal models of diabetes by the above method has been shown to significantly increase the period of normal glycemic control, by prolonging xenograft survival compared to unencapsulated islets (O'Shea and Sun (1986), Diabetes 35:943; Fritschy, et al. (1991) Diabetes 40:37). Also, encapsulation can prevent uncontrolled proliferation of clonal cells. Capsules containing cells can be implanted (e.g., from about 500, 1,000 or 2,000 cells to about 5,000, 10,000 or 20,000 cells/animal) intraperitoneally and blood samples taken daily for monitoring of blood glucose and insulin.

An alternative approach is to seed Amicon fibers with engineered cells. The cells become enmeshed in the fibers, which are semipermeable, and are thus protected in a manner similar to the micro encapsulates (Altman et al., (1986) Diabetes 35:625).

After successful encapsulation or fiber seeding, the cells, generally approximately 1,000-10,000, can be implanted intraperitoneally, usually by injection into the peritoneal cavity through a large gauge needle (23 gauge).

A variety of other encapsulation technologies have been developed that are applicable to the practice of the present invention (see, e.g., Lacy et al., (1991), Science, 254:1782-1784; Sullivan et al., Science, 252:718-721; PCT publications WO 91/10470; WO 91/10425; WO 90/15637; WO 90/02580; WO 8901967; U.S. Pat. No. 5,011,472; U.S. Pat. No. 4,892,538; each of the foregoing being incorporated by reference herein in its entirety for teachings of encapsulation technologies). The company Cytotherapeutics has developed encapsulation technologies that are now commercially available and are of use in the application of the present invention. A vascular device has also been developed by Biohybrid, of Shrewsbury, Mass., which has application to the technology of the present invention.

With respect to implantation methods that can be employed to provide a fuel-responsive (e.g., glucose-responsive) insulin secreting capability to a mammal, particular advantages can be found in the methods recently described by Lacy et al. (1991), Science, 254:1782-1784, and Sullivan et al., (1991) Science, 252:718-721, each incorporated herein by reference in its entirety for teachings of implantation methods. These concern, firstly, the subcutaneous xenograft of encapsulated islets, and secondly, the long-term implantation of islet tissue in an “artificial pancreas” which can be connected to the vascular system as an arteriovenous shunt. These implantation methods can be advantageously adapted for use with the present invention by employing modified cells, as disclosed herein, in the place of the “islet tissue” described in these publications.

Lacy et al. ((1991), Science, 254:1782-1784) describes the encapsulation of rat islets in hollow acrylic fibers and immobilization of these in alginate hydrogel. Following intraperitoneal transplantation of the encapsulated islets into diabetic mice, normoglycemia was reportedly restored. Similar results were also obtained using subcutaneous implants that had an appropriately constructed outer surface on the fibers. The modified cells of the present invention can also be straightforwardly “transplanted” into a mammal by similar subcutaneous injection.

A biohybrid perfused “artificial pancreas,” which encapsulates islet tissue in a selectively permeable membrane, can also be employed (Sullivan et al., (1991) Science, 252:718-721). In this embodiment, a tubular semi-permeable membrane is coiled inside a protecting housing to provide a compartment for the islet cells. Each end of the membrane is then connected to an arterial polytetrafluoroethylene (PTFE) graft that extends beyond the housing and joins the device to the vascular system as an arteriovenous shunt. The implantation of such a device containing islet allografts into pancreatectomized dogs was reported to result in the control of fasting glucose levels. Grafts of this type encapsulating modified cells described herein can also be used in accordance with the present invention.

An alternate approach to encapsulation is to simply inject the modified cells into the scapular region or peritoneal cavity of diabetic mice or rats, where these cells are reported to form tumors (Sato et al., (1962) Proc. Natl. Acad. Sci. USA 48:1184). This approach is beneficial for testing the function of cells in experimental animals but is generally not applicable as a strategy for treating human diabetes.

Vector systems for genetically-modifying the secretory cells of the invention are described in more detail below. Cells that have been taken from the subject and modified ex vivo as described herein, can also be used. Methods of removing cells from subjects for delivery of nucleic acids ex vivo, followed by introduction back into the subject are known in the art (see, e.g., U.S. Pat. No. 5,399,346 for the teaching of ex vivo viral vector administration).

The present invention finds use in veterinary and medical applications. Suitable subjects include both avians and mammals, with mammals being preferred. The term “avian” as used herein includes, but is not limited to, chickens, ducks, geese, quail, turkeys and pheasants. The term “mammal” as used herein includes, but is not limited to, humans, simians and other non-human primates, bovines, ovines, caprines, equines, felines, canines, lagomorphs, rats, mice, etc. In particular embodiments, the subject is a diabetic subject (non-insulin dependent or insulin dependent diabetes mellitus), an obese subject, or a subject with impaired glucose intolerance. Human subjects include neonates, infants, juveniles, and adults. In other representative embodiments, the subject is an animal model of diabetes, obesity or impaired glucose tolerance. In other particular embodiments, the subject is a subject in need of the therapeutic methods of the invention, e.g., because the subject is diagnosed with a glucose intolerant conditions such as diabetes, is suspected of having such a condition, or is at risk of developing such a condition.

As another aspect, the present invention provides a pharmaceutical composition comprising a modified cell or cells of the invention in a pharmaceutically acceptable carrier.

In still other embodiments, the present invention provides a pharmaceutical composition comprising a protein or active fragment thereof, a nucleic acid and/or a vector of the invention in a pharmaceutically acceptable carrier.

By “pharmaceutically acceptable” it is meant a material that is not biologically or otherwise undesirable, i.e., the material can be administered to a subject without causing any undesirable biological effects such as toxicity.

A “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components (e.g., pharmaceutically acceptable carriers) include, without limitation, any of the standard pharmaceutical carriers such as phosphate buffered saline solutions, water, emulsions such as oil/water emulsion, microemulsions and various types of wetting agents, which can be sterile. In particular, it is intended that a pharmaceutically acceptable carrier be a carrier (e.g., a sterile carrier) that is formulated for administration to or delivery into a subject of this invention.

The formulations of the invention can optionally comprise medicinal agents, pharmaceutical agents, carriers, adjuvants, dispersing agents, diluents, and the like. See, e.g., Remington, The Science And Practice of Pharmacy (9^{th} Ed. 1995).

### III. DELIVERY VECTORS

The cells of the invention have been modified (i.e., genetically engineered) by introduction of an isolated nucleic acid encoding NKX6.1. In embodiments of the invention, the nucleic acid can be expressed transiently in the target cell; typically, however, the nucleic acid is stably incorporated into the target cell, for example, by integration into the genome of the cell or by persistent expression from stably maintained episomes (e.g., derived from Epstein Barr Virus).

The invention can be carried out by introducing a nucleic acid encoding a Nkx6.1 or an active fragment thereof into a cell or subject In representative embodiments of the present invention, an isolated nucleic acid encoding NKX6.1 is introduced into a cell, wherein the cell does not comprise an isolated nucleic acid encoding Nkx6.1 and/or does not overexpress a Nkx6.1 coding sequence.

It will be apparent to those skilled in the art that any suitable vector can be used to deliver the isolated nucleic acids of this invention to the target cell(s) of interest. The choice of delivery vector can be made based on a number of factors known in the art, including age and species of the target host, level and persistence of expression desired, the target cell, route of delivery, size of the isolated nucleic acid, safety concerns, and the like.

Suitable vectors include virus vectors (e.g., retrovirus, lentivirus, alphavirus; vaccinia virus; adenovirus, adeno-associated virus, or herpes simplex virus), lipid vectors, poly-lysine vectors, synthetic polyamino polymer vectors that are used with nucleic acid molecules, such as plasmids, and the like.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picornaviridae; Plasmaviridae; Prodoviridae; Polydnaviridae; Potexvirus group; Potyvirus; Poxyiridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and plant virus satellites.

Protocols for producing recombinant viral vectors and for using viral vectors for nucleic acid delivery can be found in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997).

Particular examples of viral vectors are those previously employed for the delivery of nucleic acids including, for example, retrovirus, lentivirus, adenovirus, AAV, herpes virus, and poxvirus vectors. The amount of virus delivered to a cell or a subject of this invention can vary depending on the cell type, the condition of the subject, the type of virus, the units of virus to be administered or delivered (e.g., pfu, infectious units, virus particles), etc. For example, a dosage range of an alphavirus vector can be from about 10⁴ to about 10¹⁰, 10⁵ to 10⁹, or 10⁶ to 10⁸ infectious units. An example of a dosage range of an adenovirus vector can be from about 10⁷ to 10⁹ plaque forming units (pfu) per injection, but can be as high as 10¹² pfu per injection (Crystal (1997) “Phase I study of direct administration of a replication deficient adenovirus vector containing E. coli cytosine deaminase gene to metastatic colon carcinoma of the liver in association with the oral administration of the pro-drug 5-fluorocytosine” Human Gene Therapy 8:985-1001; Alvarez and Curiel (1997) “A phase I study of recombinant adenovirus vector-mediated delivery of an anti-erbB-2 single chain (sFv) antibody gene from previously treated ovarian and extraovarian cancer patients” Hum. Gene Ther. 8:229-242; the entire contents of which are incorporated by reference herein for teachings of administration of viral vectors). Such dosages are well known in the art for a variety of viral vectors and can be readily determined by one of ordinary skill in the art for a given embodiment of this invention.

In certain embodiments of the present invention, the delivery vector is an adenovirus vector. The term “adenovirus” as used herein is intended to encompass all adenoviruses, including the Mastadenovirus and Aviadenovirus genera. To date, at least forty-seven human serotypes of adenoviruses have been identified (see, e.g., FIELDS et al., VIROLOGY, volume 2, chapter 67 (3d ed., Lippincott-Raven Publishers). In some embodiments, the adenovirus is a serogroup C adenovirus and in other embodiments, the adenovirus is serotype 2 (Ad2) or serotype 5 (Ad5).

The various regions of the adenovirus genome have been mapped and are understood by those skilled in the art (see, e.g., FIELDS et al., VIROLOGY, volume 2, chapters 67 and 68 (3d ed., Lippincott-Raven Publishers). The genomic sequences of the various Ad serotypes, as well as the nucleotide sequence of the particular coding regions of the Ad genome, are known in the art and can be accessed, e.g., from GenBank and NCBI (see, e.g., GenBank Accession Nos. J0917, M73260, X73487, AF108105, L19443, NC 003266 and NCBI Accession Nos. NC 001405, NC 001460, NC 002067, NC 00454).

Those skilled in the art will appreciate that the inventive adenovirus vectors can be modified or “targeted” as described in Douglas et al., (1996) Nature Biotechnology 14:1574; U.S. Pat. No. 5,922,315 to Roy et al.; U.S. Pat. No. 5,770,442 to Wickham et al.; and/or U.S. Pat. No. 5,712,136 to Wickham et al.

An adenovirus vector genome or rAd vector genome will typically comprise the Ad terminal repeat sequences and packaging signal. An “adenovirus particle” or “recombinant adenovirus particle” comprises an adenovirus vector genome or recombinant adenovirus vector genome, respectively, packaged within an adenovirus capsid. Generally, the adenovirus vector genome is most stable at sizes of about 28 kb to 38 kb (approximately 75% to 105% of the native genome size). In the case of an adenovirus vector containing large deletions and a relatively small heterologous nucleic acid of interest, “stuffier DNA” can be used to maintain the total size of the vector within the desired range by methods known in the art.

Normally, adenoviruses bind to a cell surface receptor (CAR) of susceptible cells via the knob domain of the fiber protein on the virus surface. The fiber knob receptor is a 45 kDa cell surface protein that has potential sites for both glycosylation and phosphorylation. (Bergelson et al., (1997), Science 275:1320-1323). A secondary method of entry for adenovirus is through integrins present on the cell surface. Arginine-Glycine-Aspartic Acid (RGD) sequences of the adenoviral penton base protein bind integrins on the cell surface.

The adenovirus genome can be manipulated such that it encodes and expresses a nucleic acid of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Representative adenoviral vectors derived from the adenovirus strain Ad type 5 d1324 or other strains of adenovirus (e.g., Ad2, Ad3, Ad7 etc.) are known to those skilled in the art.

Recombinant adenoviruses can be advantageous in certain circumstances in that they are not capable of infecting nondividing cells and can be used to infect a wide variety of cell types, including epithelial cells. Furthermore, the virus particle is relatively stable and amenable to purification and concentration, and can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., as occurs with retroviral DNA). Moreover, the carrying capacity of the adenoviral genome for foreign DNA is large relative to other delivery vectors (Haj-Ahmand and Graham (1986) J. Virol. 57:267).

In particular embodiments, the adenovirus genome contains a deletion therein, so that at least one of the adenovirus genomic regions does not encode a functional protein. For example, first-generation adenovirus vectors are typically deleted for the E1 genes and packaged using a cell that expresses the E1 proteins (e.g., 293 cells). The E3 region is also frequently deleted as well, as there is no need for complementation of this deletion. In addition, deletions in the E4, E2a, protein IX, and fiber protein regions have been described, e.g., by Armentano et al, (1997) J. Virology 71:2408, Gao et al., (1996) J. Virology 70:8934, Dedieu et al., (1997) J. Virology 71; 4626, Wang et al., (1997) Gene Therapy 4:393, U.S. Pat. No. 5,882,877 to Gregory et al. (the disclosures of which are incorporated herein in their entirety). Preferably, the deletions are selected to avoid toxicity to the packaging cell. Wang et al., (1997) Gene Therapy 4:393, has described toxicity from constitutive co-expression of the E4 and E1 genes by a packaging cell line. Toxicity can be avoided by regulating expression of the E1 and/or E4 gene products by an inducible, rather than a constitutive, promoter. Combinations of deletions that avoid toxicity or other deleterious effects on the host cell can be routinely selected by those skilled in the art.

As further examples, in particular embodiments, the adenovirus is deleted in the polymerase (pol), preterminal protein (pTP), IVa2 and/or 100K regions (see, e.g., U.S. Pat. No. 6,328,958; PCT publication WO 00/12740; and PCT publication WO 02/098466; Ding et al., (2002) Mol. Ther. 5:436; Hodges et al., J. Virol. 75:5913; Ding et al., (2001) Hum Gene Ther 12:955; the disclosures of which are incorporated herein by reference in their entireties for the teachings of how to make and use deleted adenovirus vectors for gene delivery).

The term “deleted” adenovirus as used herein refers to the omission of at least one nucleotide from the indicated region of the adenovirus genome. Deletions can be greater than about 1, 2, 3, 5, 10, 20, 50, 100, 200, or even 500 nucleotides. Deletions in the various regions of the adenovirus genome can be about at least 1%, 5%, 10%, 25%, 50%, 75%, 90%, 95%, 99%, or more of the indicated region. Alternately, the entire region of the adenovirus genome is deleted. Preferably, the deletion will prevent or essentially prevent the expression of a functional protein from that region. In general, larger deletions are preferred as these have the additional advantage that they will increase the carrying capacity of the deleted adenovirus for a heterologous nucleotide sequence of interest. The various regions of the adenovirus genome have been mapped and are understood by those skilled in the art (see, e.g., FIELDS et al., VIROLOGY, volume 2, chapters 67 and 68 (3d ed., Lippincott-Raven Publishers).

Those skilled in the art will appreciate that typically, with the exception of the E3 genes, any deletions will need to be complemented in order to propagate (replicate and package) additional virus, e.g., by transcomplementation with a packaging cell.

The present invention can also be practiced with “gutted” adenovirus vectors (as that term is understood in the art, see e.g., Lieber et al., (1996) J. Virol. 70:8944-60) in which essentially all of the adenovirus genomic sequences are deleted.

Adeno-associated viruses (AAV) have also been employed as nucleic acid delivery vectors. For a review, see Muzyczka et al. Curr. Topics in Micro. and Immunol. (1992) 158:97-129). AAV are parvoviruses and have small icosahedral virions, 18-26 nanometers in diameter and contain a single stranded genomic DNA molecule 4-5 kilobases in size. The viruses contain either the sense or antisense strand of the DNA molecule and either strand is incorporated into the virion. Two open reading frames encode a series of Rep and Cap polypeptides. Rep polypeptides (Rep50, Rep52, Rep68 and Rep78) are involved in replication, rescue and integration of the AAV genome, although significant activity can be observed in the absence of all four Rep polypeptides. The Cap proteins (VP1, VP2, VP3) form the virion capsid. Flanking the rep and cap open reading frames at the 5′ and 3′ ends of the genome are 145 basepair inverted terminal repeats (ITRs), the first 125 basepairs of which are capable of forming Y- or T-shaped duplex structures. It has been shown that the ITRs represent the minimal cis sequences required for replication, rescue, packaging and integration of the AAV genome. Typically, in recombinant AAV vectors (rAAV), the entire rep and cap coding regions are excised and replaced with a heterologous nucleic acid of interest.

AAV are among the few viruses that can integrate their DNA into non-dividing cells, and exhibit a high frequency of stable integration into human chromosome 19 (see, for example, Flotte et al. (1992) Am. J. Respir. Cell. Mol. Biol. 7:349-356; Samulski et al., (1989) J Virol. 63:3822-3828; and McLaughlin et al., (1989) J. Virol. 62:1963-1973). A variety of nucleic acids have been introduced into different cell types using AAV vectors (see, for example, Hermonat et al., (1984) Proc. Natl. Acad. Sci. USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

A rAAV vector genome will typically comprise the AAV terminal repeat sequences and packaging signal. An “AAV particle” or “rAAV particle” comprises an AAV vector genome or rAAV vector genome, respectively, packaged within an AAV capsid. The rAAV vector itself need not contain AAV genes encoding the capsid and Rep proteins. In particular embodiments of the invention, the rep and/or cap genes are deleted from the AAV genome. In a representative embodiment, the rAAV vector retains only the terminal AAV sequences (ITRs) necessary for integration, excision, replication.

Sources for the AAV capsid genes can include serotypes AAV-1, AAV-2, AAV-3 (including 3a and 3b), AAV-4, AAV-5, AAV-6, AAV-7, AAV-8, as well as bovine AAV and avian AAV, and any other virus classified by the International Committee on Taxonomy of Viruses (ICTV) as an AAV (see, e.g., BERNARD N. FIELDS et al., VIROLOGY, volume 2, chapter 69 (4th ed., Lippincott-Raven Publishers).

Because of packaging limitations, the total size of the rAAV genome will preferably be less than about 5.2, 5, 4.8, 4.6, 4.5 or 4.2 kb in size.

Any suitable method known in the art can be used to produce AAV vectors expressing the nucleic acids encoding NKX6.1 of this invention (see, e.g., U.S. Pat. No. 5,139,941; U.S. Pat. No. 5,858,775; U.S. Pat. No. 6,146,874 for illustrative methods). In one particular method, AAV stocks can be produced by co-transfection of a rep/cap vector encoding AAV packaging functions and the template encoding the AAV vDNA into human cells infected with the helper adenovirus (Samulski et al., (1989) J. Virology 63:3822).

In other particular embodiments, the adenovirus helper virus is a hybrid helper virus that encodes AAV Rep and/or capsid proteins. Hybrid helper Ad/AAV vectors expressing AAV rep and/or cap genes and methods of producing AAV stocks using these reagents are known in the art (see, e.g., U.S. Pat. No. 5,589,377; and U.S. Pat. No. 5,871,982, U.S. Pat. No. 6,251,677; and U.S. Pat. No. 6,387,368). Preferably, the hybrid Ad of the invention expresses the AAV capsid proteins (i.e., VP1, VP2, and VP3). Alternatively, or additionally, the hybrid adenovirus can express one or more of AAV Rep proteins (i.e., Rep40, Rep52, Rep68 and/or Rep78). The AAV sequences can be operatively associated with a tissue-specific or inducible promoter.

The AAV rep and/or cap genes can alternatively be provided by a packaging cell that stably expresses the genes (see, e.g., Gao et al., (1998) Human Gene Therapy 9:2353; Inoue et al., (1998) J. Virol. 72:7024; U.S. Pat. No. 5,837,484; WO 98/27207; U.S. Pat. No. 5,658,785; WO 96/17947).

Another vector for use in the present invention comprises Herpes Simplex Virus (HSV). Herpes simplex virions have an overall diameter of 150 to 200 nm and a genome consisting of one double-stranded DNA molecule that is 120 to 200 kilobases in length. Glycoprotein D (gD) is a structural component of the HSV envelope that mediates virus entry into host cells. The initial interaction of HSV with cell surface heparin sulfate proteoglycans is mediated by another glycoprotein, glycoprotein C (gC) and/or glycoprotein B (gB). This is followed by interaction with one or more of the viral glycoproteins with cellular receptors. It has been shown that glycoprotein D of HSV binds directly to Herpes virus entry mediator (HVEM) of host cells. HVEM is a member of the tumor necrosis factor receptor superfamily (Whitbeck et al., (1997), J. Virol.; 71:6083-6093). Finally, gD, gB and the complex of gH and gL act individually or in combination to trigger pH-independent fusion of the viral envelope with the host cell plasma membrane. The virus itself is transmitted by direct contact and replicates in the skin or mucosal membranes before infecting cells of the nervous system for which HSV has particular tropism. It exhibits both a lytic and a latent function. The lytic cycle results in viral replication and cell death. The latent function allows for the virus to be maintained in the host for an extremely long period of time.

HSV can be modified for the delivery of nucleic acids to cells by producing a vector that exhibits only the latent function for long-term gene maintenance. HSV vectors are useful for nucleic acid delivery because they allow for a large DNA insert of up to or greater than 20 kilobases; they can be produced with extremely high titers; and they have been shown to express nucleic acids for a long period of time in the central nervous system as long as the lytic cycle does not occur.

In other embodiments of the present invention, the delivery vector of interest is a retrovirus or lentivirus. Retroviruses and lentiviruses normally bind to a virus-specific cell surface receptor, e.g., CD4 (for HIV); CAT (for MLV-E; ecotropic Murine leukemic virus E); RAM1/GLVR2 (for murine leukemic virus-A; MLV-A); GLVR1 (for Gibbon Ape leukemia virus (GALV) and Feline leukemia virus B (FeLV-B)). The development of specialized cell lines (termed “packaging cells”) which produce only replication-defective retroviruses and lentiviruses has increased the utility of retroviruses and lentiviruses for gene therapy, and defective retroviruses and lentiviruses are characterized for use in gene transfer for gene therapy purposes (Miller, (1990) Blood 76:271; Blömer et al. “Highly efficient and sustained gene transfer in adult neurons with a lentivirus vector” J Virol. 71(9):6641-6649 (1997)). A replication-defective retrovirus or lentivirus can be packaged into virions that can be used to infect a target cell through the use of a helper virus by standard techniques. Retrovirus and lentivirus vectors can also be pseudotyped to express proteins on the virus particle surface that have affinity for cell surface receptors on particular cell types, thereby allowing for targeted delivery of the viral vector to a specific cell type.

Yet another suitable vector is a poxvirus vector. These viruses are very complex, containing more than 100 proteins, although the detailed structure of the virus is presently unknown. Extracellular forms of the virus have two membranes while intracellular particles only have an inner membrane. The outer surface of the virus is made up of lipids and proteins that surround the biconcave core. Poxviruses are antigenically complex, inducing both specific and cross-reacting antibodies after infection. Poxvirus receptors are not presently known, but it is likely that there exists more than one given the tropism of poxvirus for a wide range of cells. Poxvirus gene expression is well studied due to the interest in using vaccinia virus as a vector for expression of nucleic acids.

In addition to viral transfer methods, such as those illustrated above, non-viral methods can also be employed. Many non-viral methods of nucleic acid transfer rely on normal mechanisms used by mammalian cells for the uptake and intracellular transport of macromolecules. In particular embodiments, non-viral nucleic acid delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present invention. Naked plasmids can be introduced into muscle cells by injection into the tissue. Expression can extend over many months, although the number of positive cells is typically low (Wolff et al., (1989) Science 247:247). Cationic lipids have been demonstrated to aid in introduction of nucleic acids into some cells in culture (Feigner and Ringold, (1989) Nature 337:387). Injection of cationic lipid plasmid DNA complexes into the circulation of mice has been shown to result in expression of the DNA in lung (Brigham et al., (1989) Am. J. Med. Sci. 298:278). One advantage of plasmid DNA is that it can be introduced into non-replicating cells.

In a representative embodiment, a nucleic acid molecule (e.g., a plasmid) can be entrapped in a lipid particle bearing positive charges on its surface and, optionally, tagged with antibodies against cell surface antigens of the target tissue (Mizuno et al., (1992) No Shinkei Geka 20:547; PCT publication WO 91/06309; Japanese patent application 1047381; and European patent publication EP-A-43075).

Liposomes that consist of amphiphilic cationic molecules are useful non-viral vectors for nucleic acid delivery in vitro and in vivo (reviewed in Crystal, Science 270: 404-410 (1995); Blaese et al., Cancer Gene Ther. 2: 291-297 (1995); Behr et al., Bioconjugate Chem. 5: 382-389 (1994); Remy et al., Bioconjugate Chem. 5: 647-654 (1994); and Gao et al., Gene Therapy 2: 710-722 (1995)). The positively charged liposomes are believed to complex with negatively charged nucleic acids via electrostatic interactions to form lipid:nucleic acid complexes. The lipid:nucleic acid complexes have several advantages as nucleic acid transfer vectors. Unlike viral vectors, the lipid:nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency. A number of publications have demonstrated that amphiphilic cationic lipids can mediate nucleic acid delivery in vivo and in vitro (Feigner et al., Proc. Natl. Acad. Sci. USA 84: 7413-17 (1987); Loeffler et al., Methods in Enzymology 217: 599-618 (1993); Feigner et al., J. Biol. Chem. 269: 2550-2561 (1994)).

Several groups have reported the use of amphiphilic cationic lipid:nucleic acid complexes for in vivo transfection both in animals and in humans (reviewed in Gao et al., Gene Therapy 2: 710-722 (1995); Zhu et al., Science 261: 209-211 (1993); and Thierry et al., Proc. Natl. Acad. Sci. USA 92: 9742-9746 (1995)). U.S. Pat. No. 6,410,049 describes a method of

preparing cationic lipid:nucleic acid complexes that have a prolonged shelf life.

In other embodiments, delivery of the nucleic acids of this invention to cells, especially to pancreatic islet cells can be carried out according to methods described in the Examples below, referred to as ultrasound microbubble destruction technology. Basically, nucleic acids (e.g., plasmids) and/or viruses are encapsulated in liposomic microbubbles that are introduced into a subject. The bubbles are destroyed in the area of a specific target tissue (e.g., pancreas) by application of targeted ultrasound.

Having now described the invention, the same will be illustrated with reference to certain examples, which are included herein for illustration purposes only, and which are not intended to be limiting of the invention.

### EXAMPLES

### Example 1

### Nkx6.1 Suppresses Glucagon Expression and Regulates Glucose Stimulated Insulin Secretion in Islet Beta Cells

Preparation and use of recombinant adenoviruses. INS-1-derived cell lines were prepared and cultured as previously described (3, 5). The cDNA encoding hamster Nkx6.1 (6) was used to prepare a recombinant adenovirus (AdCMV-Nkx6.1) by previously described methods (7, 8). A virus containing the bacterial β-galactosidase gene (AdCMV-βGAL) was used as a control (9). Viruses were purified using the ADENO-X purification kit (BD Biosciences), and used to treat cell lines at multiplicities of infection (MOI) ranging from 10-2500 for 2 h. Assays and analyses were undertaken 24-48 h later.

Small interfering RNA sequences (siRNAs) corresponding to rat Nkx6.1 (GenBank accession number AF004431; GAGCACGCTTGGCCTATTC; SEQ ID NO:3) and rat Pdx1 (GenBank accession number NM_{—}022852; GAAAGAGGAAGATAAGAAA; SEQ ID NO:23) were cloned into vector EH006, and used for construction of Ad-siNkx6.1 and Ad-siPdx1 recombinant adenoviruses, respectively, by described methods (10). Adenoviruses containing siRNA sequences corresponding to the Photinus pyralis luciferase gene, GL2 (11; Ad-siLuc), or a random siRNA sequence (GAGACCCTATCCGTGATTA; SEQ ID NO:24) (Ad-siRNAcontrol) were used as controls. These viral stocks were used to treat cell lines at an MOI of 20 for 18 h. Assays and analyses were undertaken 96 h later.

Glucose-Stimulated Insulin Secretion, Insulin and Glucagon Content.

Cells were aliquoted in 12-well plates, treated with the various recombinant adenoviruses, and grown in culture medium containing 11 mM glucose. Insulin secretion was measured by static incubation as described (3) following a switch to culture medium containing 5 mM glucose for 12 h, using basal and stimulatory glucose concentrations as indicated herein. For measurements of insulin and glucagon content, cells were extracted with 1 M acetic acid, 0.1% BSA. Media and extract samples were analyzed for insulin or glucagon concentrations with the insulin COAT-A-COUNT kit (Diagnostic Products, Los Angeles, Calif.) (13, 14), or a glucagon kit (Linco Research Inc.).

Semiquantitative Multiplex-PCR and Real-Time PCR Measurements of RNA Levels.

Total RNA was isolated and purified using TRizol reagent (Invitrogen) or RNeasy (Qiagen) and treated with DNase to avoid genomic contamination. Superscript II (Invitrogen) or iScript (BioRad) was used for first strand synthesis of cDNA using 0.5-1.0 μg of RNA.

Semi-quantitative multiplex-PCR was performed as described previously (15). Briefly, primers were optimized to yield products between 160-280 bp, and reactions were carried out at cycles in the exponential range of product formation (between 16-24 cycles, dependent on target) and separated on 0.4 mm, 7 M urea, 6% polyacrylamide gels. Images were acquired using a Storm phosphor-imager (Amersham Biosciences). Standard band/volume analysis using local background correction was used to quantify PCR products. Candidate gene expression profiling in the various cell lines was performed with primers specific for islet hormones (insulin, glucagon, somatostatin), enzymes and transporters (glucose-6-phosphatase, glucokinase, GLUT2, hexokinases 1 and 2, and glyceraldehyde dehydrogenase), transcription factors (Brn4, E47, HNF-1α, IB1, NeuroD, Nkx2.2, Nkx6.1, Pax4, Pdx1) and glycogen targeting subunits (PTG, G_{L}). Other studies of the effects of modulation of Nkx6.1 levels involved measurements of additional target genes and loading controls, including α-tubulin, G6PDH, Pax6, HNF3α (FOXO1), ratNkx6.1, hamster Nkx6.1, and total Nkx6.1 (rat+hamster).

Real-time PCR reactions were performed with 20 ng of cDNA using the ABI PRISM® 7000 sequence detection system, software and reagents (16).

Electrophoretic Mobility Shift (EMSA) and Chromatin Immunoprecipitation (ChIP) Assays.

EMSA buffers and procedures were as described (17). 5 μg of nuclear extract protein was added to each EMSA reaction. Where supershift assays were performed, 1 μl of undiluted anti-Nkx6.1 or anti-Pdx1 antisera was also added. EMSA was performed with an oligonucleotide corresponding to the G1 element of the rat glucagon promoter: 5′-GAACAAAACCCCATTATTTACAGATGAGAA-3′ (SEQ ID NO:25) (top strand shown).

ChIP assays were performed as detailed previously (18, 19). Antisera used in the immunoprecipitations were either anti-Nkx6.1 antiserum or normal rabbit serum. Each ChIP assay was quantitated in triplicate by real-time PCR for recovery of either the rat glucagon or myoD1 promoters. Forward and reverse primers used to amplify the glucagon gene (−1 bp to −279 bp relative to the transcriptional start site) were 5′-GGATCCTTCAGAGAGCTGAATAG (SEQ ID NO:26) and 5′-GGCAAGCTTCACCAGGGTGCTGTG (SE ID NO:27). Forward and reverse primers used to amplify the myoD1 gene were 5′-CCACTTCGTCCTTGGCTCAAC (SEQ ID NO:28) and 5′-GGGATACCAGGCACAGCATAGG (SEQ ID NO:29).

Immunoblot Analysis.

Aliquots of 5.0 μg of nuclear extract protein were resolved by SDS polyacrylamide electrophoresis and transferred to polyvinylidene difluoride (PVDF). Membranes were incubated with anti-Nkx6.1 (1:4000) or anti-Pdx1 (1:10,000) antibodies overnight at four degrees, or anti-γ-tubulin (1:10,000 Sigma) antibody for 3 h at room temperature. Antibodies were detected using appropriate HRP-linked secondary antibodies and visualized using ECL Advance (Amersham) on a Versadoc 5000 (Biorad).

Transient Transfection Assays.

αTC1.6 cells were aliquoted into 6-well plates at a density of 7.5×10⁵ cells per well one day before transfection. A total of 2 μg of plasmid DNA (consisting of 1.0 μg of a luciferase reporter plasmid under control of the proximal 450 bp of the rat glucagon promoter (pFoxLucGlucagon), 0.25 μg of pBAT12.Nkx6.1 or pBAT12 lacking a cDNA insert, and the balance consisting of a CMV promoter-driven β-galactosidase reporter plasmid) was mixed with 6 ml of Transfast® reagent (Promega), and transfections performed according to the manufacturer's protocol. Additional control experiments utilized plasmids with luciferase under control of thymidine kinase, CMV, or no promoter in place of 1 μg pFoxLucGlucagon. Cells were harvested 48 hr after transfection, and luciferase activities were measured using a commercially available assay kit (Promega) and an FB15 luminometer (Zylux).

Islet Isolation.

Islets from male Wistar rats weighing approximately 250 g were isolated via pancreatic perfusion as described (20) using the Liberase R1 enzyme (Roche), and GSIS assays were performed as described (21).

Statistical Methods.

Statistical significance was determined using a two-tailed Student's t-Test.

Glucose-Stimulated Insulin Secretion in INS-1-Derived Cell Lines.

GSIS was measured in six independent INS-1-derived cell lines (3, 5) at 1 or 20 mM glucose in a 2-h static incubation assay. Four of the cell lines were poorly glucose responsive (lines 834/105, 834/112, 832/1, 832/2; average responses of 2.4-fold, 2.1-fold, 2.2-fold, and 3.8-fold as glucose was raised from 1 to 20 mM, respectively) whereas two cell lines were robustly glucose responsive (lines 833/15, 832/13; average responses of 21-fold and 30-fold, respectively) These six lines were used for subsequent experiments.

Candidate Gene Screen.

The levels of mRNA encoding a panel of twenty transcription factors, metabolic enzymes, or hormones as described herein thought to serve either as markers or mediators of β-cell differentiation were measured by semi-quantitative multiplex-PCR. Among these, glucagon, Nkx2.2 and Nkx6.1 were differentially expressed in the six INS-1-derived cell lines. The expression pattern for the gene encoding glucagon identified new subclasses of INS-1-derived cell lines. Thus, a subset of the poorly glucose responsive cell lines (834/105 and 834/112) was found to contain substantial quantities of the mRNA encoding glucagon, and are hereafter referred to as Class 1 cells (Class 1 cells have more glucagon and Nkx2.2 mRNA, respectively, than the other two classes of cells, with P<0.008). Two other poorly glucose responsive cell lines (832/1 and 832/2) contained little glucagon transcript, and are hereafter referred to as Class 2 cells (Class 2 cells have more Nkx6.1 mRNA than Class 1 cells, with P<0.05). Finally, the two robustly glucose responsive cell lines (832/13 and 832/15) also contained very low levels of glucagon mRNA, in keeping with their well-differentiated phenotype, and are hereafter referred to as Class 3 cells (Class 3 cells have more Nkx6.1 mRNA than the other two classes, with P<0.001).

The other two differentially expressed candidate genes emerging from this study are members of the Nkx transcription factor family. Nkx2.2 was most highly expressed in the glucagon positive, poorly glucose responsive Class 1 cells (relative RNA levels of 3.3:1.0:1.0 in Class 1:Class 2 and Class 3). Interestingly, the inverse pattern was seen for Nkx6.1, which was most abundant in Class 3 cells and least abundant in Class 1 cells (relative RNA levels of 1.0:2.6:6.4 in Class 1:Class 2:Class 3).

Expression of Nkx6.1 in Class 1 Cells Suppresses Glucagon Gene Expression.

The expression pattern of Nkx6.1 described above suggests a potential role for this transcription factor in regulation of glucagon expression and/or other differentiated functions of the β-cell such as glucose stimulated insulin secretion (GSIS). Class I cells were treated with AdCMV-Nkx6.1 or AdCMV-βGAL and semiquantitative multiplex PCR analysis of hamster Nkx6.1 and glucagon mRNAs (with α-tubulin as an internal control) and immunoblot analysis of Nkx6.1 protein levels were carried out. Treatment of the Class 1 cell line 834/105 with AdCMV-Nkx6.1 adenovirus resulted in increases in Nkx6.1 mRNA and protein. Moreover, overexpression of Nkx6.1 in these cells suppressed glucagon mRNA levels in a dose-dependent manner. Also, at a dose of AdCMV-Nkx6.1 virus that caused a 50% decrease in glucagon mRNA over 24 h, a 71% decrease in glucagon peptide content was observed 48 h after viral treatment (134±24 versus 39±3 ng glucagon/mg protein in AdCMV-βGAL versus AdCMV-Nkx6.1-treated cells, respectively; p=0.0014).

Importantly, at a dose of AdCMV-Nkx6.1 adenovirus that caused a 62% suppression of glucagon mRNA levels, no effect on expression of prominent β-cell transcription factors such as Pdx1, IB1, E47, HNF-1α, Nkx2.2, and endogenous rat Nkx6.1, or PAX4, PAX6 or HNF3α (FOXO1) were observed. Overexpression of Nkx6.1 also did not affect insulin mRNA levels. Overexpression of Nkx6.1 did cause a small (10%), but statistically significant decrease in expression of NeuroD (p=0.049). These data suggest that Nkx6.1 overexpression is largely sufficient to suppress glucagon gene expression in INS-1-derived cell lines.

Nkx6.1 Interacts with the Glucagon Promoter and Suppresses its Function.

To further investigate the mechanism of Nkx6.1-mediated suppression of glucagon expression, experiments were conducted to examine the ability of the transcription factor to interact with the G1 element of the rat glucagon promoter in electromobility shift assays (EMSAs), since the G1 element contains a potential Nkx6.1 binding site (5′-TAAT-3′) (6, 22).

Nuclear extracts were prepared from 832/13 cells treated with AdCMV-Nkx6.1 or no virus and mixed with a radiolabeled oligonucleotide corresponding to the G1 element of the glucagon promoter. Before gel electrophoresis, samples were untreated or treated with anti-Nkx6.1 or anti-Pdx1 antibodies to cause a supershift of the specific Nkx6.1/G1 or Pdx1/G1 complexes.

Nuclear extracts from Class 3 cells contain significant Nkx6.1 protein, as demonstrated by supershift of a specific protein from the G1/nuclear extract complex in response to treatment with an Nkx6.1-specific antibody. Adenovirus-mediated overexpression of Nkx6.1 caused a large increase in the amount of super-shifted protein, demonstrating that the overexpressed hamster Nkx6.1 protein binds to elements found within the rat glucagon promoter.

To assess the functional consequence of interaction of Nkx6.1 with the glucagon promoter, co-transfection studies were performed in the α-cell line αTC1.6. These cells were cotransfected with the pBAT12.Nkx6.1 plasmid expressing Nkx6.1 and plasmids containing the luciferase reporter under control of the glucagon promoter, CMV promoter, TK promoter, or no promoter. Luciferase activity is expressed as fold repression relative to activity in cells cotransfected with the glucagon/luciferase promoter construct and the pBAT12 plasmid lacking a cDNA insert (empty vector).

Overexpression of Nkx6.1 caused a 9.4±1.1-fold repression of the activity of a glucagon promoter-driven luciferase reporter relative to control cells transfected with empty vector. By contrast, repression of control reporters (promoterless, thymidine kinase promoter, and CMV promoter) by overexpressed Nkx6.1 was in the 1.5- to 2.5-fold range, suggesting that repression of the glucagon promoter-driven luciferase reporter by Nkx6.1 is specific. Moreover, adenovirus-mediated expression of Nkx6.1 caused a 58% suppression of endogenous glucagon mRNA in αTC1.6 cells, demonstrating that Nkx6.1 can suppress expression of an intact glucagon gene.

These studies suggest but do not prove that Nkx6.1 binds directly to the endogenous glucagon promoter in β-cells. To address this issue, chromatin immunoprecipitation (ChIP) assays were performed. Nkx6.1 is present at relatively low abundance in β-cells, so as a prelude to these studies, multiple cell lines with robust GSIS were screened for their endogenous levels of Nkx6.1. Line 832/3, previously identified as a line with robust GSIS (3) contained 4.8 times more Nkx6.1 mRNA than lines 832/13 or 832/15 characterized earlier, and glucagon expression was virtually undetectable in these cells.

Immunoprecipitation reactions were performed with nonspecific rabbit antiserum or anti-Nkx6.1 antibody, followed by real-time PCR analysis of associated DNA with primers specific for the glucagon or myoD genes. Data are expressed as fold increase in recovery of input glucagon or myoD DNA by immunoprecipitation with the respective antibodies.

A significantly larger fraction of input glucagon promoter was recovered (2.5 times more, p=0.001) in nuclear extract samples from 832/3 cells immuno-precipitated with anti-Nkx6.1 antibody compared to samples treated with non-specific rabbit serum. Moreover, the anti-Nkx6.1 antibody did not preferentially immunoprecipitate a control promoter (myoD). These data demonstrate direct interaction of Nkx6.1 with the glucagon promoter in living β-cells.

Suppression of Nkx6.1 or Pdx1 Expression Activates Glucagon Expression.

Recombinant adenoviruses containing small interfering RNAs specific for Nkx6.1 or Pdx1 (Ad-siNkx6.1 and Ad-siPdx1) were prepared and used as reagents to suppress the expression of the individual transcription factors in Class 3 cells (832/13).

In these experiments, 832/13 cells were treated with Ad-siNkx6.1, Ad-siPdx1 or Ad-siRNAcontrol, and glucagons, Nkx6.1, Pdx1 and GLUT2 mRNA levels were analyzed by real-time PCR. Data were normalized to the levels in Ad-siRNAcontrol-treated cells.

Treatment of these cells with Ad-siNkx6.1 resulted in suppression of Nkx6.1 mRNA levels by 65%, whereas treatment with Ad-siPdx1 caused an 85% decrease in Pdx1 mRNA content. Moreover, treatment of Class 3 cells with Ad-siNkx6.1 caused near-complete extinction of the supershifted Nkx6.1 band detected by EMSA analysis with the glucagon promoter G1 element probe. Similarly, treatment of 832/13 cells with the Ad-siPdx1 adenovirus resulted in strong suppression of an anti-Pdx1 antibody-supershifted band.

The level of Nkx6.1 suppression demonstrated above resulted in a 2-fold increase in glucagon mRNA levels, further supporting a direct role for Nkx6.1 in control of glucagon expression in β-cells. Interestingly, Pdx1 suppression caused a much larger increase (12-fold) in glucagon expression than achieved by Nkx6.1 suppression. Pdx1 suppression resulted in modest decreases in Nkx6.1 mRNA levels, and in Nkx6.1 protein levels as judged by EMSA analyses. Conversely, suppression of Nkx6.1 by RNAi treatment had no effect on Pdx1 mRNA or protein levels. These data indicate that Nkx6.1 and Pdx1 have independent effects on glucagon expression in β-cells.

Nkx6.1 suppression causes impairment of GSIS in Class 3 cells. 832/13 cells were treated with Ad-siNkx6.1, Ad-siPdx1 or Ad-siRNAcontrol, followed by assay of insulin secretion in response to 3 or 15 mM glucose. Insulin secretion was expressed as microunits per ml per every 2 h.

Ad-siNkx6.1 suppressed Nkx6.1 mRNA levels by 65% in 832/13 cells. Suppression of Nkx6.1 caused a significant impairment in GSIS, with a decline from a 13.9-fold response in cells treated with the Ad-siRNA control virus to a 3.7-fold response in cells treated with Ad-siNkx6.1. Similar effects of Nkx6.1 suppression were observed in a second Class 3 cell line, 832/3. In contrast, suppression of Pdx1 expression caused a decrease in both basal and stimulated insulin secretion, but did not affect the fold response. The differential effects of Nkx6.1 and Pdx1 suggests that these transcription factors control distinct sets of genes involved in insulin secretion. Consistent with this, suppression of Pdx1 caused a 63% decrease, whereas Nkx6.1 suppression caused a 2.3-fold increase, in GLUT-2 mRNA levels. Neither Pdx-1 suppression nor Nkx6.1 suppression affected insulin content (16.8±3.5, 14.3±1.9, and 15.5±3.5 μU insulin/μg protein in Ad-siRNAcontrol-, Ad-siNkx6.1-, and Ad-siPdx1-treated cells, respectively).

Studies were also carried out to investigate whether increasing the levels of Nkx6.1 in Class 1 or Class 2 cells would result in improved GSIS in these lines. AdCMV-Nkx6.1 was used to raise Nkx6.1 mRNA levels in Class 1 or Class 2 cells to a level approximating the endogenous levels of Class 3 cells. This maneuver did not improve GSIS relative to AdCMV-βGAL-treated Class 1 or Class 2 cells. Overexpression of Nkx6.1 in Class 1 or Class 2 cells to levels higher than endogenous levels in Class 3 cells also had no effect on GSIS.

Suppression of Nkx6.1 Expression Impairs GSIS in Rat Islets.

To determine whether the findings of a role of Nkx6.1 in control of GSIS in INS-1-derived cell lines also applies to normal rat islets, the Ad-siNkx6.1 virus was used to suppress Nkx6.1 mRNA levels by 46% in primary cells. This maneuver resulted in a decrease in GSIS from 7.1±0.2-fold in Ad-siRNAcontrol-treated islets to 5.2±0.5-fold in Ad-siNkx6.1-treated islets (p=0.012), and a 56% decrease in insulin secretion at stimulatory glucose (16.7 mM). Consistent with the findings in the INS-1-derived cell lines, suppression of Nkx6.1 did not affect insulin content in the primary islet experiments.

### References for Example 1

1. Shapiro, A. M., Lakey, J. R., Ryan, E. A., Korbutt, G. S., Toth, E., Warnock, G. L., Kneteman, N. M. & Rajotte, R. V. (2000) N Engl J Med 343, 230-8.
2. Lebovitz, H. E. (2004) in Diabetes Mellitus, ed. LeRoith, D., Taylor, S. I., Olefsky, J. M. (Lippincott Williams & Wilkins, Philadelphia), pp. 1107-1138.
3. Hohmeier, H. E., Mulder, H., Chen, G., Henkel-Rieger, R., Prentki, M. & Newgard, C. B. (2000) Diabetes 49, 424-30.
4. Asfari, M., Janjic, D., Meda, P., Li, G., Halban, P. A. & Wollheim, C. B. (1992) Endocrinology 130, 167-78.
5. Chen, G., Hohmeier, H. E., Gasa, R., Tran, V. V. & Newgard, C. B. (2000) Diabetes 49, 562-70.
6. Mirmira, R. G., Watada, H. & German, M. S. (2000) J Biol Chem 275, 14743-51.
7. Gomez-Foix, A. M., Coats, W. S., Baque, S., Alam, T., Gerard, R. D. & Newgard, C. B. (1992) J Biol Chem 267, 25129-34.
8. Becker, T. C., Noel, R. J., Coats, W. S., Gomez-Foix, A. M., Alam, T., Gerard, R. D. & Newgard, C. B. (1994) Methods Cell Biol 43 Pt A, 161-89.
9. Herz, J. & Gerard, R. D. (1993) Proc Natl Acad Sci USA 90, 2812-6.
10. Bain, J. R., Schisler, J. C., Takeuchi, K., Newgard, C. B. & Becker, T. C. (2004) Diabetes 53, 2190-4.
11. Elbashir, S. M., Harborth, J., Lendeckel, W., Yalcin, A., Weber, K. & Tuschl, T. (2001) Nature 411, 494-8.
12. Bewig, B. & Schmidt, W. E. (2000) Biotechniques 28, 870-3.
13. Clark, S. A., Quaade, C., Constandy, H., Hansen, P., Halban, P., Ferber, S., Newgard, C. B. & Normington, K. (1997) Diabetes 46, 958-67.
14. Hohmeier, H. E., BeltrandelRio, H., Clark, S. A., Henkel-Rieger, R., Normington, K. & Newgard, C. B. (1997) Diabetes 46, 968-77.
15. Jensen, J., Serup, P., Karlsen, C., Nielsen, T. F. & Madsen, O. D. (1996) J Biol Chem 271, 18749-58.
16. An, J., Muoio, D. M., Shiota, M., Fujimoto, Y., Cline, G. W., Shulman, G. I., Koves, T. R., Stevens, R., Millington, D. & Newgard, C. B. (2004) Nat Med 10, 268-74.
17. German, M. S., Moss, L. G., Wang, J. & Rutter, W. J. (1992) Mol Cell Biol 12, 1777-88.
18. Chakrabarti, S K, Francis, J, Ziesmann, S M, Garmey, J C, and Mirmira, R G (2003) J. Biol. Chem. 278, 23617-23623
19. Chakrabarti, S K, James, J C, and Mirmira, R G. (2002) J. Biol. Chem. 277, 13286-13293
20. Johnson, J. H., Crider, B. P., McCorkle, K., Alford, M. & Unger, R. H. (1990) N Engl J Med 322, 653-9.
21. Antinozzi, P. A., Segall, L., Prentki, M., McGarry, J. D. & Newgard, C. B. (1998) J Biol Chem 273, 16146-54.
22. Jorgensen, M. C., Vestergard Petersen, H., Ericson, J., Madsen, O. D. & Serup, P. (1999) FEBS Lett 461, 287-94.
23. Wang, H., Maechler, P., Ritz-Laser, B., Hagenfeldt, K. A., Ishihara, H., Philippe, J. & Wollheim, C. B. (2001) J Biol Chem 276, 25279-86.
24. Noel, R. J., Antinozzi, P. A., McGarry, J. D. & Newgard, C. B. (1997) J Biol Chem 272, 18621-7.
25. Segall, L., Lameloise, N., Assimacopoulos-Jeannet, F., Roche, E., Corkey, P., Thumelin, S., Corkey, B. E. & Prentki, M. (1999) Am J Physiol 277, E521-8.
26. Wang, H., Maechler, P., Antinozzi, P. A., Herrero, L., Hagenfeldt-Johansson, K. A., Bjorklund, A. & Wollheim, C. B. (2003) J Biol Chem 278, 16622-9.
27. Chen, G., Hohmeier, H. E. & Newgard, C. B. (2001) J Biol Chem 276, 766-72.
28. Tran, V. V., Chen, G., Newgard, C. B. & Hohmeier, H. E. (2003) Diabetes 52, 1423-32.
29. Smukler, S. R., Tang, L., Wheeler, M. B. & Salapatek, A. M. (2002) Diabetes 51, 3450-60.
30. Zhang, S. & Kim, K. H. (1995) FEBS Lett 377, 237-9.
31. Ahren, B. & Havel, P. J. (1999) Am J Physiol 277, R959-66.
32. Merglen, A., Theander, S., Rubi, B., Chaffard, G., Wollheim, C. B. & Maechler, P. (2004) Endocrinology 145, 667-78.
33. Lilla, V., Webb, G., Rickenbach, K., Maturana, A., Steiner, D. F., Halban, P. A. & Irminger, J. C. (2003) Endocrinology 144, 1368-79.
34. Zimmer, Y., Milo-Landesman, D., Svetlanov, A. & Efrat, S. (1999) FEBS Lett 457, 65-70.
35. Kutlu, B., Cardozo, A. K., Darville, M. I., Kruhoffer, M., Magnusson, N., Orntoft, T. & Eizirik, D. L. (2003) Diabetes 52, 2701-19.
36. Rasschaert, J., Liu, D., Kutlu, B., Cardozo, A. K., Kruhoffer, M., T F, O. R. & Eizirik, D. L. (2003) Diabetologia 46, 1641-57.
37. Eizirik, D. L., Kutlu, B., Rasschaert, J., Darville, M. & Cardozo, A. K. (2003) Ann N Y Acad Sci 1005, 55-74.
38. Poitout, V., Olson, L. K. & Robertson, R. P. (1996) Diabetes Metab 22, 7-14.
39. Schwitzgebel, V. M., Scheel, D. W., Conners, J. R., Kalamaras, J., Lee, J. E., Anderson, D. J., Sussel, L., Johnson, J. D. & German, M. S. (2000) Development 127, 3533-42.
40. Wilson, M. E., Scheel, D. & German, M. S. (2003) Mech Dev 120, 65-80.
41. Sussel, L., Kalamaras, J., Hartigan-O'Connor, D. J., Meneses, J. J., Pedersen, R. A., Rubenstein, J. L. & German, M. S. (1998) Development 125, 2213-21.
42. Sander, M., Sussel, L., Conners, J., Scheel, D., Kalamaras, J., Dela Cruz, F., Schwitzgebel, V., Hayes-Jordan, A. & German, M. (2000) Development 127, 5533-40.
43. Jonas, J. C., Sharma, A., Hasenkamp, W., Ilkova, H., Patane, G., Laybutt, R., Bonner-Weir, S. & Weir, G. C. (1999) J Biol Chem 274, 14112-21.

### Example 2

### Nkx6.1 Stimulates Islet Beta Cell Proliferation and Enhances Insulin Secretion

Cell Culture Conditions.

The INS-1-derived cell lines 832/3, 832/13, 834/105, and 833/15 were prepared and cultured as previously described (Chen et al. 2000; Hohmeier et al. 2000). The culture medium was RPMI-1640 with 11.1 mM D-glucose supplemented with 10% fetal bovine serum, 10 mM HEPES, 2 mM L-glutamine, 1 mM sodium pyruvate, and 50 μM β-mercaptoethanol. Primary rat islets were maintained in RPMI-1640 with 8 mM D-glucose supplemented with 10% fetal bovine serum and 100 units/ml of penicillin and 100 μg/ml streptomycin.

Use of Recombinant Adenoviruses for Overexpression and siRNA Mediated Suppression of Gene Expression in INS-1-Derived Cell Lines and Primary Islets.

For gene overexpression studies, recombinant adenoviruses containing the hamster Nkx6.1 cDNA (AdCMV-Nkx6.1), the bacterial β-galactosidase gene (AdCMV-βGAL), myc-tagged human cyclin B1 cDNA downstream of the tetracyclin operator (Ad-t-cyclin B1) and the tetracyclin activator (Ad-tA) were used as previously described (Becker et al. 1994; Jin et al. 1998; Schisler et al. 2005). These viruses were used to treat cell lines at a multiplicity of infection (MOI) ranging from 10-500 for 2 h followed by replacement of virus-containing medium with normal growth medium and further culture for 24-48 h.

For gene suppression studies, adenoviruses expressing small interfering RNAs (siRNAs) specific to rat Nkx6.1 (Ad-siNkx6.1) or a control siRNA with no known gene homology (Ad-siRNAcontrol) were prepared as previously described (Bain et al. 2004; Schisler et al. 2005). The siRNA-expressing adenoviruses were use to treat INS-1-derived cell lines at an MOI of 10-30 for 16 h, followed by replacement of virus-containing medium with fresh culture medium and 80 additional h of tissue culture.

Pancreatic islets were harvested from male Spague-Dawley rats weighing approximately 250 g as previously described using the Liberase R1 enzyme (Roche) (Naber et al. 1980; Milburn et al. 1995). Approximately 200 primary rat islets per condition were cultured in 2 ml of RPMI treated with viruses at a concentration of 5×10⁹ particles/ml medium for 16 h. Virus containing medium was replaced with fresh culture medium and islets were cultured for an additional 80 h after treatment. All viruses were column purified (Vivapure AdenoPACK, Viva Science) and concentrations were determined via OD₂₆₀.

Transfection of INS-1-Derived Cells with siRNA Duplexes.

To use AdCMV-Nkx6.1, AdCMV-βGAL, Ad-t-cyclin B1, and Ad-tA in combination with siRNA-mediated gene silencing in INS-1-derived cells, transfection of siRNA duplexes was used instead of siRNA-expressing adenoviruses to avoid viral toxicity. For siRNA-directed suppression of Nkx6.1 and Cyclin B1 expression, pre-annealed duplexes were obtained from Ambion (ID #195227 and 153133, respectively) and transfected into 832/3 cells via the Amaxa nucleofection system (Amaxa Inc) using 2 μg of duplex per 2×10⁶ cells in T-solution. Cells were seeded at a density of 5×10⁵ cells/ml and cultured for 48 h prior to treating with various adenoviruses as detailed herein. Suppression of the targeted genes was monitored by real-time PCR and immunoblotting.

³H-Thymidine Incorporation and MTS Assays.

Relative change in DNA synthesis in INS-1-derived cell lines was measured similarly to previously described methods (Frodin et al. 1995; Hugl et al. 1998; Dickson et al. 2001). 2.5×10⁵ cells were seeded in 24-well plates and cultured in various conditions. ³H-methyl thymidine (Amersham Biosciences) was added to the medium at a final concentration of 1 μCi/ml during the last 4 h of cell culture. Cells cultured in 24-well plates were placed on ice and washed 3 times with cold serum-free RPMI-1640. DNA was precipitated by adding 1 ml of cold 10% trichloroacetic acid (TCA) followed by a 10 min incubation on ice and two additional precipitation steps with 0.5 ml of 10% TCA and 5 min incubation on ice. The precipitated DNA was solubilized by the addition of 250 μl of 0.3N NaOH and incubated at room temperature for 30-45 min. The amount of ³H-thymdine incorporation into DNA was measured by liquid scintillation counting.

For primary rat islet studies, DNA synthesis rates were measured as described (Cozar-Castellano et al. 2004). ³H-methyl thymidine was added to the medium at a final concentration of 1 μCi/ml during the last 18 h of cell culture to pools of ˜200 islets in various conditions. Groups of 30 islets were picked in triplicate from each condition and washed with 500 μl of cold RPMI-1640. Islets were centrifuged at 300×G for 3 min at 4° C. and the wash was repeated twice. DNA was precipitated with 500 μl of cold 10% TCA and incubated on ice for 10 min. The precipitant was centrifuged at 15,000×G for 3 min at 4° C. after which the precipitation step was repeated. The precipitated DNA was solubilized by the addition of 100 μl of 0.3N NaOH and incubated at room temperature for 30-45 min. The amount of ³H-thymdine incorporated into DNA was measured by liquid scintillation counting, and normalized to total cellular protein measured by the Bradford assay (Bradford 1976).

Cell viability was determined by the MTS assay as previously described (Malich et al. 1997). Briefly, 2.5×10⁵ cells were seeded in 24-well plates and cultured in various conditions. The CellTiter 96 AQueous MTS Assay reagent (Promega) was added directly to the culture medium and incubated for the last hour of culture at 37° C. The reduction of the MTS tetrazolium compound was measured at an absorbance of 490 nm using a SpectraMax 340 plate reader (Molecular Devices). Data were normalized to the % MTS activity per well as well as % MTS activity per mg of protein of control conditions.

BrdU Labeling and Immunohistochemistry in Primary Rat Islets.

For BrdU labeling, a 1/100 dilution of BrdU labeling reagent (Invitrogen) was added to islet culture medium in place of ³H-thymidine for 18 h in various islet conditions as described herein.

Preparation of islets for immunohistochemisty was performed as previously described (Cozar-Castellano et al. 2004). Pools of 200 islets were collected and washed three times with 1 ml of Dulbecco's phosphate buffered saline solution (PBS) with centrifugation at 300×G for 3 min between washes. Islets were fixed in Bouin's solution for 2 h in 1.7 ml microcentrifuge tubes and maintained in 10% neutral-buffered formalin. Prior to embedding in paraffin, islets were centrifuged at 300×G for 3 min and the majority of the formalin was removed by aspiration. 50 μl of Affi-Gel blue bead slurry (Biorad) was added to the islets to allow visualization of the islets during sectioning. Agar was gently added to the mixture in the microcentrifuge tube and allowed to solidify. The agar was removed from the tube and embedded in paraffin.

5 μm serial sections on glass slides were deparaffinized with xylene and rehydrated in a graded series of ethanol. Antigen retrieval was performed by microwaving the slides for 13.5 min in 10 mM sodium citrate buffer with 0.05% Tween-20, pH 6.0. Endogenous peroxidase activity was quenched with 3% H₂O₂ in methanol for 10 min. BrdU immunodetection was carried out using the BrdU detection kit (Invitrogen), which uses a biotinylated mouse anti-BrdU antibody (1 h incubation at room temperature). 3,3′-diaminobenzidine-tetra-hydrochloride (DAB) was used as the HRP substrate for colorimetric detection.

For insulin and glucagon immunostaining, the HistoMouse-MAX kit (Invitrogen) was used with either prediluted guinea pig anti-insulin (Invitrogen) or rabbit anti-glucagon (Invitrogen) primary antibodies incubated for 1 h at room temperature. DAB (brown) or 3-amino-9-ethylcarbazole (AEC, red) were used as

HRP substrates for secondary antibody colorimetric detection. In the case of BrdU and insulin co-staining, detection of anti-insulin antibody was, used with an alkaline phosphatase-conjugated (AP) secondary antibody using Fast Red (Invitrogen) as the AP substrate. Where indicated herein, sections were lightly counterstained with hematoxylin.

Glucose-Stimulated Insulin Secretion.

Pools of 200 islets were treated with various recombinant adenoviruses and washed twice in 2 ml of HEPES balanced salt solution (HBSS) (114 mmol/l NaCl, 4.7 mmol/l KCl, 1.2 mmol/l KH₂PO₄, 1.16 mmol/l MgSO₄, 20 mmol/l HEPES, 2.5 mmol/lCaCl₂, 25.5 mmol/l NaHCO₃, and 0.5% bovine serum albumin [essentially fatty acid free], pH 7.2) containing 2.5 mM glucose. Groups of 30 islets from each condition were picked in triplicate and placed in 2 ml of HBSS with 2.5 mM glucose in 6-well plates for 60 min. Islets were then placed in HBSS with 16.7 mM glucose for an additional 60 min. Medium samples were analyzed by radioimmunoassay (RIA) with the insulin Coat-a-Count kit (Diagnostic Products, Los Angeles, Calif.) (Clark et al. 1997; Hohmeier et al. 1997).

Real-Time PCR Measurements of RNA Levels by RT-PCR.

RNA was harvested from cells or from 20-50 primary rat islets using the RNeasy mini or micro kit (Qiagen), respectively, which included DNase treatment to eliminate genomic contamination. The iScript system (BioRad) was used for first strand synthesis of cDNA using 0.05-0.5 μg of RNA. Real-time PCR reactions were performed using the ABI PRISM® 7000 sequence detection system, software and reagents (An et al. 2004). Primers used with SYBR-Green PCR chemistry were used at final concentrations of 100 nM. Pre-validated primer and probe sets based on Taqman® chemistry (Applied Biosystems) were used as indicated in Table 3. Triplicate reactions from independent RNA samples were carried out in a final volume of 25 μl containing 5 ng of cDNA template. RNA input was calibrated with 18S expression levels and relative mRNA levels were normalized to control conditions as indicated herein.

Confirmation of adenoviral-mediated overexpression of the hamster Nkx6.1 (AdCMV-Nkx6.1) and human cyclin B1 (Ad-t-cyclin B1) transgenes was performed via RT-PCR analysis. 5 ng of cDNA was used in 15 μl reactions (Invitrogen PCR blue supermix) with a final primer concentration of 100 nM. Primer sequences are listed in Table 3. Reactions were activated for 2 min at 95° C. followed by 32 cycles at 95° C. for 15 sec, 53° C. for 15 sec, and 72° C. for 30 sec. 12 μl of each reaction was resolved on a 1.5% agarose gel.

Immunoblot Analysis.

Aliquots of 5.0 μg of nuclear extract protein or 20 μg of whole cell extract protein were resolved on 10% Bis-Tris-HCl buffered (pH=6.4) polyacrylamide gels (Invitrogen) and transferred to polyvinylidene difluoride (PVDF). Membranes were incubated with anti-Nkx6.1 (1:1000, Beta Cell Biology Consortium) or anti-Cyclin B1 (1:1000, Santa Cruz) antibodies overnight at 4° C., or anti-γ-tubulin (1:10,000, Sigma) antibody for 2 h at room temperature. Antibodies were detected using appropriate HRP-linked secondary antibodies and visualized using ECL Advance (Amersham) on a Versadoc 5000 (Biorad).

Microarray Analysis.

RNA was prepared from the 832/13 cell line for microarray analysis. 5×10⁵ cells were seeded into 6 cm plates and cultured overnight. Subsequently cells were treated with either Ad-siRNAcontrol or Ad-siNkx6.1 at an MOI of 20 for 16 h. Total RNA was harvested 72 h later using the RNeasy micro kit (Qiagen), which included DNase treatment to eliminate genomic contamination. Parallel experiments were performed to validate the GSIS phenotype in cells treated with Ad-siNkx6.1 (Schisler et al. 2005) as well as to prepare protein samples.

Duplicate RNA samples (1 μg each) per condition from 3 independent experiments were used for one round of amplification and labeled with Cy5. These samples were hybridized with rat reference RNA labeled with Cy3 to a DNA chip containing the oligonucleotides from the rat operon v1.1 microarray (5600 rat genes, Operon) and the rat 10K OciChip (9715 rat genes, Ocimum) that was scanned on a Gene Pix 5000 scanner. Analysis of the data was performed using the Genespring v7.2 software (Silicon Genetics). Data was normalized using per chip and per spot intensity-dependent LOWESS normalization. Statistical analysis was performed using the software's cross-gene error model using 6 biological replicates per condition. Results of one-way ANOVA (parametric test, variances not assumed equal) were filtered for fold change (greater that 2-fold) and p-values less than 0.05 (Welch t-test)

Chromatin Immunoprecipitation (ChIP) Assays.

ChIP assays were performed as detailed previously (Chakrabarti et al. 2002; Chakrabarti et al. 2003). Antisera used in the immunoprecipitations were either anti-Nkx6.1 antiserum or normal rabbit serum. Each ChIP assay was quantitated in triplicate by real-time PCR for recovery of either the rat cyclin B1 or myoD1 promoters. Three primer sets were used to amplify regions of the cyclin B1 gene. Forward and reverse primers (relative to the transcriptional start site) were, respectively: −1647 bp to −1521 bp 5′-GCTCTGCCATTTATCATCACTGG (SEQ ID NO:30) and 5′-TGACTGCCAAGCAAGGAAGC (SEQ ID NO:31); −1606 bp to −1467 bp 5′-CAGGCTTTCTGTAGCAGTGAGGTG (SEQ ID NO:32) and 5′-GGTTTCTGGTGTGTGTAGCGAAG (SEQ ID NO:33); and −694 bp to −602 bp 5′-TCTCCTGCCCCTACCGTTTTAC (SEQ ID NO:34) and 5′-AACAGATAGCACCCAGACCCTCTC (SEQ ID NO:35). Forward and reverse primers used to amplify the myoD1 gene were, respectively: 5′-CCACTTCGTCCTTGGCTCAAC (SEQ ID NO:36) and 5′-GGGATACCAGGCACAGCATAGG (SEQ ID NO:37).

Statistical Methods.

Statistical significance was determined using a two-tailed Student's t-Test. P-values less than 0.05 were considered significant.

Loss of Nkx6.1 Expression Decreases Beta Cell Proliferation.

To determine if Nkx6.1 expression is involved in the regulation of beta cell proliferation, the effect of siRNA-mediated silencing of Nkx6.1 mRNA on thymidine incorporation into genomic DNA was measured in the Class 3, robustly glucose-responsive INS-1-derived cell line, 833/15 (Chen et al. 2000; Schisler et al. 2005). 833/15 cells were treated with Ad-siRNAcontrol, increasing amounts of Ad-siNkx6.1, or left untreated, followed by assays of proliferation, gene expression, and cell viability.

Treatment with increasing amounts of Ad-siNkx6.1 resulted in an 84-96% decrease in Nkx6.1 mRNA compared to cells treated with Ad-siRNAcontrol, as measured by real-time PCR. Thymidine incorporation decreased in proportion to the dose-dependent suppression of Nkx6.1 expression, with a maximum decrease of 86% (±0.06) compared to control cells. Similar findings were obtained when the same experiment was performed in an independent Class 3 cell line 832/3 (Hohmeier et al. 2000).

Nkx6.1 Overexpression is Sufficient to Increase Beta Cell Proliferation.

Serum deprivation in cell culture systems, including INS-1-derived cell lines, has been shown to increase beta cell death and has been used to study mechanisms of beta cell proliferation (Hugl et al. 1998; Dickson et al. 2001; Ehses et al. 2003; Maestre et al. 2003). Class 1 INS-1-derived cell lines have relatively low levels of Nkx6.1 expression compared to Class 2 and Class 3 cell lines (Schisler et al. 2005). For this reason, the class 1 cell line, 834/105, was used to measure the effect of Nkx6.1 overexpression on beta cell proliferation. 834/105 cells were treated with either AdCMV-βGAL or AdCMV-Nkx6.1, cultured in medium with our without 10% serum and assayed for changes in proliferation.

The withdrawal of serum for 24 h resulted in a 402±9.8% decrease in thymidine incorporation in cells treated with a control adenovirus (AdCMV-βGAL). To investigate the ability of Nkx6.1 expression to compensate for the decrease in proliferation seen with serum deprivation, cells were treated with a recombinant adenovirus containing the Nkx6.1 cDNA (AdCMV-Nkx6.1). Nkx6.1 protein is not detectable in control 834/105 cells, but a clear increase in Nkx6.1 protein is detected in whole cell extracts from cells treated with AdCMV-Nkx6.1. Overexpression was also validated via RT-PCR, showing that the total amount of Nkx6.1 mRNA increases approximately 5-fold with AdCMV-Nkx6.1 treatment. Cells with overexpressed Nkx6.1 experienced only a 176.4% (±7.4%) decrease in thymidine incorporation in response to serum withdrawal, a partial rescue relative to control (AdCMV-βGAL-treated) cells. The effect on Nkx6.1 overexpression to enhance thymidine incorporation was specific to serum-free conditions, as no increases were observed in Nkx6.1 overexpressing cells under normal culture conditions (10% serum).

Nkx6.1 Overexpression Strongly Increases Thymidine Incorporation in Primary Rat Islets.

To begin to investigate the effect of manipulation of Nkx6.1 expression on DNA replication and cell growth in primary rat islets, recombinant adenovirus was used to overexpress Nkx6.1 expression. Primary rat islets were treated with either AdCMV-βGAL or AdCMV-Nkx6.1 and changes in proliferation were measured.

Treatment of primary rat islets with AdCMV-Nkx6.1 increased thymidine incorporation by 7.7±0.7-fold versus cells treated with the AdCMV-βGAL control virus. RT-PCR analysis of RNA isolated from these islets confirmed an increase in Nkx6.1 mRNA of 5-fold in islets treated with AdCMV-Nkx6.1 compared to the AdCMV-βGAL-treated controls. These results demonstrate a potent ability of Nkx6.1 to regulate thymidine incorporation in primary islets, which may reflect effects of the transcription factor on beta cell replication.

Overexpression of Nkx6.1 in Islets Increases BrdU Immunoreactivity in Islet Beta Cells.

The increase in thymidine incorporation in Ad-Nkx6.1-treated rat islets could be explained by stimulation of beta cell proliferation, but could also be due to replication of other islet cell types or non-islet fibroblastic cells. Also, whereas increases in thymidine incorporation are generally indicative of activation of cell replication, more direct measures are required to validate this premise. To investigate these issues further, uptake of the nucleotide analog 5′-bromo-2′-deoxyuridine (BrdU) into rat islets was measured by immunohistochemistry. BrdU was detected in representative sections (5 μm) of primary rat islets treated with either AdCMV-βGAL or AdCMV-Nkx6.1 at either 20× or 40× magnification.

Islets treated with AdCMV-Nkx6.1 had a striking increase in the number of BrdU positive cells. Nearly all sections from islets overexpressing Nkx6.1 had multiple BrdU positive cells (range of 0-9 BrdU positive cells/section, average of 154 sections=3.12) with many pairs of adjacent stained cells that appeared to have recently divided, whereas sections from untreated or AdCMV-βGAL-treated control islets usually had no BrdU positive cells (range of 0-4 BrdU positive cells/section, average of 213 sections=0.39). The fold increase in the number of BrdU positive cells per section in islets treated with AdCMV-Nkx6.1 versus AdCMV-βGAL was 8.1±1.3-fold. These data are well correlated with the large increase in thymidine incorporation described above and support a robust increase in the number of islet cells undergoing cell division in response to Nkx6.1 overexpression.

To determine if the BrdU positive cells are beta cells, sections were co-stained with antibodies specific for BrdU and insulin. This analysis revealed that 79% of the BrdU positive cells in AdCMV-Nkx6.1-treated islets co-stained with insulin, showing that the majority of replicating cells were beta cells. In contrast, the rare BrdU positive cells in control islets were comprised of a roughly equal number of insulin-positive and insulin-negative cell types. Taken together, the major effect of Nkx6.1 overexpression is to increase islet beta cell proliferation, consistent with the beta cell specific expression of the transcription factor in islet development.

Overexpression of Nkx6.1 increases Islet Cell Number. To determine if the Nkx6.1-induced increases in ³H thymidine and BrdU incorporation noted above actually equate to increased β-cell replication, two randomly aliquoted pools of approximately 300 rat islets were treated with AdCMV-Nkx6.1 or AdCMV-βGAL for 18 hours, and then cultured for an additional 50 hours. The two pools were split into 10 groups of 30 islets, prior to dispersal and counting of the number of cells. The AdCMV-Nkx6.1 treated islets contained 881 more cells than the AdCMV-βGAL-treated islets, an increase of 33±8%.

siRNA-Mediated Suppression of Nkx6.1 Expression in Primary Rat Islets Suppresses Thymidine Incorporation and Causes Dramatic Changes in Islet Cell Morphology.

To study the effects of Nkx6.1 suppression, rat islets were treated with a virus containing an siRNA specific to Nkx6.1 (Ad-siNkx6.1) or a control siRNA (Ad-siRNAcontrol) (Schisler et al., 2005). Treatment of islets with Ad-siNkx6.1 reduced the levels of Nkx6.1 mRNA by 56%±10% compared to islets treated with Ad-siRNAcontrol, as measured by real-time PCR. Strikingly, islets with reduced Nkx6.1 expression had a 58±8% decrease in thymidine uptake relative to islets treated with Ad-siRNAcontrol.

Despite the decrease in thymidine incorporation, islets with reduced Nkx6.1 expression were markedly enlarged relative to control islets. To better understand this phenomenon, islet sections were stained with hematoxylin and eosin. This analysis revealed that the larger, Ad-siNkx6.1-treated islets have a disorganized core of cells. In addition, there is an apparent increase in the size of individual cells in Ad-siNkx6.1-treated islets compared to Ad-siRNAcontrol-treated islets, suggesting that suppression of Nkx6.1 expression in mature beta-cells results in islet hypertrophy, possibly secondary to interruption of the cell cycle. Immunohistochemical analysis of islets treated with Ad-siNkx6.1 revealed that the core cells of these islets do not express insulin, whereas control islets exhibit prominent insulin staining in the islet core. Peripheral glucagon staining was similar in Ad-siRNAcontrol versus Ad-siNkx6.1 treated islets. Additionally, the insulin-negative core of Ad-siNkx6.1-treated islets are also negative for glucagon expression. Taken together, these results suggest that Nkx6.1 plays important roles in maintenance of normal islet cell architecture and islet cell size, in addition to its effects on islet cell replication.

Microarray Analysis—siRNA-Nkx6.1 in 832/13 Cells

Microarray analysis was carried out on 832/13 cells treated with Ad-siNkx6.1 or Ad-siRNAcontrol. Duplicate RNA samples per condition from three independent experiments were collected and hybridized to a rat oligonucleotide array containing approximately 10,000 rat genes. While originally described as a suppressor of gene expression, a recent study has indicated that Nkx6.1 may also serve as an enhancer, as Nkx6.1 stimulates its own expression (Iype et al. 2004). The present microarray analysis revealed that suppression of Nkx6.1 resulted in up-regulated expression of 76 genes by two-fold or more, consistent with its role as a transcriptional repressor, but also results in suppression of an additional 38 genes by 50% or more, demonstrating a broader transcriptional activator role for this factor than previously realized. Changes in expression in response to Nkx6.1 suppression for a number of selected transcripts as well as the confirmation that Nkx6.1 was effectively suppressed (by 66%) in these studies was confirmed via real-time PCR. The complete list of genes with the relative fold change and brief gene description is provided in Table 6.

Microarray Analysis Reveals that Nkx6.1 Regulates a Broad Array of Cell Cycle Regulatory Genes.

To better understand the novel roles of Nkx6.1 to regulate islet cell replication, microarray analysis was performed on primary rat islets treated with AdCMV-Nkx6.1 or AdCMV-βGAL. Islets were treated with AdCMV-βGAL or AdCMV-Nkx6.1 recombinant adenoviruses for 18 h, and then maintained in culture for an additional 50 h. Subsequently, islets were isolated for mRNA isolation and expression of the indicated genes was analyzed by quantitative real-time PCR. Duplicate RNA samples per condition from three independent experiments were collected and hybridized to a rat oligonucleotide array containing approximately 10,000 rat genes as described herein. The microarray analysis reveals that overexpression of Nkx6.1 results in suppression or downregulation of 168 genes by 50% or more, consistent with its role as a transcriptional repressor, but also results in upregulated expression of 146 genes by two-fold or more, demonstrating a broader transcriptional activator role for this factor than previously realized. Moreover, Nkx6.1 overexpression results in large increases in expression of a number of cell cycle regulatory genes, including cyclins A2, B1, B2, and E1, Cdk 1 and 2, Cdc6 and 25a, and PTTG1 (Table 5). These genes are involved in all phases of the cell cycle, from G1 to M. To confirm the findings of the microarray analysis, real time PCR was used to measure mRNA levels for the various cell cycle control genes in AdCMV-Nkx6.1-treated islets versus AdCMV-βGAL-treated control islets. Importantly, all of the cell cycle regulatory genes that were found to be upregulated by microarray were confirmed to be upregulated by RT-PCR analysis. These experiments reveal a striking and heretofore unsuspected effect of Nkx6.1 to upregulate a broad array of cell cycle regulatory genes.

Chromatin Immunoprecipitation Reveals that Nkx6.1 Interacts Directly with the Cyclin B1 Promoter.

The data provided herein show that Nkx6.1 contributes to the regulation of a broad array of cell cycle regulatory genes, including cyclins A2, B1, and E1. To investigate the possibility that Nkx6.1 exerts these effects via direct interaction with the cyclin genes, the sequences of the rat cyclin A2, B1, and E1 promoters (e.g., GenBank Accession number F046121) were analyzed for sequences homologous to a recently described Nkx6.1 enhancer sequence within the Nkx6.1 gene promoter (Iype et al. 2004). As shown in Table 4, all three genes contain multiple sequences with the minimum—ATTT—core element necessary for Nkx6.1-mediated activation. Moreover, within each of the 5′ flanking regions of the cyclin A2, B1, and E1 genes, multiple—ATTT—core sequences were found to be flanked by sequence high in GC content, another functional characteristic shared with the Nkx6.1 enhancer sequence (Iype et al. 2004). Conversely, other targets (−518 to −502 and −119 to −103) are flanked by sequence relatively low in GC content. To determine which sites might interact with Nkx6.1, PCR primers were designed to target different regions of the cyclin A2, B1, and E2 promoters. The resulting ChIP analyses show a clear enrichment of a PCR product immunoprecipitated with Nkx6.1 antisera compared to control sera with primer set A and B (spanning the region from −1647 to −1467), whereas no enrichment can be seen with primer set C (located in the region from −694 to −602). Real-time PCR analysis controls for PCR efficiency and allows comparisons to be made across multiple PCR products (Chakrabarti et al. 2002; Chakrabarti et al. 2003). Real-time PCR analysis confirms a 5-fold enrichment in DNA only with primer sets A and B and no enrichment with primer set C.

Effects of Cyclin B1 Silencing and Overexpression on Beta Cell Proliferation.

The foregoing results imply that Nkx6.1 may regulate beta cell proliferation via regulation of cyclins and other cell cycle regulatory genes. Experiments were conducted to test the ability of cyclin B1 overexpression to rescue cells with decreased Nkx6.1 expression. To this end, rat islets were treated with Ad-siRNAcontrol or Ad-siNkx6.1 in the presence of Ad-tA or Ad-tA plus Ad-t-cyclinB1.

Treatment of islets with Ad-siNkx6.1 decreased thymidine incorporation by 47% (±3%), whereas overexpression of cyclin B1 was able to reverse the Ad-siNkx6.1-mediated decrease, bringing thymidine incorporation back to control levels. Human cyclin B1 can be detected only in RNA samples from islets treated with both Ad-tA and Ad-t-cyclin B1. A clear increase in total cyclin B1 levels can be seen with a primer set that recognizes both rat and human cyclin B1. These data suggest that cyclin B1 plays a major role in mediating the effect of Nkx6.1 to suppress islet proliferation, but that other cell cycle regulatory factors controlled by Nkx6.1 are required to achieve the strong effect of Nkx6.1 overexpression on islet growth.

Overexpression of Nkx6.1 does not Impair GSIS or Alter Expression of Key Beta Cell Genes.

In almost all cases reported to date, increases in beta cell replication caused by oncogene expression or growth factor and cell matrix manipulations are accompanied by loss of differentiated function, particularly decreases in insulin content and glucose-stimulated insulin secretion (Welsh et al. 1988; Beattie et al. 1991; Beattie et al. 1996; Yuan et al. 1996; Beattie et al. 1997; Laybutt et al. 2002; Hohmeier and Newgard, Nature Biotechnology). To determine if the growth promoting effects of Nkx6.1 overexpression are linked to similar functional derangements, GSIS and insulin content were measured in rat islets treated with AdCMV-Nkx6.1 or AdCMV-βGAL used in the ³H-thymidine incorporation assay described herein. Nkx6.1 overexpression was confirmed via RT-PCR as well as real-time PCR.

Overexpression of Nkx6.1 caused no significant change in basal insulin secretion (measured at 2.5 mM glucose) compared to islets overexpressing 3-galactosidase. However, Nkx6.1 overexpression caused a 46% increase in insulin secretion at a stimulatory glucose concentration (16.7 mM) (1398.5±135.3 vs 958.8±39.3 μU/mg protein, p=0.004). Therefore, Nkx6.1 overexpression certainly does not impair beta cell function as measured by GSIS, and actually causes an increase in insulin secretion at stimulatory glucose concentrations.

It has been previously suggested that Nkx6.1 suppresses insulin expression (Mirmira et al. 2000; Iype et al. 2004; Taylor et al. 2005). These conclusions were based on the ability of Nkx6.1 to suppress a reporter gene with a portion of the insulin promoter cloned upstream of a minimal promoter. In addition, chromatin immunoprecipitation (ChIP) analysis in beta cell lines has shown that Nkx6.1 interacts directly with the insulin promoter in vivo. Using primers that recognize rat insulin 2 gene products (Iype et al. 2005), the effects of Nkx6.1 overexpression on insulin gene transcription were measured. Neither mature or pre-mRNA species of insulin transcript were altered; the latter has been shown to reflect acute changes in insulin transcription due to its short half-life (8 min) (Iype et al. 2005). In addition, Nkx6.1 overexpression caused no significant changes in levels of Pdx1, GLUT2, or glucokinase mRNAs. Taken together, these data indicate that overexpression of Nkx6.1 in primary rat islets does not impair expression of insulin or other key genes of the differentiated beta cell.

In summary, the current study shows that Nkx6.1 regulates beta cell proliferation, as supported by several specific findings. First, siRNA-mediated silencing of Nkx6.1 expression results in a dramatic decrease in proliferation in both beta cell lines and primary rat islets, as measured by thymidine incorporation into genomic DNA. Second, overexpression of Nkx6.1 restores the proliferative capacity of serum deprived INS-derived cells, a condition that normally causes cessation of cell growth and beta cell death. Third, overexpression of Nkx6.1 in normal rat islets increases thymidine incorporation by 7-fold. Moreover, immunohistochemical measurements of BrdU incorporation confirm the increase in primary cell replication, and demonstrate that most if not all of the proliferating cells are beta cells. Nkx6.1 expression also clearly increased the total numbers of cells in primary islet preparations.

Importantly, the Nkx6.1-mediated increase in beta cell proliferation does not impair beta cell function, as evaluated by studies of glucose-stimulated insulin secretion and measurement of expression levels (mRNA) of several important beta cell genes. In fact, overexpression of Nkx6.1 causes insulin secretion to be significantly increased at stimulatory glucose levels, with no change in insulin content, suggesting a specific enhancement of the secretory response. This is consistent with previous work, which demonstrated that siRNA-mediated suppression of Nkx6.1 expression causes pronounced impairment of GSIS in both primary islets and beta cell lines, with this effect also being independent of changes in insulin mRNA or content (Schisler et al. 2005).

### References for Example 2

An, J., D. M. Muoio, M. Shiota, Y. Fujimoto, G. W. Cline, G. I. Shulman, T. R. Koves, R. Stevens, D. Millington and C. B. Newgard (2004). “Hepatic expression of malonyl-CoA decarboxylase reverses muscle, liver and whole-animal insulin resistance.” Nat Med 10(3): 268-74.
Bain, J. R., J. C. Schisler, K. Takeuchi, C. B. Newgard and T. C. Becker (2004). “An adenovirus vector for efficient RNA interference-mediated suppression of target genes in insulinoma cells and pancreatic islets of langerhans.” Diabetes 53(9): 2190-4.
Beattie, G. M., V. Cirulli, A. D. Lopez and A. Hayek (1997). “Ex vivo expansion of human pancreatic endocrine cells.” J Clin Endocrinol Metab 82(6): 1852-6.
Beattie, G. M., D. A. Lappi, A. Baird and A. Hayek (1991). “Functional impact of attachment and purification in the short term culture of human pancreatic islets.” J Clin Endocrinol Metab 73(1): 93-8.
Beattie, G. M., A. M. Montgomery, A. D. Lopez, E. Hao, B. Perez, M. L. Just, J. R. Lakey, M. E. Hart and A. Hayek (2002). “A novel approach to increase human islet cell mass while preserving beta-cell function.” Diabetes 51(12): 3435-9.
Beattie, G. M., J. S. Rubin, M. I. Mally, T. Otonkoski and A. Hayek (1996). “Regulation of proliferation and differentiation of human fetal pancreatic islet cells by extracellular matrix, hepatocyte growth factor, and cell-cell contact.” Diabetes 45(9): 1223-8.
Becker, T. C., R. J. Noel, W. S. Coats, A. M. Gomez-Foix, T. Alam, R. D. Gerard and C. B. Newgard (1994). “Use of recombinant adenovirus for metabolic engineering of mammalian cells.” Methods Cell Biol 43 Pt A: 161-89.
Bernal-Mizrachi, E., W. Wen, S. Stahlhut, C. M. Welling and M. A. Permutt (2001). “Islet beta cell expression of constitutively active Akt1/PKB alpha induces striking hypertrophy, hyperplasia, and hyperinsulinemia.” J Clin Invest 108(11): 1631-8.
Bonner-Weir, S. (2001). “beta-cell turnover: its assessment and implications.” Diabetes 50 Suppl 1: S20-4.
Bradford, M. M. (1976). “A rapid and sensitive method for the quantitation of microgram quantities of protein utilizing the principle of protein-dye binding.” Anal Biochem 72: 248-54.
Chakrabarti, S. K., J. Francis, S. M. Ziesmann, J. C. Garmey and R. G. Mirmira (2003). “Covalent histone modifications underlie the developmental regulation of insulin gene transcription in pancreatic beta cells.” J Biol Chem 278(26): 23617-23.
Chakrabarti, S. K., J. C. James and R. G. Mirmira (2002). “Quantitative assessment of gene targeting in vitro and in vivo by the pancreatic transcription factor, Pdx1. Importance of chromatin structure in directing promoter binding.” J Biol Chem 277(15): 13286-93.
Chakrabarti, S. K. and R. G. Mirmira (2003). “Transcription factors direct the development and function of pancreatic beta cells.” Trends Endocrinol Metab 14(2): 78-84.
Chen, G., H. E. Hohmeier, R. Gasa, V. V. Tran and C. B. Newgard (2000). “Selection of insulinoma cell lines with resistance to interleukin-1 beta- and gamma-interferon-induced cytotoxicity.” Diabetes 49(4): 562-70.
Clark, S. A., C. Quaade, H. Constandy, P. Hansen, P. Halban, S. Ferber, C. B. Newgard and K. Normington (1997). “Novel insulinoma cell lines produced by iterative engineering of GLUT2, glucokinase, and human insulin expression.” Diabetes 46(6): 958-67.
Cozar-Castellano, I., K. K. Takane, R. Bottino, A. N. Balamurugan and A. F. Stewart (2004). “Induction of beta-cell proliferation and retinoblastoma protein phosphorylation in rat and human islets using adenovirus-mediated transfer of cyclin-dependent kinase-4 and cyclin D1.” Diabetes 53(1): 149-59.
de la Tour, D., T. Halvorsen, C. Demeterco, B. Tyrberg, P. Itkin-Ansari, M. Loy, S. J. Yoo, E. Hao, S. Bossie and F. Levine (2001). “Beta-cell differentiation from a human pancreatic cell line in vitro and in vivo.” Mol Endocrinol 15(3): 476-83.
Del Bene, F. and J. Wittbrodt (2005). “Cell cycle control by homeobox genes in development and disease.” Semin Cell Dev Biol 16(3): 449-60.
Dickson, L. M., M. K. Lingohr, J. McCuaig, S. R. Hugl, L. Snow, B. B. Kahn, M. G. Myers, Jr. and C. J. Rhodes (2001). “Differential Activation of Protein Kinase B and p70S6K by Glucose and Insulin-like Growth Factor 1 in Pancreatic beta-Cells (INS-1).” J. Biol. Chem. 276(24): 21110-21120.
Ehses, J. A., V. R. Casilla, T. Doty, J. A. Pospisilik, K. D. Winter, H.-U. Demuth, R. A. Pederson and C. H. S. McIntosh (2003). “Glucose-Dependent Insulinotropic Polypeptide Promotes {beta}-(INS-1) Cell Survival via Cyclic Adenosine Monophosphate-Mediated Caspase-3 Inhibition and Regulation of p38 Mitogen-Activated Protein Kinase.” Endocrinology 144(10): 4433-4445.
Frodin, M., N. Sekine, E. Roche, C. Filloux, M. Prentki, C. B. Wollheim and E. Van Obberghen (1995). “Glucose, other secretagogues, and nerve growth factor stimulate mitogen-activated protein kinase in the insulin-secreting beta-cell line, INS-1.” J Biol Chem 270(14): 7882-9.
Garcia-Ocana, A., K. K. Takane, V. T. Reddy, J. C. Lopez-Talayera, R. C. Vasavada and A. F. Stewart (2003). “Adenovirus-mediated hepatocyte growth factor expression in mouse islets improves pancreatic islet transplant performance and reduces beta cell death.” J Biol Chem 278(1): 343-51.
Garcia-Ocana, A., K. K. Takane, M. A. Syed, W. M. Philbrick, R. C. Vasavada and A. F. Stewart (2000). “Hepatocyte growth factor overexpression in the islet of transgenic mice increases beta cell proliferation, enhances islet mass, and induces mild hypoglycemia.” J Biol Chem 275(2): 1226-32.
Garcia-Ocana, A., R. C. Vasavada, A. Cebrian, V. Reddy, K. K. Takane, J. C. Lopez-Talayera and A. F. Stewart (2001). “Transgenic overexpression of hepatocyte growth factor in the beta-cell markedly improves islet function and islet transplant outcomes in mice.” Diabetes 50(12): 2752-62.
Georgia, S. and A. Bhushan (2004). “Beta cell replication is the primary mechanism for maintaining postnatal beta cell mass.” J Clin Invest 114(7): 963-8.
Habener, J. F., D. M. Kemp and M. K. Thomas (2005). “Minireview: transcriptional regulation in pancreatic development.” Endocrinology 146(3): 1025-34.
Henseleit, K. D., S. B. Nelson, K. Kuhlbrodt, J. C. Hennings, J. Ericson and M. Sander (2005). “NKX6 transcription factor activity is required for alpha- and beta-cell development in the pancreas.” Development 132(13): 3139-49.
Hino, S., T. Yamaoka, Y. Yamashita, T. Yamada, J. Hata and M. Itakura (2004). “In vivo proliferation of differentiated pancreatic islet beta cells in transgenic mice expressing mutated cyclin-dependent kinase 4.” Diabetologia 47(10): 1819-30.
Hohmeier, H. E., H. BeltrandelRio, S. A. Clark, R. Henkel-Rieger, K. Normington and C. B. Newgard (1997). “Regulation of insulin secretion from novel engineered insulinoma cell lines.” Diabetes 46(6): 968-77.
Hohmeier, H. E., H. Mulder, G. Chen, R. Henkel-Rieger, M. Prentki and C. B. Newgard (2000). “Isolation of INS-1-derived cell lines with robust ATP-sensitive K+ channel-dependent and -independent glucose-stimulated insulin secretion.” Diabetes 49(3): 424-30.
Hohmeier, H. E. and C. B. Newgard (2005). “Islets for all?” Nat Biotechnol 23(10): 1231-2.
Hugl, S. R., M. F. White and C. J. Rhodes (1998). “Insulin-like Growth Factor I (IGF-I)-stimulated Pancreatic beta-Cell Growth Is Glucose-dependent. SYNERGISTIC ACTIVATION OF INSULIN RECEPTOR SUBSTRATE-MEDIATED SIGNAL TRANSDUCTION PATHWAYS BY GLUCOSE AND IGF-I IN INS-1 CELLS.” J. Biol. Chem. 273(28): 17771-17779.
Iype, T., J. Francis, J. C. Garmey, J. C. Schisler, R. Nesher, G. C. Weir, T. C. Becker, C. B. Newgard, S. C. Griffen and R. G. Mirmira (2005). “Mechanism of insulin gene regulation by the pancreatic transcription factor Pdx-1: application of pre-mRNA analysis and chromatin immunoprecipitation to assess formation of functional transcriptional complexes.” J Biol Chem 280(17): 16798-807.
Iype, T., D. G. Taylor, S. M. Ziesmann, J. C. Garmey, H. Watada and R. G. Mirmira (2004). “The Transcriptional Repressor Nkx6.1 Also Functions as a Deoxyribonucleic Acid Context-Dependent Transcriptional Activator during Pancreatic {beta}-Cell Differentiation: Evidence for Feedback Activation of the nkx6.1 Gene by Nkx6.1.” Mol Endocrinol 18(6): 1363-1375.
Jackman, M., C. Lindon, E. A. Nigg and J. Pines (2003). “Active cyclin B1-Cdk1 first appears on centrosomes in prophase.” Nat Cell Biol 5(2): 143-8.
Jin, P., S. Hardy and D. O. Morgan (1998). “Nuclear Localization of Cyclin B1 Controls Mitotic Entry After DNA Damage.” J. Cell Biol. 141(4): 875-885.
Kushner, J. A., M. A. Ciemerych, E. Sicinska, L. M. Wartschow, M. Teta, S. Y. Long, P. Sicinski and M. F. White (2005). “Cyclins D2 and D1 are essential for postnatal pancreatic beta-cell growth.” Mol Cell Biol 25(9): 3752-62.
Laybutt, D. R., G. C. Weir, H. Kaneto, J. Lebet, R. D. Palmiter, A. Sharma and S. Bonner-Weir (2002). “Overexpression of c-Myc in beta-cells of transgenic mice causes proliferation and apoptosis, downregulation of insulin gene expression, and diabetes.” Diabetes 51(6): 1793-804.
Lebovitz, H. E. (2004). Chapter 76: Insulin Secretagogues: Sulfonylureas, Meglitinides, and Phenylalanine Derivatives. Diabetes Mellitus. D. LeRoith, Taylor, S. I., Olefsky, J. M. Philadelphia, Lippincott Williams & Wilkins: 1107-1138.
Maestre, I., J. Jordan, S. Calvo, J. A. Reig, V. Cena, B. Soria, M. Prentki and E. Roche (2003). “Mitochondrial Dysfunction Is Involved in Apoptosis Induced by Serum Withdrawal and Fatty Acids in the {beta}-Cell Line Ins-1.” Endocrinology 144(1): 335-345.
Magne, S., S. Caron, M. Charon, M. C. Rouyez and I. Dusanter-Fourt (2003). “STAT5 and Oct-1 form a stable complex that modulates cyclin D1 expression.” Mol Cell Biol 23(24): 8934-45.
Malich, G., B. Markovic and C. Winder (1997). “The sensitivity and specificity of the MTS tetrazolium assay for detecting the in vitro cytotoxicity of 20 chemicals using human cell lines.” Toxicology 124(3): 179-92.
Marzo, N., C. Mora, M. E. Fabregat, J. Martin, E. F. Usac, C. Franco, M. Barbacid and R. Gomis (2004). “Pancreatic islets from cyclin-dependent kinase 4/R24C (Cdk4) knockin mice have significantly increased beta cell mass and are physiologically functional, indicating that Cdk4 is a potential target for pancreatic beta cell mass regeneration in Type 1 diabetes.” Diabetologia 47(4): 686-94.
Melloul, D. (2004). “Transcription factors in islet development and physiology: role of PDX-1 in beta-cell function.” Ann N Y Acad Sci 1014: 28-37.
Milburn, J. L., Jr., H. Hirose, Y. H. Lee, Y. Nagasawa, A. Ogawa, M. Ohneda, H. BeltrandelRio, C. B. Newgard, J. H. Johnson and R. H. Unger (1995). “Pancreatic beta-cells in obesity. Evidence for induction of functional, morphologic, and metabolic abnormalities by increased long chain fatty acids.” J Biol Chem 270(3): 1295-9.
Mirmira, R. G., H. Watada and M. S. German (2000). “Beta-cell differentiation factor Nkx6.1 contains distinct DNA binding interference and transcriptional repression domains.” J Biol Chem 275(19): 14743-51.
Naber, S. P., J. M. McDonald, L. Jarett, M. L. McDaniel, C. W. Ludvigsen and P. E. Lacy (1980). “Preliminary characterization of calcium binding in islet-cell plasma membranes.” Diabetologia 19(5): 439-44.
Nurse, P. (1990). “Universal control mechanism regulating onset of M-phase.” Nature 344(6266): 503-8.
Ozawa, H., S. Ashizawa, M. Naito, M. Yanagihara, N. Ohnishi, T. Maeda, Y. Matsuda, Y. Jo, H. Higashi, A. Kakita and M. Hatakeyama (2004). “Paired-like homeodomain protein ESXR1 possesses a cleavable C-terminal region that inhibits cyclin degradation.” Oncogene 23(39): 6590-602.
Pines, J. and C. L. Rieder (2001). “Re-staging mitosis: a contemporary view of mitotic progression.” Nat Cell Biol 3(1): E3-6.
Qiu, M., K. Shimamura, L. Sussel, S. Chen and J. L. Rubenstein (1998). “Control of anteroposterior and dorsoventral domains of Nkx-6.1 gene expression relative to other Nkx genes during vertebrate CNS development.” Mech Dev 72(1-2): 77-88.
Rane, S. G., P. Dubus, R. V. Mettus, E. J. Galbreath, G. Boden, E. P. Reddy and M. Barbacid (1999). “Loss of Cdk4 expression causes insulin-deficient diabetes and Cdk4 activation results in beta-islet cell hyperplasia.” Nat Genet 22(1): 44-52.
Rudnick, A., T. Y. Ling, H. Odagiri, W. J. Rutter and M. S. German (1994). “Pancreatic beta cells express a diverse set of homeobox genes.” Proc Natl Acad Sci USA 91(25): 12203-7.
Sander, M., L. Sussel, J. Conners, D. Scheel, J. Kalamaras, F. Dela Cruz, V. Schwitzgebel, A. Hayes-Jordan and M. German (2000). “Homeobox gene Nkx6.1 lies downstream of Nkx2.2 in the major pathway of beta-cell formation in the pancreas.” Development 127(24): 5533-40.
Schisler, J. C., P. B. Jensen, D. G. Taylor, T. C. Becker, F. K. Knop, S. Takekawa, M. German, G. C. Weir, D. Lu, R. G. Mirmira and C. B. Newgard (2005). “The Nkx6.1 homeodomain transcription factor suppresses glucagon expression and regulates glucose-stimulated insulin secretion in islet beta cells.” Proc Natl Acad Sci USA 102(20): 7297-302.
Taylor, D. G., D. Babu and R. G. Mirmira (2005). “The C-terminal domain of the beta cell homeodomain factor Nkx6.1 enhances sequence-selective DNA binding at the insulin promoter.” Biochemistry 44(33): 11269-78.
Topp, B., K. Promislow, G. deVries, R. M. Miura and D. T. Finegood (2000). “A model of beta-cell mass, insulin, and glucose kinetics: pathways to diabetes.” J Theor Biol 206(4): 605-19.
Tuttle, R. L., N. S. Gill, W. Pugh, J. P. Lee, B. Koeberlein, E. E. Furth, K. S. Polonsky, A. Naji and M. J. Birnbaum (2001). “Regulation of pancreatic beta-cell growth and survival by the serine/threonine protein kinase Akt1/PKBalpha.” Nat Med 7(10): 1133-7.
Weir, G. C. and S. Bonner-Weir (2004). “Five Stages of Evolving Beta-Cell Dysfunction During Progression to Diabetes.” Diabetes 53(3): S16-21.
Welsh, M., N. Welsh, T. Nilsson, P. Arkhammar, R. B. Pepinsky, D. F. Steiner and P. O. Berggren (1988). “Stimulation of pancreatic islet beta-cell replication by oncogenes.” Proc Natl Acad Sci USA 85(1): 116-20.
Yuan, S., L. Rosenberg, S. Paraskevas, D. Agapitos and W. P. Duguid (1996). “Transdifferentiation of human islets to pancreatic ductal cells in collagen matrix culture.” Differentiation 61(1): 67-75.

### Example 3

### Efficient Gene Delivery to Pancreatic Cells with Ultrasonic Microbubble Destruction Technology

Summary of Study.

This study describes a novel method of gene delivery to pancreatic islets of adult, living animals by ultrasound-targeted microbubble destruction (UTMD) technology. The technique involves incorporation of plasmids into the phospholipid shell of gas-filled microbubbles, which are then infused into rats and destroyed within the pancreatic microcirculation using ultrasound. Specific delivery of genes to islet beta-cells by UTMD was achieved by use of a plasmid containing a rat insulin promoter (RIP), and reporter gene expression was regulated appropriately by glucose in animals that received a RIP-luciferase plasmid. To demonstrate biological efficacy, UTMD was used to deliver RIP-human insulin and RIP-hexokinase I plasmids to islets of adult rats. Delivery of the former plasmid resulted in clear increases in circulating human C-peptide and blood glucose levels, whereas delivery of the latter plasmid resulted in a clear increase in hexokinase I protein expression in islets, increased insulin levels in blood, and decreased circulating glucose levels. These studies demonstrate that UTMD allows relatively non-invasive delivery of genes to pancreatic islets with an efficiency sufficient to modulate beta-cell function in adult animals.

Rat UTMD Protocol.

Sprague-Dawley rats (250-350 g) were anesthetized with intraperitoneal ketamine (100 mg/kg) and xylazine (5 mg/kg). A polyethylene tube (PE 50, Becton Dickinson, Md.) was inserted into the right internal jugular vein by cutdown. The anterior abdomen was shaved and an S3 probe (Sonos 5500, Philips Ultrasound, Andover, Mass.) placed to image the left kidney and spleen, which are easily identified. The pancreas lies between them, so the probe was adjusted to target the pancreas and clamped in place. Plasmid DNA containing the reporter genes LacZ, DsRed, or luciferase, or the hexokinase-1 gene under the regulation of either CMV or RIP promoters were incorporated within the phospholipid shell of perfluoropropane gas-filled microbubbles. One ml of microbubble solution was infused at a constant rate of 3 ml/h for 20 minutes using an infusion pump. Ultrasound was directed at the pancreas to destroy these microbubbles within the pancreatic microcirculation; microbubble infusion without ultrasound was also used as a control. Throughout the duration of the infusion, microbubble destruction was achieved using ultraharmonic mode (transmit 1.3 MHz/receive 3.6 MHz) with a mechanical index of 1.2-1.4 and a depth of 4 cm. The ultrasound pulses were ECG-triggered (at 80 ms after the peak of the R wave) to deliver a burst of 4 frames of ultrasound every 4 cardiac cycles. These settings have previously been shown to be the optimal ultrasound parameters for gene delivery using UTMD¹¹. At the end of each experiment the jugular vein was tied off and the skin closed. All rats were monitored after the experiment for normal behavior. Rats were sacrificed 4 days later and the pancreas was removed for further analysis.

Manufacture of Plasmid-Containing Lipid-Stabilized Microbubbles.

Lipid-stabilized microbubbles were prepared as previously described¹¹. A stock solution of DPPC (1,2-dipalmitoyl-sn-glycero-3-phosphatidylcholine, Sigma, St. Louis, Mo.) 250 mg; DPPE (1,2-dipalmitoyl-sn-glycero-3-phosphatidylethanolamine, Sigma, St. Louis, Mo.) 50 mg; and 10% glucose 1000 mg was mixed with PBS to a final volume of 10 ml. This was boiled in water until all powder was fully dissolved. Then, 2 mg of plasmid DNA was mixed with 0.5 ml ethyl alcohol and centrifuged at 14,000 rpm for 5 minutes. The supernate was removed and the DNA pellet was placed in an incubator at 37° C. for 5 minutes to remove any remaining ethyl alcohol. The DNA was then added to 50 μl of lipofectamine 2000 (1 mg/ml) and mixed for 20 minutes until fully dissolved. This DNA/lipofectamine was then added to 250 μl liposome stock solution, 5 μl of 10% albumin, and 50 μl of glycerol (10 mg/ml) in 1.5 ml vials, mixed well and placed on ice. The headspace of the vial was then filled with C3F8 gas (Air Products, Allentown, Pa.), capped and shaken for 30 minutes at 4° C. The lipid-stabilized microbubbles appear as a milky white suspension floating on the top of a layer of liquid containing unincorporated plasmid DNA. The subnatant was discarded and the microbubbles washed three times with PBS to removed unincorporated plasmid DNA. The mean diameter and concentration of the microbubbles in the upper layer were measured by a particle counter (Beckman Coulter Multisizer III). The mean diameter and concentration of the microbubbles were 1.9±0.2 μm and 5.2±0.3×10⁹ bubbles/ml, respectively. The amount of plasmid carried by the microbubbles was 250±10 μg/ml.

Plasmid Constructs.

Rat genomic DNA was extracted from rat peripheral blood with a QIAamp Blood kit (Qiagen Inc, Valencia, Calif.) according to the manufacturer's instructions. A RIP I promoter fragment, spanning −412 to +155 (from transcription start site 4006, rat insulin I, GenBank Accession No. J00747) was isolated and inserted into the plasmids. This 567-bp fragment containing exon 1, intron 1 and 3 bp (GTC) of the 5′ end of exon 2 was PCR amplified from Sprague-Dawley Rat DNA by using the following PCR primers that contain a restriction site at the 5′ ends (the restriction sites are underlined):

primer 1 (XhoI)
(SEQ ID NO: 38)
5′-CAACTCGAGGCTGAGCTAAGAATCCAG-3′;
primer 2 (EcoRI)
(SEQ ID NO: 39)
5′-GCAGAATTCCTGCTTGCTGATGGTCTA-3′.

The corresponding PCR products were verified by agarose gel electrophoresis and purified by QIAquick Gel Extraction kit (QIAGEN). To confirm the sequences, direct sequencing of PCR products was performed with a dRhodamine Terminator Cycle Sequencing Kit (PE Applied Biosystems, Foster City, Calif.) on an ABI 3100 Genomic Analyzer. The PCR amplified fragments were digested with XhoI and EcoRI and then ligated into the XhoI-EcoRI sites of pDsRed-Express-1, a promoterless Discosoma sp. red fluorescence protein (DsRed) plasmid (BD Biosciences). Ligation reactions were carried out in 20 μl of 20 mM Tris-HCL, 0.5 mM ATP, 2 mM dithiothreitol and 1 unit of T4 DNA ligase. Cloning, isolation and purification of this plasmid were performed by standard procedures and once again sequenced to confirm that no artifactual mutations were present. RIP-hexokinase-1 and RIP-human insulin were made in the same manner.

In Situ-PCR for Detection of DsRed DNA.

A single pair of DsRed primers directed against the DsRed DNA was used: DsRed 125⁺ (5′-GAGTTCATGCGCTTCAAGGTG-3′; SEQ ID NO:40) and DsRed 690⁻ (5′-TTGGAGTCCACGTAGTAGTAG-3′; SEQ ID NO:41).

Immediately after sacrifice, blood was removed from the rats by 200 ml intra-arterial cooled saline followed by perfusion fixation with 100 ml of 2% paraformaldehyde and 0.4% glutaraldehyde. The pancreas was cut into 0.5 cm pieces and placed into 20% sucrose solution overnight in 4° C. and then put into OTC molds at −86° C. Frozen sections 5 μm in thickness were placed on silane coated slides and fixed in 4% paraformaldehyde for 15 min at 4° C., quenched with 10 mM glycine in PBS for 5 minutes, rinsed with PBS, permeabilized with 0.5% Triton X-100 in PBS for 10 min, and rinsed with PBS for 10 min. (A PCR DIG Prob Synthesis Kit (Roche Co.; Cat. NO: 1636090) was used.) A coverslip that was anchored with a drop of nail polish at one side was placed on the slide. The slide was then placed in an aluminum ‘boat’ directly on the block of the thermocycler. A 50 μl PCR reaction solution (0.8 units of Taq DNA polymerase, 2 μl of DsRed primers, 3 μl of DIG-dNTP, 5 μl of 10×buffer and 40 μl of water) was added to each slide and covered by the AmpliCover Disc and Clips using the Assembly Tool (Perkin Elmer) according to the manufacturer's instructions. In situ PCR was performed using Perkin-Elmer GeneAmp system 1000 as follows: after an initial hold at 94° C. (1 min), the PCR was carried out for 11 cycles (94° C. for 1 min, 54° C. for 1 min, and 72° C. for 2 min). After amplification, the slide was immersed in 2×SSC for 10 min and 0.5% paraformaldehyde for 5 min and PBS for 5 min, two times. The digoxigenin incorporated-DNA fragment was detected using a fluorescent antibody enhancer set for DIG detection (Roche), followed by histochemical staining. First, the sections were incubated with blocking solution for 30 min to decrease the non-specific binding of the antibody to pancreas tissue. Then, the sections were incubated with 50 μl of anti-DIG solution (1:25) for 1 h at 37° C. in a moisturized chamber. Then the slides were washed with PBS three times with shaking, each for 5 min and again the slides were incubated with 50 μl of anti-mouse-IgG-digoxigenin antibody solution (1:25) for 1 hr at 37° C. The slides were washed with PBS three times with shaking, each for 5 min again. The slides were incubated with 50 μl of anti-DIG-fluorescence solution (1:25) for 1 hr at 37° C. The slides were washed with PBS three times with shaking, each for 5 min again. Finally, the sections were dehydrated in 70% EtOH, 95% EtOH and 100% EtOH, each for 2 min, cleared in xylene and coverslipped.

In Situ RT-PCR for Detection of DsRed mRNA.

A single pair of DsRed primers directed against the DsRed cDNA was used: DsRed 125⁺ (5′-GAGTTCATGCGCTTCAAGGTG-3′; SEQ ID NO:40) and DsRed 690⁻ (5′-TTGGAGTCCACGTAGTAGTAG-3″ SEQ ID NO:41).

Perfusion fixed frozen sections were prepared as described above. DNase treatment was performed with 50 μl of cocktail solution (Invitrogen) (5 μl of DNase I, 5 μl of 10×DNase buffer, and 40 μl of water) on each slide, coverslipped, incubated at 25° C. overnight, and then washed with PBS 5 min 2 times.

Reverse Transcription.

First-strand cDNA synthesis was performed on each slide in a 50 μl total volume with 50 μl of cocktail solution (Superscript First-strand synthesis system for RT-PCR, Invitrogen kit #11904-018) (1 μl of DsRed727⁻ primers (5′-GATGGTGATGTCCTCGTTGTG-3′; SEQ ID NO:42), 5 μl of DTT solution, 2.5 μl of dNTP, 5 μl of 10×buffer, 5 μl of 25 mM MgCl, 29 μl of water and 2.5 μl of SuperScript II RT). A coverslip was placed and the slides incubated at 42° C. for 2 hrs, washed with PBS 5 min 2 times, rinsed with 100% ETOH for 1 min and dried.

Immunohistochemistry for Detection of DsRed Protein, Insulin, and Glucagon.

Cryostat sections 5 μm in thickness were fixed in 4% paraformaldehyde for 15 min at 4° C. and quenched for 5 min with 10 mM glycine in PBS. Sections were then rinsed in PBS 3 times, and permeabilized with 0.5% Triton X-100 in PBS for 10 min. Sections were blocked with 10% goat serum at 37° C. for 1 hr and washed with PBS 3 times. The primary antibody (Sigma Co.) (1:50 dilution in block solution) was added and incubated at 4° C. overnight. After washing with PBS three times for 5 min, the secondary antibody (Sigma Co., anti-mouse IgG conjugated with FITC) (1:50 dilution in block solution) was added and incubated for 1 hr at 37° C. Sections were rinsed with PBS for 10 min, 5 times, and then mounted.

Luciferase Assay.

To quantitate expression of the luciferase transgene, the pancreas, both kidneys, spleen and skeletal muscle were pulverized in a Polytron and incubated with luciferase lysis buffer (Promega Co.), 0.1% NP-40, and 0.5% deoxycholate and proteinase inhibitors. The resulting homogenate was centrifuged at 10,000 g for 10 minutes and 100 μl of luciferase reaction buffer (Promega) was added to 20 μl of the clear supernatant. Light emission was measured by a luminometer (TD-20/20, Turner Designs Co.) in RLU (relative light units). Total protein content was determined by the Lowry method (BCA protein assay reagent, Pierce Co.) from an aliquot of each sample. Luciferase activity was expressed as RLU/mg protein.

Human Insulin and C-Peptide Assays.

Human insulin and C-peptide were measured using a commercially available radioimmunoassay kit (Linco Research, St. Charles, Mo.). The cross-reactivity of the human assay kit with rodent proteins is <0.4%

Hexokinase I Western Blot.

Sections of whole pancreas were harvested at sacrifice (day 10 after UTMD gene delivery) from each rat and homogenized in Tris buffer. Equal amounts of protein from these tissue homogenates were subjected to electrophoresis using a 12% BioRad gel, blocked, and incubated with mouse anti-hexokinase I antibody. Immunoreactive bands were visualized with chemiluminescence substrate (ECL, Amersham, Piscataway, N.J., USA).

Statistical Analysis.

Differences in luciferase activity between experimental groups were compared by two-way ANOVA. Repeated measures ANOVA was used to evaluate the results of the time course experiment. Two-way repeated measures ANOVA was used to assess the temporal change in serum insulin and glucose between hexokinase 1-treated rats and control groups. A p value <0.05 was considered statistically significant. Post-hoc Scheffe tests were performed only when the ANOVA F values were statistically significant.

In Situ PCR for Plasmid DNA.

In-situ PCR hybridization was used to stain for the RIP-DsRed plasmid DNA, which was seen throughout the treated pancreas. Plasmid DNA was seen throughout the pancreas in a nuclear pattern, including the islets. Similar patterns of homogeneous nuclear tissue localization of the plasmid were observed in the left kidney, spleen, and portions of the liver that were within the ultrasound beam. Plasmid was not present in right kidney or skeletal muscle, organs that lie outside of the ultrasound beam. This was the case for plasmids containing either the CMV or RIP promoters, and either the LacZ or DsRed marker genes. Controls (microbubbles without plasmid or plasmid-microbubbles without ultrasound) did not show any evidence of plasmid within the pancreas. This demonstrates that the ultrasound treatment released the plasmid within the pancreas and its immediate vicinity.

In Situ RT-PCR for mRNA.

In order to confer islet specific expression, a reporter construct driven by the rat insulin-1 promoter (RIP) was delivered by UTMD. Rodents have two nonallelic insulin genes.^{16,17} The insulin II gene is expressed in pancreatic beta cells, thymus, choroid plexus and yolk sac. The insulin I gene is only expressed in pancreatic beta cells and scattered cells in the thymus¹⁸⁻²². Thus, a fragment of the rat insulin I promoter (RIP; +412 to −165 relative to the transcription start site) was used for vector construction to facilitate gene targeting to beta cells.

In-situ PCR hybridization was used to stain for DsRed mRNA, which is localized to the islet center. DsRed mRNA was seen throughout the islets, but not in the pancreatic parenchyma, indicating that the RIP promoter directed transcription of the UTMD-delivered DsRed cDNA only in the endocrine pancreas. There was no signal detected in controls, including microbubbles without plasmid, LacZ plasmid-microbubbles, or DsRed plasmid-microbubbles without ultrasound.

Demonstration of Specific Targeting of DsRed to Islet Beta-Cells by Confocal Microscopy.

Experiments were conducted to examine whether the expression of DsRed protein was confined to insulin producing beta-cells within the pancreatic islets. Expression of the DsRed protein was observed within the central core of islet cells, consistent with the known localization of beta-cells within rat islets. The DsRed protein was identified with a red filter at an excitable wavelength of 568 nm and an emission wavelength of 590-610 nm. Beta-cells were identified specifically by immunohistochemical staining with a fluorescence-tagged antibody directed against insulin at an excitable wavelength of 488 nm and an emission wavelength of 490-540 nm. Co-localization of the DsRed and insulin signals confirmed that DsRed plasmid expression was present in islet beta-cells. DsRed signal was only present in islet tissue that co-stained with anti-insulin, indicating a high degree of beta-cell specificity. In addition, there were islets identified by insulin staining that did not show DsRed expression. Examination of sections from rats infused with control microbubbles (without plasmid) or control plasmid (LacZ) did not show any detectable DsRed signal.

The location of DsRed expression relative to glucagon-producing alpha cells was also observed. The alpha cells were identified on the islet periphery by immunohistochemical staining with a fluorescence antibody directed against glucagon. Confocal microscopy showed that the DsRed signal never co-localizes with the glucagon signal, which remains bright green and located on the islet periphery.

The efficiency of islet transfection was calculated by counting the number of DsRed-positive islets divided by the total number of islets (anti-insulin positive)×100. Transfection efficiency was significantly higher for islets treated with the RIP-DsRed compared to CMV-DsRed plasmid (67±7% vs 20±5%, F=235.1, p<0.0001). As noted above, islets treated with control microbubbles (no plasmid or LacZ plasmid) did not show any detectable transfection.

Taken together, these data demonstrate that coupling of UTMD with plasmids in which transgene expression is controlled by RIP results in efficient delivery of genes in a highly targeted, if not exclusive fashion to islet β-cells in living rats.

Quantitative Luciferase Gene Expression.

Experiments were conducted to quantitate gene expression in the pancreas compared to other organs within the ultrasound beam (left kidney, spleen, liver) and outside the ultrasound beam (right kidney, hindlimb skeletal muscle). Rats were sacrificed at day 4 after UTMD and luciferase activity measured in each organ and indexed for protein content as RLU/mg protein. Three groups of rats (n=3 rats per group) were included in the experiment: animals that received CMV-luciferase microbubbles, fed on normal chow and water, animals that received RIP-luciferase microbubbles fed on normal chow and water, and animals that received RIP-luciferase microbubbles and received normal chow plus water supplemented with 20% glucose. Animals were provided these diets for 4 days prior to sacrifice. In animals that received CMV-luciferase, a low level of activity was detected in all organs within the ultrasound beam. No activity was detected in skeletal muscle or right kidney, which lie outside the ultrasound beam. By ANOVA, the difference in pancreatic luciferase activity between organs was statistically significant (F=42.4, p<0.0001), due to the markedly higher activity in pancreas compared to the other organs. Of particular importance, the RIP-luciferase plasmid increased pancreatic activity by 100-fold compared to liver (298±168 RLU/mg protein vs 2.9±0.8 RLU/mg protein), indicating that this technique largely circumvents the problem of hepatic uptake seen with viral vectors.

The RIP-luciferase plasmid increased pancreatic luciferase activity by 4-fold compared to CMV-luciferase (298±168 RLU/mg protein vs 68±34 RLU/mg protein, p<0.0001). Glucose feeding further increased pancreatic luciferase activity by 3.5-fold over RIP-luciferase alone (1084±192 RLU/mg protein vs 298±168 RLU/mg protein, p<0.0001), indicating that the RIP-luciferase transgene was appropriately regulated by glucose following delivery to islets by UTMD.

Surprisingly, glucose feeding also caused regulation of luciferase expression in the left kidney compared to RIP-luciferase alone (172±102 RLU/mg protein vs 53±23 RLU/mg protein, p=0.0057), suggesting that the rat insulin promoter responds to glucose even when localized to the kidney.

Time Course of Gene Expression by UTMD.

In a separate group of rats, the time course of gene expression by UTMD was measured using the RIP-luciferase plasmid. Luciferase activity was measured by sacrificing 3 rats each at 4, 7, 14, 21, and 28 days after UTMD. Luciferase activity dropped by half from day 4 to day 7 and was nearly undetectable by day 21 (F=234, p<0.0001).

Delivery of Human Insulin Gene by UTMD.

In the first of two experiments to test the efficacy of the UTMD procedure to alter biological function of pancreatic islets, UTMD was evaluated as a method for delivery of the human insulin gene to rat β-cells. To this end, a plasmid containing the human insulin gene under control of the RIP promoter was used. Six rats received RIP-human insulin by UTMD and six rats served as controls (3 sham and 3 treated with RIP-DsRed). Serum measurements of human insulin, human C-peptide, and glucose were compared at baseline, day 5, and day 10 after UTMD. Human insulin and C-peptide were “detectable” (due to cross-reactivity with rat insulin and rat C-peptide) in the controls, but delivery of the RIP-human insulin plasmid by UTMD caused significant increases in both analytes (p<0.0001 for both human insulin and C-peptide). In addition, serum glucose decreased from 130±11 mg/dl at baseline, to 102±10 mg/dl, and 116±9 mg/dl at 5 and 10 days, respectively (p=0.02).

Regulation of Insulin Secretion and Circulating Glucose Levels by UTMD-Mediated Delivery of the Hexokinase-1 Gene.

As a second demonstration of the efficacy of the UTMD method, a gene was chosen that must cause a significant change in β-cell metabolic function in order to cause a change in whole-animal phenotype. Previous studies have demonstrated that overexpression of low Km hexokinases (e.g., hexokinase I) results in a left-shift in the glucose dose response for insulin secretion, due to increased stimulus/secretion coupling at low glucose^{3,23}. Experiments were carried out to test whether UTMD-mediated delivery of a RIP-hexokinase plasmid could alter endogenous insulin production and glucose homeostasis in normal rats. Six rats were infused with microbubbles containing the RIP-hexokinase I plasmid, whereas controls included rats infused with RIP-DsRed-containing microbubbles (n=3) and sham-operated normal rats (n=3). Serum measurements of glucose and insulin were obtained at baseline, and at days 5 and 10 after UTMD. There was no significant change over time in serum insulin or glucose levels in the RIP-DsRed or sham surgery control groups. In contrast, serum insulin increased by 4-fold at day 5 and remained elevated at day 10 in the RIP-hexokinase I-treated groups (F=11.5, p=0.0033 by repeated measures ANOVA, treated vs controls). Correlating with the increase in insulin, serum glucose levels decreased by nearly 30% in the RIP-hexokinase I-treated rats at day 5 (F=19.8, p=0.0005 by repeated measures ANOVA, treated vs controls), and then remained low out to day 10. Further evidence of highly efficient delivery of the hexokinase I gene to pancreatic islets by UTMD is provided by immunoblot analysis of hexokinase I protein levels in islets isolated at day 10. These data show a clear increase in immunodetectable hexokinase I protein in islets of all 6 rats subjected to UTMD with the RIP-hexokinase I plasmid relative to either control group. In sum, these data clearly demonstrate the use of UTMD for high efficiency gene delivery to pancreatic islet β-cells in living animals.

Safety of UTMD.

Histologic sections of the pancreas did not reveal any evidence of inflammation or necrosis after UTMD. In 4 rats, serum amylase and lipase were measured at baseline, 1 hr, and 24 hrs after UTMD; values were normal and did not increase with UTMD. Rats subjected to UTMD gained weight normally and demonstrated no abnormal behaviors. Moreover, rats that received the RIP-DsRed plasmid experienced no significant changes in circulating glucose or insulin levels, suggesting maintenance of normal metabolic homeostasis.

The approach described provides safe and efficacious delivery of DNA constructs to beta-cells with several advantages: 1) No viral vectors are required for efficient gene transfer, limiting concerns for inflammatory responses or insertional mutagenesis; 2) Use of the RIP promoter in these plasmid constructs provides a remarkable degree of beta-cell specificity within islets, with little to no expression of the DsRed reporter gene in glucagon producing alpha cells; 3) The microbubbles loaded with plasmid can be delivered via the systemic circulation, obviating the need for invasive surgery such as would be required for local delivery to pancreatic vessels; and 4) There was no evidence of pancreatic damage arising as a result of microbubble infusion and local application of ultrasound in the pancreas.

### References for Example 3

1. Winer, N., Sowers, J. R. (2004) Epidemiology of diabetes. J. Clin. Pharmacol. 44, 397-405
2. Yamaoka, T. (2001) Gene therapy for diabetes mellitus. Curr Mol Med 1, 325-337.
3. Becker, T., Beltran del Rio, H., Noel, R. J., Johnson, J. H., & Newgard, C. B. (1994) Overexpression of hexokinase I in isolated islets of Langerhans via recombinant adenovirus: enhancement of glucose metabolism and insulin secretion at basal but not stimulatory glucose levels. J. Biol. Chem. 269, 21234-21238.
4. Flotte, T., Agarwal, A., Wang, J., Song, S., Fenjves, E. S., Inverardi, L., Chesnut, K., Afione, S., Loiler, S., Wasserfall, C., et al. (2001) Efficient ex vivo transduction of pancreatic islet cells with recombinant adenoassociated virus vectors. Diabetes 50:515-520.
5. Wang, A. Y., Pend, P. D., Ehrhardt, A., Storm, T. A., & Kay, M. A. (2004) Comparison of adenoviral and adeno-associated viral vectors for pancreatic gene delivery in vivo. Human Gene Ther. 15, 405-413.
6. Ayuso, E, Chilton, M., Agudo, J., Haurigot, V., Bosch, A., Carretero, A., Otaegui, P. J., & Bosch, F. (2004) In vivo gene transfer to pancreatic beta cells by systemic delivery of adenoviral vectors. Human Gene Ther. 15, 805-812.
7. Koehler, D. R., Hannam, V., Belcastro, R., Steer, B., Wen, Y., Post, M., Downey, G., Tanswell, A. K., & Hu, J. (2001) Targeting transgene expression for cystic fibrosis gene therapy. Mol. Ther. 4, 58-65.
8. Tirone, T. A, Fagan, S. P., Templeton, N. S., Wang, X. P., & Brunicardi F. C. (2001) Insulinoma-induced hypoglycemic death in mice is prevented with beta-cell specific gene therapy. Annals. Surg. 233, 603-611.
9. Tirone, T. A, Wang, X. P., Templeton, N. S., Lee, T., Nguyen, L., Fisher, W., & Brunicardi F. C. (2004) Cell-specific cytotoxicity of human pancreatic adenocarcinoma cells using rat insulin promoter thymidine kinase-directed gene therapy. World J. Surg. 28, 826-833.
10. Shohet, R. V. Chen, S-Y., Zhou, Y-T., Wang, Z-W., Meidell, R. S., Unger, R. H., & Grayburn, P. A. (2000) Echocardiographic destruction of albumin microbubbles directs gene delivery to the myocardium. Circulation 101, 2554-2556.
11. Chen, S. Y., Shohet, R. V., Bekeredjian, R., Frenkel, P. A. & Grayburn, P. A. (2003) Optimization of ultrasound parameters for cardiac gene delivery of adenoviral and plasmid deoxyribonucleic acid by ultrasound-targeted microbubble destruction. J. Am. Coll. Cardiol. 42, 301-308.
12. Bekeredjian, R., Chen, S-Y., Frenkel, P., Grayburn, P. A. & Shohet, R. V. (2003) Ultrasound targeted microbubble destruction can repeatedly direct highly specific plasmid expression to the heart. Circulation 108, 1022-1026.
13. Korpanty, G. Chen, S-Y., Shohet, R. V., Ding, J-H., Yang, B-Z., Frenkel, P. A., & Grayburn, P. A. (2005) Targeting of VEGF-mediated angiogenesis to rat myocardium using ultrasonic destruction of microbubbles. Gene Ther. 12, 1305-1312.
14. Bekeredjian, R., Grayburn, P. A., & Shohet, R. V. (2005) Use of ultrasound contrast agents for gene or drug delivery in cardiovascular medicine. J. Am. Coll. Cardiol. 45, 329-335.
15. Frenkel, P. A., Chen, S-Y., That, T., Shohet, R. V. & Grayburn, P. A. (2002) DNA-loaded albumin microbubbles enhance ultrasound-mediated transfection in vitro. Ultrasound Med. Biol. 28, 817-822.
16. Wentworth, B. M., Schaefer, I. M., Villa-Komaroff, L., Chirgwin, J. M. (1986) Characterization of the two nonallelic genes encoding mouse preproinsulin. J. Mol. Evol. 23, 305-312.
17. Deltour, L., Leduque, P., Blume, N., Madsen, O., Dubois, P., Jami, J., Bucchini, D. (1993) Differential expression of the two nonallelic proinsulin genes in the developing mouse embryo. Proc. Natl. Acad. Sci. U.S.A. 90, 527-531.
18. Throsby, M., Homo-Delarche, F., Cheyenne, D., Goya, R., Dardenne, M., Pleau, J-M. (1998) Pancreatic hormone expression in the murine thymus: localization in dendritic cells and macrophages. Endocrinology. 139, 2399-2406.
19. Chentoufi, A. A., Polychronakos, C. (2002) Insulin expression levels in the thymus modulate insulin-specific autoreactive T-cell tolerance: the mechanism by which the IDDM2 locus may predispose to diabetes. Diabetes. 51, 1383-1390.
20. Chentoufi, A. A., Palumbo, M., Polychronakos, C. (2004) Proinsulin expression by Hassall's corpuscles in the mouse thymus. Diabetes. 53, 354-359.
21. Lamotte, L., Jackerott, M., Bucchini, D., Jami, J., Joshi, R. L., Deltour, L. (2004) Knock-in of diphtheria toxin A chain gene at Ins2 locus: effects on islet development and localization of Ins2 expression in the brain. Transgenic Res 13, 463-473.
22. Park, S. Y., Wang, X., Chen, X., Powers, A. C., Magnuson, M. A., Head, W. S., Piston, D. W., Bell, G. I. (2005) Optical imaging of pancreatic beta cells in living mice expressing a mouse insulin I promoter-firefly luciferase transgene Genesis. 43, 80-86
23. Epstein, P. N., Boschero, A. C., Atwater, I., Cai, X., & Overbeek, P. A. (1992) Expression of yeast hexokinase in pancreatic beta cells of transgenic mice reduces blood glucose, enhances insulin secretion, and decreases diabetes. Proc. Natl. Acad. Sci. U.S.A. 89, 12038-12042.
24. Yamamoto, K. Hashimoto, H., Tomimoto, S., Shintani, N., Miyazaki, J., Tashiro, F., Aihara, H., Nammo, T., Li, M., Yamagata, K., et al. (2003) Overexpression of PACAP in transgenic mouse pancreatic beta-cells enhances insulin secretion and ameliorates streptozotocin-induced diabetes. Diabetes 52, 1155-1162.
25. Hara, M. Wang, X., Kawamura, T., Bindokas, V. P., Dizon, R. F., Alcoser, S. Y., Magnuson, M. A., & Bell, G. I. (2003) Transgenic mice with green fluorescent protein-labeled pancreatic beta-cells. Am. J. Physiol. Endocrinol. Metab. 284, E177-E183.
26. Marron, M. P., Graser, R. T., Chapman, H. D. & Serreze, D. V. (2002) Functional evidence for the mediation of diabetogenic T cell responses by HLA-A2.1 MHC class 1 molecules through transgenic expression in NOD mice. Proc. Natl. Acad. Sci. U.S.A. 99, 13753-13758.
27. Cebrian, A. Garcia-Ocana, A., Takane, K. K., Sipula, D., Stewart, A. F., & Vasavada, R. C. (2002) Overexpression of parathyroid hormone-related protein inhibits pancreatic beta-cell death in vivo and in vitro. Diabetes 51, 3003-3013.
28. Hussain, M. A., Miller, C. P, & Habener, J. F. (2002) Brn-4 transcription factor expression targeted to the early developing mouse pancreas induces ectopic glucagon gene expression in insulin-producing beta cells. J. Biol. Chem. 2002 277, 16028-16032.
29. Tuttle, R. L. Gill, N. S., Pugh, W., Lee, J. P., Koeberlein, B., Furth, E. E., Polonsky, K. S., Naji, A., & Birnbaum, M. J. (2001) Regulation of pancreatic beta-cell growth and survival by the serine/threonine protein kinase Akt1/PKBalpha. Nat. Med. 7, 1133-1137.
30. Thorens, B., Guillam, M. T., Beermann, F., Burcelin, R. & Jaquet, M. (2000) Transgenic reexpression of GLUT1 or GLUT2 in pancreatic beta cells rescues GLUT2-null mice from early death and restores normal glucose-stimulated insulin secretion. J. Biol. Chem. 275, 23751-23758.
31. Stellrecht, C. M., DeMayo, F. J., Finegold, M. J., & Tsai, M. J. (1997) Tissue-specific and developmental regulation of the rat insulin II gene enhancer, RIPE3, in transgenic mice. J. Biol. Chem. 272, 3567-3572.
32. Pawelczyk, T., Podgorska, M. & Sakowicz. M. (2003) The effect of insulin on expression level of nucleoside transporters in diabetic rats. Mol. Pharmacol. 63, 81-88.
33. Zhang, S. L., Chen, X., Wei, C. C., Filep, J. G., Tang, S. S., Ingelfinger, J. R., & Chan, J. S. (2002) Insulin inhibits dexamethasone effect on angiotensinogen gene expression and induction of hypertrophy in rat kidney proximal tubular cells in high glucose. Endocrinology 143, 4627-4635.
34. Zhan, Y., Brady, J. L., Johnston, A. M., & Lew, A. M. (2000) Predominant transgene expression in exocrine pancreas directed by the CMV promoter. DNA and Cell Biol. 19, 639-645.
35. Zeng, Y., Yi, R. & Cullen, B. R. (2003) MicroRNAs and small interfering RNAs can inhibit mRNA expression by similar mechanisms. Proc. Natl. Acad. Sci. U.S.A. 100, 9779-9784.
36. Bain, J., Schisler, J. C., Takeuchi, K., Newgard, C. B., & Becker, T. C. (2004) An adenovirus vector for efficient RNAi-mediated suppression of target genes in insulinoma cells and pancreatic islets of Langherhans. Diabetes 53, 2190-2194.

### Example 4

### Similarities and Critical Differences in the Nkx6.1 and Pdx-1 Transcriptional Network in Adult β Cells

To identify similarities and differences in the Nkx6.1 [1] and PdX-1 [3] transcriptional networks, the following studies were undertaken.

Recombinant adenoviruses were prepared, containing the cDNAs encoding Nkx6.1, Pdx-1, or as a control, bacterial β-galactosidase. Treatment of rat islets with these viruses resulted in an approximate 5-fold increase in Nkx6.1 and Pdx-1 protein expression. Overexpression of either Nkx6.1 or Pdx-1 resulted in a striking increase in islet cell replication, as indicated by a 6-7-fold increase in [³H]-thymidine incorporation. Using immunocytochemistry and cell counting, this increase in replication corresponded to a 30% increase in β-cell mass over a period of 4 days. In contrast to the very similar effect of Nkx6.1 and Pdx-1 on islet replication, only Nkx6.1 overexpression caused an enhancement in GSIS in rat islets.

The expression of a panel of cell cycle, mitogenic, and functional genes was also compared by real-time PCR in rat islets overexpressing Nkx.6.1 (AdCMV-Nkx6.1), Pdx-1 (AdCMV-Pdx-1) or β-galactosidase (AdCMV-βGal). In these experiments, there was a substantial overlap in the cell cycle gene targets by Nkx6.1 and Pdx-1, both of which elicited upregulation of cyclins A2, B1, E1, and a host of regulatory kinases and accessory molecules, with little or no effect on cyclin D2 or Cdc25a, which have been previously identified as being downstream of Akt/protein kinase B activation [4,5]. Thus, these experiments suggest that these two genes activate similar pathways in eliciting β-cell proliferation.

Nkx6.1 GSIS Pathway.

To determine if downstream components of the Nkx6.1 transcriptional pathway distinct from those regulated by Pdx-1 may represent novel regulators of GSIS in β-cells, the following experiments were conducted. Replicate (n=4) cDNA microarray studies were performed in primary rodent islets overexpressing either Nkx6.1 or Pdx-1, with normalization to AdCMV-βGal-treated islets in both cases. As summarized in Table 7, comparison of the microarray results revealed a number of genes that were upregulated by Nkx6.1, but not by Pdx-1 by ≧2-fold, as well as genes that were decreased in expression by 50% in response to Nkx6.1 but not Pdx-1 expression. These data provide a focused list of candidates that may underlie the differential ability of Nkx6.1 overexpression to enhance GSIS relative to Pdx-1.

From this list, siRNA-targeted knockdowns have been performed for some of the genes in glucose responsive INS-1-derived cells (the 832/13 cell line (6)) using AMAXA-based nucleotransfections, and a role for 4 of these genes has thus far been verified in regulation of GSIS (glutamate receptor, malate transporter (DIC), cytosolic isocitrate dehydrogenase [7], and adenosine receptor).

The adenosine receptor Adora1 is expressed at higher levels in robustly glucose responsive INS-1-derived cell lines compared to poorly responsive lines, similar to findings regarding expression of Nkx6.1 in these cell lines [2]. Furthermore, siRNA-mediated suppression of Adora1 significantly diminished GSIS in glucose responsive INS-1-derived lines. siRNA-mediated suppression of another Nkx6.1 target gene, the ionotropic glutamate receptor, Grin2c, similarly resulted in a marked reduction in GSIS, without affecting cAMP-potentiated insulin release, indicating that the general exocytic machinery is not dependent upon Grin2c expression.

### References for Example 4

1. Schisler et al., The Nkx6.1 homeodomain transcription factor suppresses glucagon expression and regulates glucose-stimulated insulin secretion in islet beta cells. Proc Natl Acad Sci USA, 2005. 102(20): p. 7297-302.
2. Schisler et al., The Homeodomain Transcription Factor Nkx6.1 Stimulated Proliferation and Preserves Insulin Secretion in Mature Human and Rodent Pancreatic Islet beta-cells. in submission.
3. Jonsson et al., Insulin-promoter-factor 1 is required for pancreas development in mice. Nature, 1994. 371(6498): p. 606-9.
4. Bernal-Mizrachi et al., Islet beta cell expression of constitutively active Akt1/PKB alpha induces striking hypertrophy, hyperplasia, and hyperinsulinemia. J Clin Invest, 2001. 108(11): p. 1631-8.
5. Tuttle et al., Regulation of pancreatic beta-cell growth and survival by the serine/threonine protein kinase Akt1/PKBalpha. Nat Med, 2001. 7(10): p. 1133-7.
6. Hohmeier et al., Isolation of INS-1-derived cell lines with robust ATP-sensitive K+ channel-dependent and -independent glucose-stimulated insulin secretion. Diabetes, 2000. 49(3): p. 424-30.
7. Ronnebaum et al., A pyruvate cycling pathway involving cytosolic NADP-dependent isocitrate dehydrogenase regulates glucose-stimulated insulin secretion. J Biol Chem, 2006. 281(41): p. 30593-602.

The foregoing is illustrative of the present invention, and is not to be construed as limiting thereof. The invention is defined by the following claims, with equivalents of the claims to be included therein.

TABLE 2
Human, mouse and rat Nkx6.1 promoter sequences

Human Nkx6.1 promoter (SEQ ID NO: 20)
CATCATCTGACAGATAAATATTCCTATTAAACGGATTCAGTTTTCAGCG
AATTGAGTAACCCATAAATGATAATGAACGCGGTGGGAAGCGACGGGCG
GGGGGGAACTCGGGAATGAAAAAAAAAATAAAGTGGAGGAGAAAGAACA
GAAAAGGAAAGCAGGAGGTGGAAAGATGGAAGAGGACGATCCTTTGGCC
TACAAGGGGATTAAGGACATCTATAAGGCTTAAGGAGCAACAAATTAAT
TTACACAATTCTGGGAGAGCCCAGATGGCCTTTAATTAATCCCTTCAAA
AGAAGGAGCCAGGCCAGGGCTGCGCCGGCTGCCTGCTCCATTAGCTCCA
TTTTACAAGGGACCAGACTTGGTTCGAGGTGAGGCGCCCTCCAGAGCTG
GTGGGGGAAGGGGATAGGATGACGCGAGCGGGCTAGTGGGGAAGCAAGG
GAAGAATATGAACTGCTTTTCCATAAAAGGGCTGAGTTTTCATTATTCC
TCTCTTTAAAAAGTAATACCCTCTTCGTCTCTGCTTCCCCCTCCCCTTT
CTCATTTTATTTAGCACAATTAATTGAGGCGGCCACTGGCCCCAGCGCG
GAACCGCACCACTCACCAGCTCCCGCCCCTCCTGGCCCCGCCCACAGGA
GAAAGAAGTAGGGAGCGGGAGGGGACTAGGCGGGCGCGGCCCTACGCCT
GGCCCGCCTCAGCCAATCAGAGGGTGCGGCGCCCCCGAGTGGGCGAGCC
CCAGGGGCGACGCAAGGATCGAGGCGGCGAGCTATTGGACACGGTGGTT
ACGCCCCCGGCCTGCGCCCGGCTCGCCGGCCCCCGCAGCCTCGGAGTGA
CGTCCCTCAAAGTTCTCATTTTGGTCCCCCACTTCCCCCTCCCTTTCGT
CCCCCAGCTAAAGAGGGGTAGGGAGTGATGCAAATGTTTTATTACCTTT
GAGAGCTTCATCTGAACTGTCAGGCCCGGAGGGAGAGAGGAAAGGAGAG
AAAGAGCGGAGGAGCCGCGGAGAGGGTCAGTTTGGCCAGAGGACAGGAC
TTAGAAGACCGAAGCCTGGGAAGCCGCGAAGAAAATGCCAGAGAGGAGA
GTCAAAGGCTGAGAGTGAGAGGGAGAGAGGGAGCGGGGGTGGGGCGGGG
GTCGCCGGGCCCGTTTGCAGAAGTGGACTGACGAGCGGCGCCGAAACCC
AGCCGTCAGACTTTTCACACTTAGTCTTTTGTTTTTCTGTGTTTTTCCT
CCCCCTTTTTCTTTTCAATATTGCAACTCCAGTGCCCCGTGGGCCAGAG
GGCAAGGCGGGTGGAGGTGAGGACCTGGGAGCCGCGGGGATCCGTGGCA
CTGCCCTTTCTGGCGCAGCAGCCCGGGGCAGCGTGGGCGGAGGAAGCCC
GCACAGAGGCTAGATCTCCCGCGGGCTGGATGCGCTTTCTCCCCGGGCA
CAGTGAGCGTCGAATGCGAATCAGCCGCGCGACCGAAAGAGCAGAGCAT
CCCAGTAAGATCAGAGGAGCGCCACGGGCTGCACAAGGCGTCCTTTGAA
CCTCCCCAAAGAAAGCAAGCCACCCCCACCCTCCAACTTCAAAGTGGAG
ATTCGGCAACTAACTTTGCTACAAACTCTCCGGAGCCAGCCTGGGTTTT
GTTTTGCTTATTTCCCGGGGGCAGAAGATGAGAAGTAGCGCACTTTGAA
CAGCTAGGAAAAGTGAGGAAGAGAGAATAGCCAGGGATCGAATCTAGGA
CTCGCGGAACGAAAGGACTGCCTAGCCCGCCGGGACGCCTGCTTTTCTC
GGCGAGCTGCCGCCTCCCGCGTGGAGGGTTTGGACATCTCTGCTGCGCA
GCTAGGCGAGCAACTCCCGGCAGCGGCATTTTTGGTTCAGTTGGCAGCT
CGCCTCCGGGCGCGCCGAGTGCCTCTCCGCTCGCGCCCTCGGCGCTTCC
GGCTCCTCTGAGCCCCGCGGGGGGCACCAGCCAGCGCCCTCGCTGCAAG
GCTACGGTCTCCGGCGTGGCCGTGGGATG
Mouse Nkx6.1 promoter (SEQ ID NO: 21)
CTGAGCGAATGAAAGGAGCAGGCGATGATTAACCCCTAGGCTTTACTCT
CACACGTCAAACTCAACCTCAAGTTGACAACACTCAAGCTTTGGAGAAG
TAAGGGGACCGACCAGTTTAAGGCCTCCTGCTTGACATTCAGAGTCAAA
CTCCCTGGCGCTGCTCAGTTTTAATTCCTGGGTCATTTATCGCCTATTT
CTGTTTTCTGAACCTTAAATTTGGACTCAATAATATGATGCAAATCTCT
GCTGTGACACTTCCCGCCCCCCCCCCCCCCACTGGTGTATCAACTGCCC
GATTTCTCAAGATCGACCAAAGAGGTTTTTTCCTTGGTTTTGGTCAACC
CTGAGCAGACCTTAAAGATCGGCCAGAGGGAGCAAAGCCCTTTTGACCA
TCGCTCCCAATGTCAGCCTAGAAGTCGGTCGTCTCTAGTTTACTCAACT
ACCCGAGTTGAGAGCTTGACCAGACTTTCCAACAGTTACCTGTCTTCCC
CCGAGGTATTCCTCTATCTAAAGTTGTCCTGTGAATTTTAGTGATCCTG
CCTCATAAATCCAACTAATAATAATAGAGGGAGGATTTTAAAAAAATAA
TTATCTCATTTCTGTTAGGTTTAGACACCACGCAGGAGATAAATATTCT
CATTAAGCTGATTTCATCCCCAGAGTACTGAGCCCCCTCATAAGTGATA
ATGATCTAGGGAGTGGGAGAGCGAAGACAAGAACGGAGAAAGAACAGAA
AAGAGCAGGAGACGGAAAGATGGTGAAGGGTGACCCTTAGGCCTGCGAG
GGGTTTAAAAACATCTACGGGCTTAAGGAACAACAAATCAATTTACACG
ATTCTGGAAGAGCCCAGAGGGCCTTTAATTAATCCCTTCAAAAGAAGGA
AGTCGGCCTGGGATATGCCTCCTGCCTGCTCCATTAGCTCCCTTTTCGC
AAGGGTCCAGACACCGTTGGAGGTGGGCGCCGCGCGCGAGCTGGGGGAG
GATGACGCGAGCTGGCGTGGGCGGAAGAGCCGCACTTAAACTGCTTTTC
CATAGAAGGGCTGGATTTTCATTATTCCTCTCTTTAAAAAGTAATGCCC
TCTTCGTCCCTGCTCCCTCCTTCTCCTTTCCATTTTATTTTGCACGATT
AGTTGAGCCGGCCGCTGGCTCTAGACTGGAACCACTCTTTTCGCCAGGC
CCCTCCCCTCTTGGCTCCGCCCAAGTGAAGCTGGGGCGGGGACTAGGAG
GGCGCGTCCTTATGGCTCCCTAGTCTCAGCCAATCAAAAGGTGTGGCGC
TCCCAGGTGGGCGTGTTCTAGGAGCGACGCCTTGCCCAAGCTGAGCGCT
ATTGGAGGCGGTGTTTACGCCCAGGACCCGGGCCCCGCTCCTCAGTCCC
GCCCCGCCGAGCCGCCCCGGAATGACGTCCTCGAAAGTTCTCATTTTGG
CCCCCCACCTCCCCTCCCTTGCGTCCCCCAGCTAAAGAGAGGCAGGGAG
GGGTGCAAATATTTTATTACCTTTGAGAGCTTCATCCGAACTGTCAGGC
CCAGAGGGAGAGAGGAGAGAAGGGAAGAGCCGCCGAGAGGGTCAGTTTG
GCCAGAGGACAGGGCTTGGAAGAGCCAAGCCTGGAAAGCCAAGGAGAAA
TGCCAGAGAGGCAAGAGAAAGGCGGAGAGTGAGAGGAAGAGAGGGCGCC
AGGGGGTGGGACGGGGGGGGGTCTCCCGGCCGGCTTGCAGAAGAGGACG
GACGATCGGAACCGGCCCGTCAGACTTTTTCCATACTTAGGTTTATTTT
CTTTTATCCTTTTCGATCCGGCTAGTCCGGCGCCGCGTGGCTCGGAGGG
GAAGTAGGCTCGTGGGCGCACCAAGTCCCCAGCCGCCGGAGCGGTGTCA
CTGACTGCCCTTTCTGGCGCAGTAACGCCGGGCCCGAGGCAGTGGCGGG
CGCGATACCCGCGCCGGCCGTTTGTGCTCTGTCCCCCGGGCGAGCGGTG
AGTGCGACTCAGCAGCGCGGCCGCCGGGAGAGCGGAGCGTCCGAGCGAG
ATCAGAGGCGCGCACCGGGCGGAACGCCGCCCGCTTTGAAGCTCCCCCA
GGCGAGCGAGCCGGCCCCCGCCCTCCTACATCAAAGCGAACGCTCCGCG
CCTCCCAACCTTGTTGCAAACTCTCTGGGTCGGCTGCGGGGTACGTCTT
GCTGATTTCCCGCGGGGGTGGAGAAGATGAGAAGCAGAGCGCTCTGAGC
CGGGAACGAGGGACCAGCGCCTGGGATCGAATCCGGGACTCCCGAAGCC
GAGGAAGCGCTGAGCCCGCCCGCGCCCCCGCAGCCCTCGCCCCTGCCGC
CTCCCGCGGGGCGTTTGGACATTTTTGCTGCGCAGCTCCCGGAGCCCGC
GGCCGATCCACACTTCGCTTGCGCGCGCCCCCGGCACCTCGGGTTCTCC
CGAGCCCCGGCGGGGCCACCGACCTGCGTGGCTGCGGGTTCGGGTCTGG
CTGTGGGATG
Rat Nkx6.1 promoter (SEQ ID NO: 22)
GCCTATGAAGCGGCTCCATGAAATTGGCCGCCTGCTCTTCTCTGGTTTA
AGCTGCAGAAGCCTTTCCTGGCTTGGAAGTGCAGACAGAACCCACTCCA
GCTCTCTCCCGAGCCTAGAGGCTGGCAGGCCCCTTAGCGTATCTCCCAT
TCATTCAGAACCCCACCTCTCCTAGGGTCAGATCCTAAATTCTACCGGG
TTGATAGCATTAACCAAGTCTTGAGGCTAATAAAGGTGGGAGAGCTGGA
GGAAGACAGATATCATCTTCCCCCACCCCCGACCCGACTCCGAGTGAAA
GAAAGGGGCTAACGATGATTAACCCCTAGACTTTCCGCTCACACCTCTA
ATCCAACCTCAGGTTGACACCCGTGAAAGACCCAAGCTTTGGCGGAGGT
AAGAGGACAGGATCAAGGCCTCCTACTGGAAGCACCTTGACATTCAGAG
TCAAACTCCCCGGGGCGCTGCTCGGTTTTTATTCTTGGGTCATTTATTG
CATCTATTTCTGTTTCCTGAACCTTAAATTTGGACTCAATGATATGATG
TAAATCTCTACTGTGACACTTCCCGCCCCCACACTGGTGTATCAACCGC
CTGATTTCTCAGGATCGACCAAAGAGGTTTCTTTTTTCTCCTCAGTTTA
GGTCAACCCTGACCAGACCTTAAAGATCAAGCAGAGGGAGCAAAGCCAT
TTTGACCATCGCTCCCAATCCTAGCCCAGAAGTCAGTAGTCTCTTGCTC
ACTCAAATCCCCAGAGTTGTGGGCTTGACCGGACTTTCCAGCAGCTTCC
TGTCTCCCCCGAGGTATTCCTCTGTCTAAAGTTGTCGCGTGAATTTTAG
TGATCCTGCCTCGTGTGATCCAACTAATAATAATAGGAGGAATTTTAAA
AAATCATCTCATTTTTCTTAGGGTTAGACACCACGCAGGAGATACATAG
TCTCGTTAAGTTGATTTCATCCCCGGAGTAGTGAGCCCCCTCGTGAGTG
ATAATGATCTAGGGGGAGGGAGAGTGAAAACAAGAACGGAGGAAGAATA
GAAAAGAGCAGGAGATGGAAAGATGGGCGAGGATGGCCCTTAGGCCTGC
GAGGGGATTTAAAAACATCTATGGGCTTAAGGAGCAACAAATCAATTTA
CACAATTCTGGAAGAGCCCAGATGGCCTTTAATTAATCCCTTCAAAAGA
AGGAACCCTGCCTGGGACGTGCCTCCTGCCTGCTCCATTAGCTCCCTTT
CCGCAAGGGTCCAGACACCAGTCGAGGTAAGGTGCTGCGAGCTAGTGGG
GGAAATGTGGGAGGACGGAAGAGATGAACTTAAACTGCTTTTCCATAAA
AGGGCGGTCTTTTCATTATTCCTCTCTTTAAAAAGTAATGCCCTCTTCG
TCCCTGCTCCCTCCTTCTCTTTTCCATTTTATTTTGCACAATTAATTGA
GCCGGCCGCTGTCCCTAGGCTGGAACCACTCTTTTGGCCAGGCCCCTCC
CCTCTTGGCTCCGCCCAAAGAAAGCTGGGGCGGGGATTAGGCGGGCGCG
CCCTTGCGGCTCCCTAGTCTCAGCCAATCAAAAGGCATGGCGCTCCCCG
GTGGGCGTGTCCCAGGAGCGACGCCTCGCTGAGGCTGAGCGCTATTGGA
TGCGGTGGCTACGCCCAGGCTCTGCACTCCGACCCTCAGTCCCGCCTCT
CGGAGCCGCCCCTGAGTGACGTCCTCGAAAGTTCTCATTTTGGCCCCCC
GCCTCCCCTCCCTCGCGTCCCCCAGCTAAAGAGCGACAGGGAGGGGTGC
AAATGTTTTATTACCTTTGAGAGCTTCATCCGAACTGTCAGGCCCGGAG
GGAGAGAGGAGAGAAGGGAAGAGCCGCCGAGAGGGTCAGTTTGGCCAGA
GGACAGGGCTCGGAAGAGCCAAGCCTGGAAAGCCAAGGAGAAAGGCCAG
AGAGGCGAGAGAAAGGCGGAGAGTGAGAGGAAGCGAGGGCGCCGGGGGG
GTGGGGCGGGGGACTCGGGGCCGGCTTGCACAAGCGGACGGACGATCGG
AACCGGCCCGTCAGACTTTTCCCACTTAGGTTTATTTTCTCTTTCTTCC
TTTTTCGGTCCCGCTACTCCGGCGCCGCGTGGCCCGGAGGGGAAGCAGG
CGCGTGGGCGCGCCAAGTGCCCAGCCGCCGGAGCGGTGCCGCCACTGCC
CTTTCTGGCGCAGTAACGCCGGTCCCACGGCGGTAGCGGGCGCGATCTC
CGCGAGAGCCGTTTGCGCTCCATCCCCTGGGCGAGCGGCGAGTGCGACT
CAGCTGCGCGGCCGCCGGGAGCGCGGAGCGTCCGAGCGAGAACAGAGGC
GCGCACCGGGCGGAACGCGGCCCGCTTTGAAGCTCCCCTAGGCGGGCGA
GCCGGCCCCCACCCTCTTACATCAAAGCGATCGCTCCGCGCCTCCCAAC
CTTGTTGCAAACTCTCTGGGCCAGCCCCGGGTACGTCTTCCTGATTTCC
CGCGGGGGTGGAGAGATGAGAAGCAGAGCTCTCTGAGTCGGGAGCGAGG
GACCAGCGCCCGGGATCGAATCCCGGACTCCCGGAGTCGAGAAAGCGCC
AAGCGGGCCCGCGCCCCTGCCGCCCTCGCCCGCCGCCGCCTCCCGCGGG
GCATTTGGACATTTTCTGCTGCGCAGCTCGCACCCCAGCTCTCGGAGCC
CGCCGCAGAGTCACACTTCGCTTGCGCGCGAGCGCTTCCCCAGCGCCTC
CGGCTCTCCTGCGCCTCGGGCTCTCCTGAGCCCCGGCGGGGCCACCGGC
CTGCTTGGCTGCGGGTTCGGGTCTGGCTGTGGGATG

TABLE 3
DNA oligonucleotides used in PCR analysis

| | | | | |
|---|---|---|---|---|
| Gene | Size (bp) | Forward Primer (5′-3′) | Reverse Primer (5′-3′) | # cycles |

Internal standards

| | | | | |
|---|---|---|---|---|
| Alpha-Tubulin | 250 | GCGTGAGTGTATCTCCATCCA | GGTAGGTGCCAGTGCGAACTT | 12-18 |
| G6PDH | 214 | GACCTGCAGAGCTCCAATCAAC | CACGACCCTCAGTACCAAAGGG | 20-24 |

Glycogen targeting Proteins

| | | | | |
|---|---|---|---|---|
| GI | 232 | CAAACTGAACAAGCCACTGAG | TCTGCTGTCGTCAGACTCAC | 20 |
| PTG | 152 | GACATTTTGTGAATAAACCGAAG | GTGGATGGCAGTGAGGGAG | 20 |

Hormones

| | | | | |
|---|---|---|---|---|
| Glucagon | 161 | ATCATTCCCAGCTTCCCAGA | CGGTTCCTCTTGGTGTTCAT | 16 |
| Insulin | 312 | TCCTGCCCCTGCTGGCCCTGC | AGTTGCAGTAGTTCTCCAG | 12 |
| Insulin2 pre-mRNA* | 190 | GGGGAGCGTGGATTCTTCTACACA | TGCCCACAGAAATGTGCCCCC | |
| Insulin2 mature mRNA* | 81 | GCACCTTTGTGGTTCTCACTTGGT | CGGGACATGGGTGTGTAGAAGAAT | |
| Somatostatin | 232 | TCTCTGCTGCCTGCGGACCT | GCCAAGAAGTACTTGGCCAGTTC | 20 |

Enzymes

| | | | | |
|---|---|---|---|---|
| G6Pase | 256 | CCTCTTCCCCATCTGGTTCC | TCACGTAGTATACACCTGCTG | 20 |
| Glucokinase | 161 | CAGATCCTGGCAGAGTTCCAG | ACTTCTGAGCCTTCTGGGGTG | 20 |
| Glut-2 | 270 | GACACCCCACTCATAGTCACAC | CAGCAATGATGAGAGCATGTG | 18 |
| Hexokinase I | 224 | TGTACGCCATGCGGCTCTC | CTCGTGGTTCACCTGCACC | 22 |
| Hexokinase II | 234 | GGAACAAACTTCCGGGTCCTGC | GTCTAGGCTGTTCTGCTGG | 22 |
| Glutamate Dehydrogenase | 258 | TGGAGGTGCTAAAGCAGGC | ATGCCTCCTTGGCTGATGG | 22 |

Transcription factors

| | | | | |
|---|---|---|---|---|
| HNF1-alpha | 236 | TCACGCCCACCAAGCAGGTC | GTTGGATGGCAGCAGGTGG | 24 |
| Islet-Brain 1 | 220 | GCTCACCGTGCACTTTAACC | GCAGGCAAACCGGTGGTC | 24 |
| NeuroD | 235 | CTTGGCCAAGAACTACATCTGG | CTGGTAGGAGTAGGGATGC | 24 |
| Nkx6.1 (rat) | 273 | GCCTGGGGTGATGCAGAG | CTTCTTTCTCCACTTGGTCC | 18 |
| Nkx6.1 (rat and hamster)* | 181 | CACCAGACCCACATTCTCC | CTCGGCTGCGTGCTTCTTT | 24 |
| Nkx6.1 (hamster) | 180 | CACGAGACCCACGTTCTCT | TCAGCCGCGTGCTTCTTC | 18 |
| Nkx2.2 | 195 | AAACTATGTTTGTGTGAGTAGC | CAGAACGTCATTTGGCAACG | 24 |
| PDX-1 | 160 | CCGTGCGCATTCCAGAGG | CTTCGTATGGGGAGATGTCC | 22 |
| Pax4 | 254 | CCAGTGTGAAGCATGCAGC | GTGGCCAGACGTGGTTTGC | 24 |
| E47/Pan-1 | 185 | GCCTGGTCAGCGGAAGC | TCTCCTCCCGCTTGATCTC | 24 |
| Brn4 | 188 | CCTCTGATGAGTTGGAACAG | GATCTTGTCAATGCTGGTCG | 24 |

Microarray confirmation genes

| | | | |
|---|---|---|---|
| BAP29* | 136 | TCGTTGAGAAGAACTCAGCCAGCA | AGCGTAACCAGACGTCTCAACACT |
| cdc25b* | 174 | TCAAAGGCGGCTACAAGGAGTTCT | AGAACTCCTTGTAGCCGCCTTTGA |
| cdkn1b(p27)* | 125 | AGCTTGCCCGAGTTCTACTACAGA | TTTGCCTGAGACCCAATTGAAGGC |
| crybb3* | 96 | TTAGAGAACTTCCAGGGCAAGCGA | TTCCACTTGGATAGAGCCCACCTT |
| Me2GlyDH* | 177 | TCGAGTCCAAGTGTTCCATGGGTT | ACGGAGACAGGTCAATCACACCAA |
| nDAP* | 192 | AGAGTTACGCCAAGGGAAGCAGAA | AAATCGGTGAGGCAGAGAAGCAGA |
| P21* | 99 | TACTTGGCTGGTGGCTCTTTGACT | ATCCAAAGCTTGGAGGCACTCTCT |
| proT-A* | 192 | TTGTGGAGGAGGCAGAGAATGGAA | TAGGAGCCTCAGCTTCCTCATCTT |
| PTPCe* | 129 | AGAATGGTCTGGGAGCAAAGGTCA | ACACACCCGGATGTTCCCATAAGT |
| Slc22a1* | 87 | AACCACGCGAGCTGTCAGGATAAT | TCAAGGCAGAGCATCTTCAGGTCA |
| ubc12* | 143 | TTAACGAGCTGAACCTGCCCAAGA | ACCCTGTCCCACCTTAAAGCTGAA |

Cyclin B1 primers

| cyclin B1 (rat and human) | 183 | ACTGGGTCGTGAAGTCACTGGAAA | AACATGGCAGTTACACCAACCAGC |
|---|---|---|---|
| cyclin B1 (rat)* | 175 | TGTGTCAGGCTTTCTCCGATGTGA | TTTCCAGTGACTTCACGACCCAGT |
| cyclin B1 (human) | 179 | GTCAGTGAACAACTGCAGGCCAAA | TTAACAGGCTCAGGTTCTGGCTCA |
| Real-time PCR probe sets | ABi Assay ID | | |
| Pdx1 | Rn00755591_m1 | | |
| Nkx6.1 | Rn00581973_m1 | | |
| Glucagon | Rn00562293_m1 | | |
| Foxo1 | Rn01769619_m1 | | |
| Pax6 | Rn00443072_m1 | | |
| GLUT2 | Rn00563565_m1 | | |
| GK | Rn00561265_ml | | |
| Cyclophilin B | Rn00574762_m1 | | |
| Ribosomal 18S | #4319413E | | |

TABLE 4
Candidate Nkx6.1 binding sites in the rat cyclin
B1 promoter.

| Location upstream of | |
|---|---|
| start codon | Promoter sequence (5′-3′) |
| 1787-1771 | TTGGCATTTTTGCCTTG |
| 1557-1541 | TTTTTATTTTCTAAGTA |
| 1439-1423 | GAAGGATTTTCCCCAGG |
| 518-502 | GGAGGATTTTGTGTGGT |
| 119-113 | GTTGTATTTTTCTTAAG |
| Nkx6.1 PBE | TTCTCATTTTGGCCCCC |

Analysis of the rat cyclin B1 promoter revealed 5 candidate Nkx6.1 binding sites. The location (relative to the start codon) and sequence of 16 nucleotide segments containing the core binding motif—ATTTT—are listed here. Additionally, the Nkx6.1 proximal binding enhancer (PBE) element from the Nkx6.1 promoter is also shown. Nucleotides of the candidate binding sites that are identical to the Nkx6.1 PBE element are underlined.

TABLE 5
Microarray analysis of Nkx6.1 silencing in 832/13 cells

| FC | Common | Genbank | Product |
|---|---|---|---|
| −4.4 | Gast | NM_012849 | gastrin |
| −4.2 | Bnip3 | NM_053420 | BCL2/adenovirus E1B 19 kDa-interacting protein 3 |
| −3.9 | Bcap29 | NP_031556 | B-cell receptor-associated protein 2 (Bap29) |
| −3.6 | Bnip3 | AF243515 | BCL2/adenovirus E1B 19 kDa-interacting protein 3 |
| −3.4 | Idh1 | NM_031510 | isocitrate dehydrogenase 1 (NADP+), soluble |
| −3.3 | Pak3 | NM_019210 | p21 (CDKN1A)-activated kinase 3 |
| −3.3 | Neurodap1 | D32249 | ring finger protein 131 |
| −3.2 | Ptma | NM_021740 | prothymosin alpha |
| −3.0 | Gucy1b3 | NM_012769 | guanylate cyclase 1, soluble, beta 3 |
| −2.9 | CHPT1 | NP_064629 | choline phosphotransferase 1 |
| −2.9 | Ptma | NM_021740 | prothymosin alpha |
| −2.6 | Pir | AW918049 | ESTs, Highly similar to PIR_MOUSE Pirin [M. musulus] |
| −2.6 | Soat1 | NM_031118 | sterol O-acyltransferase 1 |
| −2.5 | Scd2 | U67995 | stearoyl-Coenzyme A desaturase 2 |
| −2.4 | P4ha1 | X78949 | procollagen-proline, 2-oxoglutarate 4-dioxygenase |
| | | | (proline 4-hydroxylase) |
| −2.4 | Hmmr | AF133037 | hyaluronan mediated motility receptor |
| −2.4 | Nkx6a | NM_031737 | NK homeobox (Drosophila), family 6, A |
| −2.4 | Rpl4 | NM_022510 | ribosomal protein L4 |
| −2.3 | Pir | NP_081429 | pirin |
| −2.3 | Zcchc12 | AK012833 | Zinc finger CCHC domain containing protein 12(Smad- |
| | | | interacting) |
| −2.3 | Rab12 | M83676 | RAB12, member RAS oncogene family |
| −2.3 | Scd2 | NM_031841 | stearoyl-Coenzyme A desaturase 2 |
| −2.3 | Pak3 | NM_019210 | p21 (CDKN1A)-activated kinase 3 |
| −2.3 | PFDN4 | NM_002623 | ESTs, Highly similar to prefoldin 4 [Homo sapiens] |
| | | | [H. sapiens] |
| −2.2 | Cstn2 | NM34377 | calsyntenin 2 |
| −2.1 | Rpl4 | NM_022510 | ribosomal protein L4 |
| −2.1 | Cdc10 | NM_022616 | CDC10 (cell division cycle 10, S. cerevisiae, homolog) |
| −2.1 | P4ha1 | X78949 | prolyl 4-hydroxylase alpha subunit |
| −2.1 | Atp2a2 | J04022 | ATPase, Ca++ transporting, cardiac muscle, slow |
| | | | twitch 2 |
| −2.1 | Cacna2d1 | AF400662 | calcium channel, voltage-dependent, alpha2/delta |
| | | | subunit 1 |
| −2.1 | Pdcl | NM_022247 | phosducin-like |
| −2.1 | 57606 | BAA95982.1 | kiaa1458 protein (fragment) - homo sapiens |
| −2.1 | Ccnb1 | X64589 | cyclin B1 |
| −2.0 | Senp2 | NM_023989 | SUMO/sentrin specific protease 2 |
| −2.0 | Pawr | U05989 | par-4 induced by effectors of apoptosis |
| −2.0 | PFDN4 | AL133335 | prefoldin 4 (c-1) - homo sapiens |
| −2.0 | Cd47 | NM_019195 | CD47 antigen (Rh-related antigen, integrin-associated |
| | | | signal) |
| −2.0 | Itgb1 | NM_017022 | integrin beta 1 |
| 2.0 | Nbl1 | NM_031609 | neuroblastoma, suppression of tumorigenicity 1 |
| 2.0 | Serf1 | BF522362 | ESTs, Highly similar to small EDRK-rich factor 1 |
| 2.0 | Hpgd | NM_024390 | NAD-dependent 15-hydroxyprostaglandin |
| | | | dehydrogenase |
| 2.0 | Fabp5 | U13253 | fatty acid binding protein 5, epidermal |
| 2.0 | Nme3 | NM_053507 | non-metastatic cells 3, protein expressed in |
| 2.0 | Slfn4 | Y17327 | CDK107 |
| 2.0 | Txnip | NP_076208 | similar to thioredoxin interacting factor - mus musculus |
| 2.1 | Slc25a10 | AJ223355 | solute carrier family 25 (mitochondrial carrier; |
| | | | dicarboxylate transporter), member 10 |
| 2.1 | Vsp45a | U81160 | vesicular transport protein rvps45 |
| 2.1 | Ggtp | M33821 | gamma-glutamyl transpeptidase |
| 2.1 | Sh3kbp1 | AF255888 | SH3 domain-containing adapter protein |
| 2.1 | Vsp45a | U81160 | vacuolar protein sorting 45 |
| 2.1 | Zfp106 | NP_035873 | SH3-domain binding protein 3 - zinc finger protein 106 - |
| | | | mus musculus |
| 2.1 | Bk | U30831 | B/K protein |
| 2.1 | Pde9a | NM38543 | phosphodiesterase 9A |
| 2.1 | C1galt1 | NM_022950 | core1 UDP-galactose:N-acetylgalactosamine-alpha-R |
| | | | beta 1,3-galactosyltransferase |
| 2.1 | Asah | NM_053407 | N-acylsphingosine amidohydrolase 1 |
| 2.1 | MrpI36 | BC009166 | mitochondrial ribosomal protein I36 - mus musculus |
| 2.1 | S100a4 | NM_012618 | S100 calcium-binding protein A4 |
| 2.1 | Acac | X53003 | acetyl-coenzyme A carboxylase |
| 2.2 | Acac | NM_022193 | acetyl-coenzyme A carboxylase |
| 2.2 | Bdnf | NM_012513 | brain derived neurotrophic factor |
| 2.2 | Lin7c | NM_021851 | lin-7 homolog C |
| 2.2 | Asah | AF214647 | N-acylsphingosine amidohydrolase (acid ceramidase) |
| 2.2 | Atp5f1 | M35052 | ATP synthase, H+ transporting, mitochondrial F0 |
| | | | complex, subunit b, isoform 1 |
| 2.2 | Gsto1 | AB008807 | glutathione S-transferase omega 1 |
| 2.2 | Ift57 | AF274590 | huntingtin-interacting protein-1 protein interactor - mus |
| | | | musculus |
| 2.2 | Jundp2 | NM_053894 | Jun dimerization protein 2 |
| 2.3 | 75564 | AK006440 | Protein C6orf206 homolog - mus musculus |
| 2.3 | Hsbp1 | AF522937 | heat shock factor binding protein 1 |
| 2.3 | Hsbp1 | BC002153 | similar to heat shock factor binding protein 1 - mus |
| | | | musculus |
| 2.3 | Slc12a4 | NM_019229 | solute carrier family 12, member 4 |
| 2.4 | Sh3kbp1 | AF255884 | SH3 domain-containing adapter protein |
| 2.4 | Nelf | NM_057190 | nasal embryonic LHRH factor |
| 2.4 | Cyp4f14 | NM_019623 | cytochrome P450 4F1 |
| 2.4 | Atp6v0e2 | NP_598525 | similar to ATPase, H+ transporting, V0 subunit - mus |
| | | | musculus |
| 2.4 | Lklf | AF181251 | ESTs, Moderately similar to Kruppel-like factor 6 |
| 2.4 | Vdup1 | U30789 | upregulated by 1,25-dihydroxyvitamin D-3 |
| 2.4 | S100a4 | NM_012618 | S100 calcium-binding protein A4 |
| 2.4 | Hsbp1 | BC002153 | similar to heat shock factor binding protein 1 - mus |
| | | | musculus |
| 2.4 | AF146738 | AF146738 | testis specific protein |
| 2.4 | Ache | S50879 | Acetylcholinesterase |
| 2.4 | Lcat | NM_017024 | lecithin cholesterol acyltransferase |
| 2.4 | Guca2b | NM_022284 | guanylate cyclase activator 2b |
| 2.4 | S100a3 | AF140231 | S100 calcium binding protein A3 |
| 2.5 | BRD1 | AW525127 | Highly similar to bromodomain containing protein 1; |
| | | | BR140-like |
| 2.5 | Cspg5 | NM_019284 | chondroitin sulfate proteoglycan 5 |
| 2.5 | AF146738 | AF146738 | testis specific protein |
| 2.5 | Ocm; OM | X15836 | Oncomodulin |
| 2.5 | LOC94168 | U19485 | spp-24 precursor |
| 2.5 | Pcp4 | NM_013002 | Purkinje cell protein 4 |
| 2.5 | Ldhb | NM_012595 | lactate dehydrogenase B |
| 2.6 | Camk4 | NM_012727 | calcium/calmodulin-dependent protein kinase IV |
| 2.6 | Matk | NM_021859 | non-receptor protein kinase protein |
| 2.7 | Fgb | U05675 | fibrinogen, beta polypeptide |
| 2.7 | Slc2a4 | NM_012751 | solute carrier family 2, member 4 |
| 2.7 | Klf2; Lklf | AF181251 | Kruppel-like factor |
| 2.7 | Inexa | NM_019128 | internexin, alpha |
| 2.8 | Nelf | AJ293697 | nasal embryonic LHRH factor |
| 2.8 | S100a3 | NM_053681 | S100 calcium binding protein A3 |
| 2.8 | Matk | NM_021859 | non-receptor protein kinase protein |
| 2.9 | Gipc2 | NP_058563 | similar to semaf cytoplasmic domain associated protein 2 |
| 2.9 | Rgs19 | AF068136 | ESTs, Moderately similar to regulator of G-protein |
| | | | signaling 19 |
| 2.9 | Fgb | M27220 | fibrinogen, beta polypeptide |
| 2.9 | Bv8 | NM38852 | homolog of mouse Bv8; prokineticin 2 precursor |
| 2.9 | Prkaa2 | NM_023991 | AMP-activated protein kinase alpha 2 catalytic subunit |
| 3.0 | Tac1 | NM_012666 | tachykinin 1 |
| 3.1 | Ldhb | NM_012595 | lactate dehydrogenase B |
| 3.3 | Crybb3 | NM_031690 | crystallin, beta B3 |
| 3.3 | Crybb3 | NM_031690 | crystallin, beta B3 |
| 3.8 | Ube2m | NP_663553 | similar to ubiquitin-conjugating enzyme e2m (ubc12 |
| | | | homolog) |
| 3.8 | Dmgdh | X55995 | dimethylglycine dehydrogenase precursor |
| 3.9 | Ptpre | Y07834 | protein tyrosine phosphatase epsilon-like 2 |
| 4.1 | Ube2m | NP_663553 | similar to ubiquitin-conjugating enzyme e2m (ubc12 |
| | | | homolog) |
| 4.6 | Ptpre | D78610 | protein tyrosine phosphatase epsilon C |
| 5.2 | Slc22a1 | NM_012697 | solute carrier family 22 (organic cation transporter), |
| | | | member 1 |

Fold change (FC) represents the relative level of expression from samples treated with Ad-siNkx6.1 versus Ad-siRNAcontrol. Therefore, a negative value or positive value indicates that the relative expression of that gene was lower (−FC) or higher (+FC), respectively, in cells treated with Ad-siNkx6.1 compared to cells treated with Ad-siRNAcontrol. Also provided are the common gene name, GenBank identifier, and a description of the gene product.

TABLE 6
Nkx6.1 overexpression microarray results
List of Nkx6.1 overexpression microarray results

| Fold | | | |
|---|---|---|---|
| Change | Common | Genbank | Description |
| 9.97 | | — | — |
| 6.69 | Pttg1 | — | SECURIN (PITUITARY TUMOR-TRANSFORMING |
| | | | PROTEIN 1). [Source: SWISSPROT; Acc: P97613] |
| 6.00 | Cdca3 | BC079204 | Cell division cycle associated 3 |
| 5.83 | Cdc2a | — | CELL DIVISION CONTROL PROTEIN 2 HOMOLOG (EC |
| | | | 2.7.1.—) (P34 PROTEIN KINASE) (CYCLIN-DEPENDENT |
| | | | KINASE 1) (CDK1). [Source: SWISSPROT; Acc: P39951] |
| 5.47 | na | — | — |
| 4.84 | Cidea_predicted | — | — |
| 4.55 | Loc192264 | D88586, | EOSINOPHIL CATIONIC PROTEIN PRECURSOR (EC |
| | | D88586, | 3.1.27.—) (ECP) (RIBONUCLEASE 3) (RNASE 3). |
| | | CA507907 | [Source: SWISSPROT; Acc: P70709] |
| 4.49 | | — | G2/MITOTIC-SPECIFIC CYCLIN B1. |
| | | | [Source: SWISSPROT; Acc: P30277] |
| 4.23 | Ube2c_predicted | — | — |
| 4.20 | Vgf | M60525 | VGF nerve growth factor inducible |
| 4.16 | Ccna2 | AY623028 | Cyclin A2 |
| 4.09 | na | — | — |
| 4.08 | Pbk_predicted | — | — |
| 4.07 | Icam1 | BC081837 | Intercellular adhesion molecule 1 |
| 4.05 | Avp | — | VASOPRESSIN-NEUROPHYSIN 2-COPEPTIN |
| | | | PRECURSOR (AVP-NPII) [CONTAINS: ARG- |
| | | | VASOPRESSIN; NEUROPHYSIN 2 (NEUROPHYSIN-I); |
| | | | COPEPTIN]. [Source: SWISSPROT; Acc: P01186] |
| 3.84 | | M30689, | LY6-B ANTIGEN GENE. |
| | | M30689 | [Source: RefSeq; Acc: NM_139257] |
| 3.71 | Slc39a4_predicted | — | — |
| 3.71 | Dlg7_predicted | — | — |
| 3.59 | Cdc20 | BC085691 | Cell division cycle 20 homolog (S. cerevisiae) |
| 3.59 | Nr4a3 | D38530 | Nuclear receptor subfamily 4, group A, member 3 |
| 3.56 | Stk6 | BC086984 | Serine/threonine kinase 6 |
| 3.54 | Cenpe_predicted | — | — |
| 3.51 | Hmmr | AF133037, AF133037 | HYALURONAN MEDIATED MOTILITY RECEPTOR |
| | | | (INTRACELLULAR HYALURONIC ACID BINDING |
| | | | PROTEIN) (RECEPTOR FOR HYALURONAN- |
| | | | MEDIATED MOTILITY). |
| | | | [Source: SWISSPROT; Acc: P97779] |
| 3.45 | Loc192264 | D88586, | EOSINOPHIL CATIONIC PROTEIN PRECURSOR (EC |
| | | D88586, | 3.1.27.—) (ECP) (RIBONUCLEASE 3) (RNASE 3). |
| | | CA507907 | [Source: SWISSPROT; Acc: P70709] |
| 3.44 | Fanca_predicted | — | — |
| 3.41 | Mki67_predicted | — | — |
| 3.40 | Ns5atp9 | — | — |
| 3.38 | Cdkn3_predicted | — | — |
| 3.37 | Mphosph1_predicted | — | — |
| 3.35 | Mastl_predicted | — | — |
| 3.33 | Grin2c | U08259 | Glutamate receptor, ionotropic, NMDA2C |
| 3.31 | | — | — |
| 3.29 | | — | STATHMIN (PHOSPHOPROTEIN P19) (PP19) |
| | | | (ONCOPROTEIN 18) (OP18) (LEUKEMIA-ASSOCIATED |
| | | | PHOSPHOPROTEIN P18) (PP17) (PROSOLIN) |
| | | | (METABLASTIN) (PR22 PROTEIN). |
| | | | [Source: SWISSPROT; Acc: P13668] |
| 3.23 | | — | — |
| 3.18 | Cxcl2 | — | MACROPHAGE INFLAMMATORY PROTEIN 2 |
| | | | PRECURSOR (MIP2) (CINC-3). |
| | | | [Source: SWISSPROT; Acc: P30348] |
| 3.15 | Prc1_predicted | — | — |
| 3.14 | Cdca1_predicted | BC078993 | Cell division cycle associated 1 (predicted) |
| 3.10 | Kifc1 | BC083827 | Kinesin family member C1 |
| 3.10 | Dll3 | AF084576, AF084576 | DELTA-LIKE PROTEIN 3 PRECURSOR (DROSOPHILA |
| | | | DELTA HOMOLOG 3). |
| | | | [Source: SWISSPROT; Acc: O88671] |
| 3.09 | | — | MATRIX GLA-PROTEIN PRECURSOR (MGP). |
| | | | [Source: SWISSPROT; Acc: P08494] |
| 3.07 | Blvrb_predicted | — | — |
| 3.06 | Irf7_predicted | — | — |
| 3.06 | Ckap2_predicted | — | — |
| 3.04 | | — | — |
| 2.99 | Psmb9 | — | PROTEASOME SUBUNIT BETA TYPE 9 PRECURSOR |
| | | | (EC 3.4.25.1) (PROTEASOME CHAIN 7) (MACROPAIN |
| | | | CHAIN 7) (MULTICATALYTIC ENDOPEPTIDASE |
| | | | COMPLEX CHAIN 7) (RING12 PROTEIN) (LOW |
| | | | MOLECULAR MASS PROTEIN 2). |
| | | | [Source: SWISSPROT; Acc: P28077] |
| 2.98 | Exo1_predicted | — | — |
| 2.97 | Slpi | — | SECRETORY LEUKOCYTE PROTEASE INHIBITOR. |
| | | | [Source: RefSeq; Acc: NM_053372] |
| 2.93 | Hmgb2 | BC089854 | High mobility group box 2 |
| 2.93 | PVR | L12025 | Poliovirus receptor |
| 2.91 | | — | G1/S-SPECIFIC CYCLIN E1. |
| | | | [Source: SWISSPROT; Acc: P39949] |
| 2.89 | na | — | — |
| 2.88 | na | — | — |
| 2.87 | Tpx2_predicted | — | — |
| 2.81 | | — | — |
| 2.80 | Bzrp | — | PERIPHERAL-TYPE BENZODIAZEPINE RECEPTOR |
| | | | (PBR) (PKBS) (MITOCHONDRIAL BENZODIAZEPINE |
| | | | RECEPTOR). [Source: SWISSPROT; Acc: P16257] |
| 2.80 | Rs21c6 | — | RS21-C6 PROTEIN. [Source: RefSeq; Acc: NM_138892] |
| 2.79 | Melk_predicted | — | — |
| 2.79 | | — | — |
| 2.79 | Nol3 | U40627 | Nucleolar protein 3 (apoptosis repressor with CARD |
| | | | domain) |
| 2.76 | Cdca8 | BC079274 | Similar to embryonic stem cell-related |
| 2.75 | Il8ra | — | HIGH AFFINITY INTERLEUKIN-8 RECEPTOR A (IL-8R |
| | | | A) (CXCR-1). [Source: SWISSPROT; Acc: P70612] |
| 2.74 | Biklk | AF372501, AF372501 | BCL2-INTERACTING KILLER-LIKE. |
| | | | [Source: RefSeq; Acc: NM_053704] |
| 2.73 | Cpm_predicted | — | — |
| 2.72 | Tacc3 | — | — |
| 2.68 | na | CF115007 | Similar to Putative protein C21orf45 (predicted) |
| 2.66 | Nrm | — | — |
| 2.60 | Akr7a3 | BC078872 | Aldo-keto reductase family 7, member A3 (aflatoxin |
| | | | aldehyde reductase) |
| 2.59 | Pap | — | PANCREATITIS-ASSOCIATED PROTEIN 1 |
| | | | PRECURSOR (PEPTIDE 23) (REG-2). |
| | | | [Source: SWISSPROT; Acc: P25031] |
| 2.55 | | X57523, | ANTIGEN PEPTIDE TRANSPORTER 1 (APT1). |
| | | X57523 | [Source: SWISSPROT; Acc: P36370] |
| 2.52 | na | — | — |
| 2.50 | | — | CYTOKERATIN-18 (FRAGMENT). |
| | | | [Source: SPTREMBL; Acc: P97598] |
| 2.50 | Dlg7_predicted | — | — |
| 2.50 | Cxcl11 | AF537209 | Chemokine (C—X—C motif) ligand 11 |
| 2.46 | RT1-A3 | BC088465, X82669, | CLASS I MHC HEAVY CHAIN RT1.A(N) ANTIGEN |
| | | \X82669 | PRECURSOR. [Source: SPTREMBL; Acc: P79588] |
| 2.46 | Brca1 | AF036760 | Hypothetical gene supported by NM_012514 |
| 2.46 | Kif20a_predicted | — | — |
| 2.45 | Gadd45b | BC085337 | Growth arrest and DNA-damage-inducible 45 beta |
| | | | (predicted) |
| 2.45 | | — | NEURAL F BOX PROTEIN NFB42. |
| | | | [Source: RefSeq; Acc: NM_053511] |
| 2.45 | | — | — |
| 2.41 | | — | — |
| 2.41 | | — | CARBONIC ANHYDRASE-RELATED XI PROTEIN. |
| | | | [Source: RefSeq; Acc: NM_175708] |
| 2.41 | | — | CYSTEINE-RICH PROTEIN 1 (CYSTEINE-RICH |
| | | | INTESTINAL PROTEIN) (CRIP). |
| | | | [Source: SWISSPROT; Acc: P04006] |
| 2.40 | | AA94459 | — |
| | | BC089854 | |
| 2.36 | Cdc6_predicted | — | — |
| 2.35 | na | — | — |
| 2.34 | Gfra2 | U97143 | Glial cell line derived neurotrophic factor family receptor |
| | | | alpha 2 |
| 2.33 | na | — | — |
| 2.32 | | — | — |
| 2.32 | Slc5a3 | AY231162 | Solute carrier family 5 (inositol transporters), member 3 |
| 2.32 | | — | PUTATIVE RNA BINDING PROTEIN (FRAGMENT). |
| | | | [Source: SPTREMBL; Acc: O35978] |
| 2.31 | Gapd | BC059110 | Similar to glyceraldehyde-3-phosphate dehydrogenase |
| 2.30 | Dnmt3l | — | — |
| 2.30 | Mmp14 | X91785 | Matrix metalloproteinase 14 (membrane-inserted) |
| 2.28 | | — | PUTATIVE RNA BINDING PROTEIN 1 (FRAGMENT). |
| | | | [Source: SPTREMBL; Acc: O08882] |
| 2.27 | Cxcl10 | BC058444 | Chemokine (C—X—C motif) ligand 10 |
| 2.26 | Fshprh1 | X90355 | Follicle stimulating hormone primary response gene 1 |
| 2.26 | Prkwnk4 | — | PROTEIN KINASE, LYSINE DEFICIENT 4; WNK4 |
| | | | SER/THR KINASE. [Source: RefSeq; Acc: NM_175579] |
| 2.24 | Gpx2 | — | — |
| 2.23 | Mrc1_predicted | CF110122 | Mannose receptor, C type 1 (predicted) |
| 2.23 | Cyln2 | AJ000485 | Cytoplasmic linker 2 |
| 2.22 | | — | — |
| 2.21 | Pold2_predicted | BC079267 | Polymerase (DNA directed), delta 2, regulatory subunit |
| | | | (predicted) |
| 2.21 | Pfkfb1 | J04197 | 6-phosphofructo-2-kinase/fructose-2,6-biphosphatase 1 |
| 2.21 | | — | — |
| 2.20 | Gmnn_predicted | — | — |
| 2.19 | na | — | — |
| 2.19 | Dusp1 | X84004, | DUAL SPECIFICITY PROTEIN PHOSPHATASE 1 (EC |
| | | X84004 | 3.1.3.48) (EC 3.1.3.16) (MAP KINASE PHOSPHATASE-1) |
| | | | (MKP-1) (PROTEIN-TYROSINE PHOSPHATASE CL100). |
| | | | [Source: SWISSPROT; Acc: Q64623] |
| 2.18 | Cstb | — | CYSTATIN B (LIVER THIOL PROTEINASE INHIBITOR) |
| | | | (STEFIN B) (CYSTATIN BETA). |
| | | | [Source: SWISSPROT; Acc: P01041] |
| 2.18 | Id1 | — | DNA-BINDING PROTEIN INHIBITOR ID-1. |
| | | | [Source: SWISSPROT; Acc: P41135] |
| 2.17 | | — | — |
| 2.16 | Pspla1 | BC078727 | Phosphatidylserine-specific phospholipase A1 |
| 2.16 | na | — | — |
| 2.14 | Gmfg | — | GLIA MATURATION FACTOR BETA (GMF-BETA). |
| | | | [Source: SWISSPROT; Acc: Q63228] |
| 2.14 | MGC108931 | BC088212 | Cyclin B2 (predicted) |
| 2.13 | Slc25a10 | BC081734 | Solute carrier family 25 (mitochondrial carrier; |
| | | | dicarboxylate transporter), member 10 |
| 2.13 | Ccne2_predicted | — | — |
| 2.13 | na | — | — |
| 2.12 | na | — | — |
| 2.12 | | AY53992 | — |
| | | AY539877 | |
| 2.11 | Kif22 | BC088421 | Kinesin family member 22 (predicted) |
| 2.11 | Rhod_predicted | — | — |
| 2.09 | Myl3 | — | MYOSIN LIGHT CHAIN 1, SLOW-TWITCH MUSCLE |
| | | | B/VENTRICULAR ISOFORM. |
| | | | [Source: SWISSPROT; Acc: P16409] |
| 2.09 | na | — | — |
| 2.08 | | — | — |
| 2.08 | Mll5_predicted | — | — |
| 2.07 | Cdc42ep5_predicted | — | — |
| 2.07 | Kif11 | — | KINESIN-RELATED PROTEIN KRP6 (FRAGMENT). |
| | | | [Source: SPTREMBL; Acc: O54723] |
| 2.07 | Cxcl12 | AF217564 | Chemokine (C—X—C motif) ligand 12 |
| 2.07 | Myadm | AY344060 | Myeloid-associated differentiation marker |
| 2.06 | Bub1_predicted | — | — |
| 2.06 | na | — | — |
| 2.06 | Mx2 | — | INTERFERON-INDUCED GTP-BINDING PROTEIN MX2. |
| | | | [Source: SWISSPROT; Acc: P18589] |
| 2.04 | na | — | — |
| 2.04 | Padi2 | J05022 | Peptidyl arginine deiminase, type II |
| 2.04 | Hsd11b1 | AY387051 | Hydroxysteroid 11-beta dehydrogenase 1 |
| 2.04 | Rab13 | — | RAS-RELATED PROTEIN RAB-13 (FRAGMENT). |
| | | | [Source: SWISSPROT; Acc: P35286] |
| 2.04 | | — | — |
| 2.03 | Pde4b | U95748, | — |
| | | U95748 | |
| 2.03 | | — | GALACTOSE-1-PHOSPHATE |
| | | | URIDYLYLTRANSFERASE (EC 2.7.7.12) (GAL-1-P |
| | | | URIDYLYLTRANSFERASE) (UDP-GLUCOSE--HEXOSE- |
| | | | 1-PHOSPHATE URIDYLYLTRANSFERASE). |
| | | | [Source: SWISSPROT; Acc: P43424] |
| 2.03 | | — | GLUTATHIONE S-TRANSFERASE YC-1 (EC 2.5.1.18) |
| | | | (CHAIN 2) (GST YC1) (GST CLASS-ALPHA). |
| | | | [Source: SWISSPROT; Acc: P04904] |
| 2.03 | | — | — |
| 2.02 | na | — | — |
| 2.02 | Adora1 | Y12519 | Adenosine A1 receptor |
| 2.01 | na | — | — |
| 2.01 | Mad2l1_predicted | — | — |
| 2.00 | Ly6c | M30691 | Ly6-C antigen |
| 0.50 | Tox_predicted | — | — |
| 0.50 | | — | — |
| 0.50 | Zfp131_predicted | — | — |
| 0.50 | Snx25_predicted | BC086510 | Sorting nexin 25 (predicted) |
| 0.50 | na | — | — |
| 0.50 | | — | — |
| 0.50 | | BC083854 | CDNA clone IMAGE: 7124415 |
| 0.49 | na | — | — |
| 0.49 | na | — | — |
| 0.49 | | — | — |
| 0.49 | na | AA955567 | Similar to Spindlin-like |
| 0.49 | | — | — |
| 0.49 | Fanca_predicted | — | — |
| 0.49 | Snrpn | M29293 | Small nuclear ribonucleoprotein N |
| 0.49 | | — | — |
| 0.48 | Nfyb | BC089791 | Nuclear transcription factor-Y beta |
| 0.48 | | — | — |
| 0.48 | MGC94588 | BC085859 | Eukaryotic translation initiation factor 4A2 (predicted) |
| 0.48 | LOC306198 | BC088243 | Citrate lyase beta like (predicted) |
| 0.48 | na | — | — |
| 0.48 | Sytl5 | AB098162 | Synaptotagmin-like 5 |
| 0.48 | Pklr | M11709 | Pyruvate kinase, liver and RBC |
| 0.47 | MGC94736 | BC083777 | Similar to hypothetical protein MGC35097 |
| 0.47 | na | — | — |
| 0.47 | | — | — |
| 0.47 | na | — | — |
| 0.47 | | — | — |
| 0.47 | | — | — |
| 0.47 | Star | AF044081 | Steroidogenic acute regulatory protein |
| 0.46 | Epb4.1l3 | AB032827 | ERYTHROCYTE PROTEIN BAND 4.1-LIKE 3. |
| | | AB032827 | [Source: RefSeq; Acc: NM_053927] |
| 0.46 | Adora2a | BC081727 | Adenosine A2a receptor |
| 0.46 | Gnao | M17526 | Guanine nucleotide binding protein, alpha o |
| 0.46 | Vcip135 | AF289091 | Valosin-containing protein (p97)/p47 complex-interacting |
| | | | protein p135 |
| 0.46 | | CF110087 | Small nuclear RNA activating complex, polypeptide 3 |
| | | | (predicted) |
| 0.46 | LOC361335 | BC085946 | Similar to A630042L21Rik protein |
| 0.46 | Hexa | BC082097 | Hexosaminidase A |
| 0.46 | Ptk2 | AF020777 | PTK2 protein tyrosine kinase 2 |
| 0.46 | | CB313014 | Transcribed locus |
| 0.46 | | — | — |
| 0.46 | na | — | — |
| 0.46 | na | — | — |
| 0.46 | Flnb_predicted | — | — |
| 0.46 | | — | — |
| 0.45 | Iqcb1_predicted | — | — |
| 0.45 | | — | — |
| 0.45 | MGC93874 | BC083571 | Progesterone receptor membrane component 2 |
| | | | (predicted) |
| 0.45 | C2 | AY325253BC070923 | — |
| 0.45 | Sema3e_predicted | — | — |
| 0.45 | Ocil | — | — |
| 0.45 | Sqle | D37920 | Squalene epoxidase |
| 0.45 | na | — | — |
| 0.45 | Ddx25 | AF142629 | DEAD (Asp-Glu-Ala-Asp) box polypeptide 25 |
| 0.45 | | — | — |
| 0.44 | na | — | — |
| 0.44 | | — | — |
| 0.44 | | — | — |
| 0.44 | Mettl3_predicted | — | — |
| 0.44 | Arhgap24_predicted; | AB108670 | Rho GTPase activating protein 24 (predicted) |
| | DR-NR#2 | | |
| 0.44 | Pcyt1b | AF197561 | Phosphorylase kinase alpha 1 |
| 0.44 | Usp25_predicted | — | — |
| 0.44 | na | BC087711 | Similar to KIAA1900 protein (predicted) |
| 0.44 | | — | — |
| 0.43 | Galnt14_predicted | BC079128 | UDP-N-acetyl-alpha-D-galactosamine:polypeptide N- |
| | | | acetylgalactosaminyltransferase 14 (predicted) |
| 0.43 | Mtmr4_predicted | — | — |
| 0.43 | Kcnh6 | AF016192, AF016192 | POTASSIUM VOLTAGE-GATED CHANNEL SUBFAMILY |
| | | | H MEMBER 6 (ETHER-A-GO-GO RELATED GENE |
| | | | POTASSIUM CHANNEL 2) (ETHER-A-GO-GO RELATED |
| | | | PROTEIN 2) (EAG RELATED PROTEIN 2). |
| | | | [Source: SWISSPROT; Acc: O54853] |
| 0.43 | LOC498229 | BC079284 | Similar to RIKEN cDNA 4930429O20 |
| 0.42 | Ica1 | L20900 | Islet cell autoantigen 1 |
| 0.42 | Idh1 | L35317 | Isocitrate dehydrogenase 1 (NADP+), soluble |
| 0.42 | Pax6 | U69644 | Paired box gene 6 |
| 0.42 | | CB313014 | Transcribed locus |
| 0.42 | MGC94686 | BC083753 | Similar to RIKEN cDNA 2810413N20 |
| 0.42 | na | — | — |
| 0.42 | MGC105536 | BC086397 | Aldo-keto reductase family 1, member E1 (predicted) |
| 0.42 | | — | — |
| 0.42 | na | — | — |
| 0.42 | | — | — |
| 0.41 | LOC497934 | BC082026 | Similar to hypothetical protein FLJ20014 |
| 0.41 | Enpp2 | D28560, | ECTONUCLEOTIDE |
| | | D28560 | PYROPHOSPHATASE/PHOSPHODIESTERASE 2 (E- |
| | | | NPP 2) (PHOSPHODIESTERASE I/NUCLEOTIDE |
| | | | PYROPHOSPHATASE 2) (PHOSPHODIESTERASE I |
| | | | ALPHA) (PD-IALPHA) [INCLUDES: ALKALINE |
| | | | PHOSPHODIESTERASE I (EC 3.1.4.1); NUCLEOTIDE |
| | | | PYROPHOSPHATASE (EC 3.6.1.9) (NPPASE)]. |
| | | | [Source: SWISSPROT; Acc: Q64610] |
| 0.41 | Fhit | — | — |
| 0.41 | Metap2 | L10652 | Methionine aminopeptidase 2 |
| 0.41 | OSP94 | — | — |
| 0.41 | MGC105536 | BC086397 | Aldo-keto reductase family 1, member E1 (predicted) |
| 0.41 | Agt | BC078741 | Angiotensinogen |
| 0.41 | na | — | ELAV-LIKE PROTEIN 4 (PARANEOPLASTIC |
| | | | ENCEPHALOMYELITIS ANTIGEN HUD) (HU-ANTIGEN |
| | | | D). [Source: SWISSPROT; Acc: O09032] |
| 0.41 | | — | — |
| 0.40 | na | — | — |
| 0.40 | na | — | — |
| 0.40 | Lhfpl5 | BC087673 | Lipoma HMGIC fusion partner-like 5 |
| 0.40 | Usp11 | — | — |
| 0.40 | Serpina6 | BC088300 | Serine (or cysteine) proteinase inhibitor, clade A (alpha-1 |
| | | | antiproteinase, antitrypsin), member 6 |
| 0.40 | Rbbp7 | AF090306 | Retinoblastoma binding protein 7 |
| 0.40 | Kctd13 | AY305029 | Potassium channel tetramerisation domain containing 13 |
| 0.39 | Ehhadh | BC089777 | Enoyl-Coenzyme A, hydratase/3-hydroxyacyl Coenzyme |
| | | | A dehydrogenase |
| 0.39 | Cntn3 | U11031 | Hypothetical gene supported by NM_019329 |
| 0.39 | Proc | X64336 | Protein C |
| 0.39 | Dcps | BC089773 | MRNA decapping enzyme |
| 0.39 | Satb1_predicted | BC085814 | Special AT-rich sequence binding protein 1 (predicted) |
| 0.39 | | — | NEURONAL PENTRAXIN II PRECURSOR (NP-II) (NP2) |
| | | | (NEURONAL ACTIVITY-REGULATED PENTRAXIN). |
| | | | [Source: SWISSPROT; Acc: P97738] |
| 0.39 | na | — | — |
| 0.38 | Epb4.1l4b_predicted | — | — |
| 0.38 | Cntnap4_predicted | — | — |
| 0.38 | RSB-11-77 | AY149342 | RSB-11-77 protein |
| 0.38 | Gad2 | M72422 | Glutamate decarboxylase 2 |
| 0.38 | Ca2 | CF111017 | Carbonic anhydrase 2 |
| 0.38 | Casp2 | U77933, | CASPASE-2 PRECURSOR (EC 3.4.22.—) (CASP-2) (ICH- |
| | | U77933 | 1 PROTEASE) (FRAGMENT). |
| | | | [Source: SWISSPROT; Acc: P55215] |
| 0.38 | Pigh_predicted | AA999188 | Phosphatidylinositol glycan, class H (predicted) |
| 0.38 | | — | — |
| 0.37 | Prkcb1 | X04440 | Protein kinase C, beta 1 |
| 0.37 | Fez1 | BC087740 | Fasciculation and elongation protein zeta 1 (zygin I) |
| 0.37 | Vps39_predicted | BC087048 | Vacuolar protein sorting 39 (yeast) (predicted) |
| 0.37 | Kcnn2 | U69882 | Potassium intermediate/small conductance calcium- |
| | | | activated channel, subfamily N, member 2 |
| 0.37 | MGC94368 | BC083610 | Similar to hypothetical protein MGC50721 |
| 0.37 | | — | — |
| 0.36 | na | — | — |
| 0.36 | MGC105536 | BC086397 | Aldo-keto reductase family 1, member E1 (predicted) |
| 0.36 | LOC313436 | BC089115 | Similar to RIKEN cDNA 2810028A01 |
| 0.36 | Cd53 | M57276 | CD53 antigen |
| 0.36 | na | — | — |
| 0.36 | Gnat3 | X65747, | — |
| | | X65747 | |
| 0.36 | Igfbp1 | BC078889 | Insulin-like growth factor binding protein 1 |
| 0.36 | na | — | — |
| 0.36 | Itpr1 | J05510, | INOSITOL 1,4,5-TRISPHOSPHATE RECEPTOR TYPE 1 |
| | | J05510 | (TYPE 1 INOSITOL 1,4,5-TRISPHOSPHATE |
| | | | RECEPTOR) (TYPE 1 INSP3 RECEPTOR) (IP3 |
| | | | RECEPTOR ISOFORM 1) (INSP3R1) (IP-3-R). |
| | | | [Source: SWISSPROT; Acc: P29994] |
| 0.36 | Itih1_predicted | — | — |
| 0.36 | na | — | — |
| 0.35 | Cyp2r1_predicted | — | — |
| 0.35 | | — | — |
| 0.35 | | — | — |
| 0.34 | Stau2 | — | — |
| 0.34 | Igfbp2 | J04486 | Insulin-like growth factor binding protein 2 |
| 0.34 | | — | — |
| 0.34 | na | — | — |
| 0.33 | Gls | M65150 | Glutaminase |
| 0.33 | Ros1 | M35105 | Transforming growth factor beta regulated gene 1 |
| | | | (predicted) |
| 0.33 | Taf6l_predicted | — | — |
| 0.33 | Gls | M65150 | Glutaminase |
| 0.33 | | — | — |
| 0.32 | Apob | AY318958 | Apolipoprotein B |
| 0.32 | Hgfac | BC089871 | Hepatocyte growth factor activator |
| 0.30 | Kcnc2 | M34052, | POTASSIUM VOLTAGE-GATED CHANNEL SUBFAMILY |
| | | M34052 | C MEMBER 2 (POTASSIUM CHANNEL KV3.2) (KSHIIIA). |
| | | | [Source: SWISSPROT; Acc: P22462] |
| 0.30 | Mcoln3_predicted | BC079200 | Mucolipin 3 (predicted) |
| 0.30 | | — | — |
| 0.29 | | — | — |
| 0.29 | | — | — |
| 0.29 | Zfp110_predicted | — | ZINC FINGER PROTEIN 13 (FRAGMENT). |
| | | | [Source: SPTREMBL; Acc: P97669] |
| 0.29 | RT1-Da | — | INTEGRAL MEMBRANE PROTEIN ALPHA PRECHAIN |
| | | | PRECURSOR. [Source: SPTREMBL; Acc: Q95572] |
| 0.29 | Cpa6_predicted | — | — |
| 0.28 | pips | AB051807 | G protein-coupled receptor associated sorting protein 1 |
| 0.28 | Ms4a7_predicted | — | — |
| 0.28 | Sstr2 | M96817 | Somatostatin receptor 2 |
| 0.28 | Kcnj13 | AB013890 | INWARD RECTIFIER POTASSIUM CHANNEL 13 |
| | | AB013890 | (POTASSIUM CHANNEL, INWARDLY RECTIFYING, |
| | | | SUBFAMILY J, MEMBER 13) (INWARD RECTIFIER K+ |
| | | | CHANNEL KIR7.1). [Source: SWISSPROT; Acc: O70617] |
| 0.27 | Hhex | D86383 | Hematopoietically expressed homeobox |
| 0.26 | Lgals2 | — | GALECTIN-2. [Source: SWISSPROT; Acc: Q9Z144] |
| 0.26 | na | — | — |
| 0.26 | | U27322, | VASOPRESSIN V1B RECEPTOR (V1BR) (AVPR V1B) |
| | | U27322 | (VASOPRESSIN V3 RECEPTOR) (AVPR V3) |
| | | | (ANTIDIURETIC HORMONE RECEPTOR 1B). |
| | | | [Source: SWISSPROT; Acc: P48974] |
| 0.26 | MGC108825 | BC088180 | Apolipoprotein H (predicted) |
| 0.24 | na | — | — |
| 0.23 | | — | — |
| 0.22 | | CB705662 | Transcribed locus, strongly similar to NP_766294.1 |
| | | | RIKEN cDNA 9330182L06 [Mus musculus] |
| 0.21 | Lrrn3 | AF291437 | Leucine rich repeat protein 3, neuronal |
| 0.20 | Ptger2 | U94708 | Prostaglandin E receptor 2, subtype EP2 |
| 0.20 | Ppp2r2b | AY251277 | Protein phosphatase 2 (formerly 2A), regulatory subunit B |
| | | | (PR 52), beta isoform |
| 0.20 | | — | — |
| 0.17 | Tram1l1_predicted | — | — |
| 0.17 | Slc6a2 | AB021971 | Solute carrier family 6 (neurotransmitter transporter, |
| | | | noradrenalin), member 2 |
| 0.17 | PAIHC3 | X83231 | Inter-alpha trypsin inhibitor, heavy chain 3 |
| 0.15 | Ace2 | AY881244 | Angiotensin I converting enzyme (peptidyl-dipeptidase A) 2 |
| 0.14 | Calcb | M11596, | CALCITONIN GENE-RELATED PEPTIDE II |
| | | M11596 | PRECURSOR (CGRP-II) (BETA-TYPE CGRP). |
| | | | [Source: SWISSPROT; Acc: P10093] |
| 0.14 | Rbp4 | — | PLASMA RETINOL-BINDING PROTEIN PRECURSOR |
| | | | (PRBP) (RBP). [Source: SWISSPROT; Acc: P04916] |
| 0.09 | | — | — |

TABLE 7

| Nkx6.1 | Pdx1 | Common | Description |
|---|---|---|---|
| 4.20 | 0.86 | Vgf | VGF nerve growth factor inducible |
| 4.07 | 1.08 | Icam1 | Intercellular adhesion molecule 1 |
| 3.84 | 0.95 | | LY6-B ANTIGEN GENE. [Source: RefSeq; Acc: NM_139257] |
| 3.33 | 1.17 | Grin2c | Glutamate receptor, ionotropic, NMDA2C |
| 2.99 | 1.24 | Psmb9 | PROTEASOME SUBUNIT BETA TYPE 9 PRECURSOR (EC 3.4.25.1) |
| 2.80 | 1.22 | Bzrp | PERIPHERAL-TYPE BENZODIAZEPINE RECEPTOR (PBR) (PKBS) |
| 2.79 | 1.23 | Nol3 | Nucleolar protein 3 (apoptosis repressor with CARD domain) |
| 2.31 | 1.13 | Gapd | Similar to glyceraldehyde-3-phosphate dehydrogenase |
| 2.27 | 1.07 | Cxcl10 | Chemokine (C—X—C motif) ligand 10 |
| 2.26 | 0.80 | Prkwnk4 | PROTEIN KINASE, LYSINE DEFICIENT 4; WNK4 SER/THR KINASE. |
| 2.23 | 0.88 | Cyln2 | Cytoplasmic linker 2 |
| 2.19 | 0.99 | Dusp1 | DUAL SPECIFICITY PROTEIN PHOSPHATASE 1 |
| 2.18 | 0.94 | Cstb | CYSTATIN B (LIVER THIOL PROTEINASE INHIBITOR) |
| 2.16 | 1.12 | Pspla1 | Phosphatidylserine-specific phospholipase A1 |
| 2.14 | 1.07 | Gmfg | GLIA MATURATION FACTOR BETA (GMF-BETA). |
| 2.13 | 1.25 | Slc25a10 | Solute carrier family 25 (mitochondrial carrier; dicarboxylate transporter), |
| 2.07 | 0.81 | Cxcl12 | Chemokine (C—X—C motif) ligand 12 |
| 2.07 | 1.21 | Myadm | Myeloid-associated differentiation marker |
| 2.06 | 1.23 | Mx2 | INTERFERON-INDUCED GTP-BINDING PROTEIN MX2. |
| 2.04 | 1.08 | Rab13 | RAS-RELATED PROTEIN RAB-13 (FRAGMENT). |
| 2.02 | 1.22 | Adora1 | Adenosine A1 receptor |
| 2.00 | 1.02 | Ly6c | Ly6-C antigen |
| 0.49 | 1.10 | Snrpn | Small nuclear ribonucleoprotein N |
| 0.48 | 0.95 | Pklr | Pyruvate kinase, liver and RBC |
| 0.42 | 1.05 | Idh1 | Isocitrate dehydrogenase 1 (NADP+), soluble |
| 0.40 | 0.96 | Rbbp7 | Retinoblastoma binding protein 7 |
| 0.37 | 0.77 | Kcnn2 | Potassium intermediate/small conductance calcium-activated channel |
| 0.36 | 0.77 | Gnat3 | G-protein alpha subunit |
| 0.28 | 0.80 | Sstr2 | Somatostatin receptor 2 |
| 0.26 | 1.19 | Lgals2 | GALECTIN-2. [Source: SWISSPROT; Acc: Q9Z144] |
| 0.20 | 0.87 | Ptger2 | Prostaglandin E receptor 2, subtype EP2 |
| 0.20 | 1.03 | Ppp2r2b | Protein phosphatase 2 (formerly 2A), regulatory subunit B (PR 52) |
| 0.14 | 1.06 | Rbp4 | PLASMA RETINOL-BINDING PROTEIN PRECURSOR (PRBP) (RBP). |

1. A method of stimulating growth of a pancreatic islet beta cell, comprising delivering to the cell an exogenous nucleotide sequence encoding a Nkx6.1 protein or an active fragment thereof.

2. The method of claim 1, wherein the cell is in vitro or ex vivo.

3. The method of claim 1, wherein the cell is in vivo.

4. The method of claim 1, wherein the cell is a human pancreatic islet beta cell.

5. The method of claim 1, wherein the exogenous nucleotide sequence is present in a viral vector.

6. The method of claim 1, wherein the exogenous nucleotide sequence is selected from the group consisting of:
the nucleotide sequence of SEQ ID NO: 1;
b) a nucleotide sequence encoding a polypeptide comprising the amino acid sequence of SEQ ID NO:2;
c) a nucleotide sequence that hybridizes under stringent conditions to the nucleotide sequence of (a) or (b) above and has Nkx6.1 activity;
d) a nucleotide sequence having at least 80% homology with a nucleotide sequence of (a), (b) or (c) above and has Nkx6.1 activity; and
e) a nucleotide sequence that differs from (a), (b), (c) or (d) above due to the degeneracy of the genetic code and encodes a polypeptide that has Nkx6.1 activity.

7. The method of claim 1, wherein glucose stimulated insulin secretion is enhanced relative to the amount of glucose stimulated insulin secretion in a cell lacking the exogenous nucleotide sequence.

8-10. (canceled)

11. A method of treating diabetes in a subject in need thereof, comprising delivering to the subject an exogenous nucleotide sequence encoding Nkx6.1 or an active fragment thereof.

12. (canceled)

13. A method of increasing glucose stimulated insulin secretion from pancreatic islet beta cells in a subject, comprising delivering an exogenous nucleotide sequence encoding Nkx6.1 or an active fragment thereof to pancreatic islet beta-cells of the subject.

14-25. (canceled)
